(19) United States
(12) Patent Application Publication
(10) Pub. No.: US 2013/0090364 A1
(43) Pub. Date: Apr. 11, 2013

(54) ANALOGUES FOR THE TREATMENT OR PREVENTION OF FLAVIVIRUS INFECTIONS

(71) Applicant: Vertex Pharmaceuticals Incorporated, Cambridge, MA (US)

(72) Inventors: Laval Chan Chun Kong, Kirkland (CA); Simon Giroux, Cambridge, MA (US); Jagadeeswar T. Reddy, Pierrefonds (CA); Youssef L. Bennani, Lorraine (CA); Sanjoy Kumar Das, Pierrefonds (CA); John Maxwell, Hingham, MA (US); Constantin Yannopoulos, Notre-Dame de I'lle Perrot (CA); Bingcan Liu, Montreal (CA); Jingwang Xu, Framingham, MA (US); Kevin M. Cottrell, Cambridge, MA (US); Mark A. Morris, Somerville, MA (US)

(73) Assignee: Vertex Pharmaceuticals Incorporated, Cambridge, MA (US)

(21) Appl. No.: 13/625,160

(22) Filed: Sep. 24, 2012

Related U.S. Application Data
(63) Continuation of application No. PCT/US2011/029848, filed on Mar. 24, 2011.
(60) Provisional application No. 61/360,315, filed on Jun. 30, 2010.
(60) Provisional application No. 61/316,997, filed on Mar. 24, 2010.

(51) Int. Cl. C07D 495/04

(52) U.S. Cl. CPC C07D 495/04
USPC 514/393; 548/303.7

(57) ABSTRACT

Compounds represented by formula I

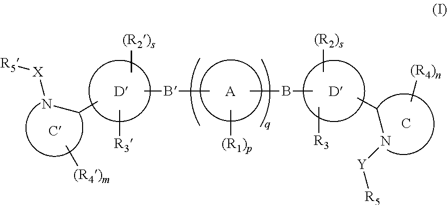

or pharmaceutically acceptable salts thereof, wherein A, B, B′, X, Y, R₁, R₂, R₂′, R₃, R₃′, R₄, R₄′, R₅, R₅′m, n, or p are as defined herein, are useful for treating flaviviridae viral infections.

### CROSS REFERENCE TO RELATED APPLICATIONS

The present application claims the benefit of PCT application number PCT/US2011/029848, filed Mar. 24, 2011, which claims priority to U.S. Provisional Application No. 61/316,997, filed Mar. 24, 2010 and U.S. Provisional Application No. 61/360,315, filed Jun. 30, 2010, which are hereby incorporated by reference in their entirety.

The present invention relates to novel compounds and a method for the treatment or prevention of Flavivirus infections using novel compounds.

Hepatitis is a disease occurring throughout the world. It is generally of viral nature, although there are other causes known. Viral hepatitis is by far the most common form of hepatitis. Nearly 750,000 Americans are affected by hepatitis each year, and out of those, more than 150,000 are infected with the hepatitis C virus (“HCV”).

HCV is a positive-stranded RNA virus belonging to the Flaviviridae family and has close relationship to the pestiviruses that include hog cholera virus and bovine viral diarrhea virus (BVDV). HCV is believed to replicate through the production of a complementary negative-strand RNA template. Due to the lack of efficient culture replication system for the virus, HCV particles were isolated from pooled human plasma and shown, by electron microscopy, to have a diameter of about 50-60 nm. The HCV genome is a single-stranded, positive-sense RNA of about 9,600 bp coding for a polyprotein of 3009-3030 amino-acids, which is cleaved co- and post-translationally into mature viral proteins (core, E1, E2, p7, NS2, NS3, NS4A, NS4B, NS5A, NS5B). It is believed that the structural glycoproteins, E1 and E2, are embedded into a viral lipid envelope and form stable heterodimers. It is also believed that the structural core protein interacts with the viral RNA genome to form the nucleocapsid. The nonstructural proteins designated NS2 to NS5 include proteins with enzymatic functions involved in virus replication and protein processing including a polymerase, protease and helicase.

The main source of contamination with HCV is blood. The magnitude of the HCV infection as a health problem is illustrated by the prevalence among high-risk groups. For example, 60% to 90% of hemophiliacs and more than 80% of intravenous drug abusers in western countries are chronically infected with HCV. For intravenous drug abusers, the prevalence varies from about 28% to 70% depending on the population studied. The proportion of new HCV infections associated with post-transfusion has been markedly reduced lately due to advances in diagnostic tools used to screen blood donors.

Combination of pegylated interferon plus ribavirin is the treatment of choice for chronic HCV infection. This treatment does not provide sustained viral response (SVR) in a majority of patients infected with the most prevalent genotype (1a and 1b). Furthermore, significant side effects prevent compliance to the current regimen and may require dose reduction or discontinuation in some patients.

There is therefore a great need for the development of anti-viral agents for use in treating or preventing Flavivirus infections.

In one aspect, the present invention provides a compound of formula (I):

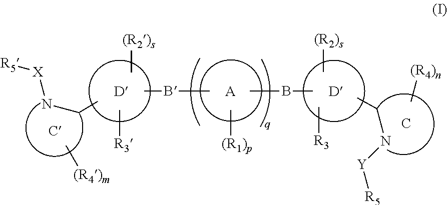

or a pharmaceutically acceptable salt thereof, wherein
each A is independently C₆₋₁₄ aryl, 4-12 membered heterocycle, C₃₋₁₀ cycloalkyl, or 5-12 membered heteroaryl;
B and B′ are each independently absent, C₁₋₆ alkyl, C₂₋₆ alkenyl, or C₂₋₆ alkynyl;
C and C′ are each independently a 4-7 membered heterocycle;
D is a 5,5 membered heterocyclic ring comprising at least one nitrogen atom in the five membered ring adjacent to ring C;
D′ is a 5, 5,6, or a 5,5 membered heterocyclic ring comprising at least one nitrogen atom in the five membered ring adjacent to ring C;
R₁ is halogen, —ORₐ, —NRₐR_{b}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, —P(═O)ORₐOR_{b}, C₁₋₆ alkyl which is unsubstituted or substituted one or more times by R¹⁰, C₂₋₆ alkenyl which is unsubstituted or substituted one or more times by R¹⁰, C₂₋₆ alkynyl which is unsubstituted or substituted one or more times by R¹⁰, or any two occurrences of R₁ can be taken together with the atoms to which they are attached to form a 5-7 cycloalkyl which is unsubstituted or substituted one or more times by R¹¹ or a 5-7 membered heterocycle which is unsubstituted or substituted one or more times by R¹²;
Rₐ—R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl;
Each R₂ and R_{2′}, is independently halogen, C₁₋₁₀ alkyl, C₁₋₆ halogenated alkyl, —(CH₂)₁₋₆OH, —ORₐ, —C(═O)ORₐ, —NRₐR_{b}, —NR_{b}C(═O)Rₐ, —C(O)NRₐR_{b}, —S(O)₀₋₃Rₐ, C₆₋₁₂ aryl, 5-12 membered heterocycle, or 5-12 membered heteroaryl;
R₃ and R₃′ are each independently H, C₁₋₆ alkyl, —(CH₂)₁₋₆OH, C₂₋₆ alkenyl, or C₂₋₆ alkynyl;
R₄ and R₄′ are each independently halogen, —NRₐR_{b}, —C(O)NRₐR_{b}, —(CH₂)₁₋₆OH, C₁₋₆ alkyl, C₁₋₆ halogenated alkyl, hydroxyl, C₆₋₁₄ aryl, or C₁₋₆ alkoxy; wherein two occurrence of R₄ can be taken together with the atoms to which they are attached to form a C₁₋₆ alkenyl which is unsubstituted or substituted one or more times by R¹⁰, a 3-7 cycloalkyl which is unsubstituted or substituted one or more times by R¹¹ or a 4-7 membered heterocycle which is unsubstituted or substituted one or more times by R¹²; wherein two occurrence of R₄′ can be taken together with the atoms to which they are attached to form a C₁₋₆ alkenyl which is unsubstituted or substituted one or more times by R¹⁰, a 3-7 cycloalkyl which is unsubstituted or substituted one or more times by R¹¹ or a 4-7 membered heterocycle which is unsubstituted or substituted one or more times by R¹²;
X and Y are each independently

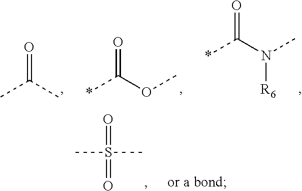

wherein the asterisk (*) indicates the point of attachment to the nitrogen of ring C or C′;
R₅ and R₅′ are each independently H, C₁₋₁₈ alkyl which is unsubstituted or substituted one or more times by R¹⁰, C₂₋₁₂ alkenyl which is unsubstituted or substituted one or more times by R¹⁰, C₂₋₁₂ alkynyl which is unsubstituted or substituted one or more times by R¹⁰, C₆₋₁₄ aryl which is unsubstituted or substituted one or more times by R¹¹, C₇₋₁₆ aralkyl which is unsubstituted or substituted one or more times by R¹¹, 5-12 membered heteroaryl which is unsubstituted or substituted one or more times by R¹¹, 6-18 membered heteroaralkyl which is unsubstituted or substituted one or more times by R¹¹, 3-12 membered heterocycle which is unsubstituted or substituted one or more times by R¹², or 4-18 membered heterocycle-alkyl which is unsubstituted or substituted one or more times by R¹²;
R₆ is H, C₁₋₆ alkyl, or halogenated C₁₋₆ alkyl;
m, and n, are each independently 0, 1, 2, 3 or 4;
p is 0, 1, 2, 3 or 4;
q is 0, 1 or 2;
s is 0, 1, 2, 3 or 4;
R¹⁰ is halogen, —ORₐ, oxo, —NRₐR_{b}, ═NO—R_{c}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b};
R¹¹ is halogen, —ORₐ, —NRₐR_{b}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl; and
R¹² is halogen, —ORₐ, oxo, —NRₐR_{b}, ═NO—R_{c}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

In another aspect, there is provided a method for treating or preventing a Flaviviridae viral infection in a patient comprising administering to the patient a therapeutically effective amount of a compound, composition or combination of the invention.

In another aspect, there is provided a pharmaceutical composition comprising at least one compound of the invention and at least one pharmaceutically acceptable carrier or excipient.

In another aspect, there is provided a combination comprising a compound of the invention and one or more additional agents chosen from viral serine protease inhibitors, viral polymerase inhibitors, viral helicase inhibitors, immunomudulating agents, antioxidant agents, antibacterial agents, therapeutic vaccines, hepatoprotectant agents, antisense agent, inhibitors of HCV NS2/3 protease and inhibitors of internal ribosome entry site (IRES).

In a further aspect, there is provided the use of a compound, composition or combination of the invention for treating or preventing a Flaviviridae viral infection in a human.

In still another aspect, there is provided the use of a compound, composition or combination of the invention for the manufacture of a medicament for treating or preventing a viral Flaviviridae infection in a human.

In one embodiment, compounds of the present invention comprise those wherein the following embodiments are present, either independently or in combination.

In accordance with a further embodiment, the compounds of the present invention are represented by formula (IA):

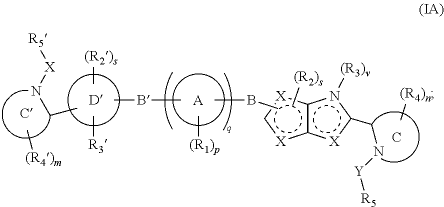

or a pharmaceutically acceptable salt thereof; wherein:

D′ is selected from the group consisting of:

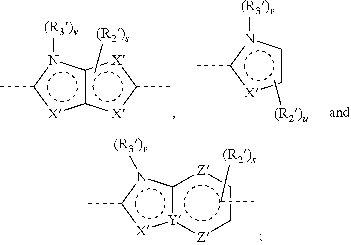

each X and X′ is independently —N—, —S—, or —CH—;
each Z′ is independently —N— or —CH—;
u is 0 or 1; and
each v is independently 0 or 1.

The remainder of the variables for the compounds of formula (IA) are as defined herein for the compounds of formula (I).

In accordance with a further embodiment, the compounds of the present invention are represented by formula (II), (IIIA), or (IIIB):

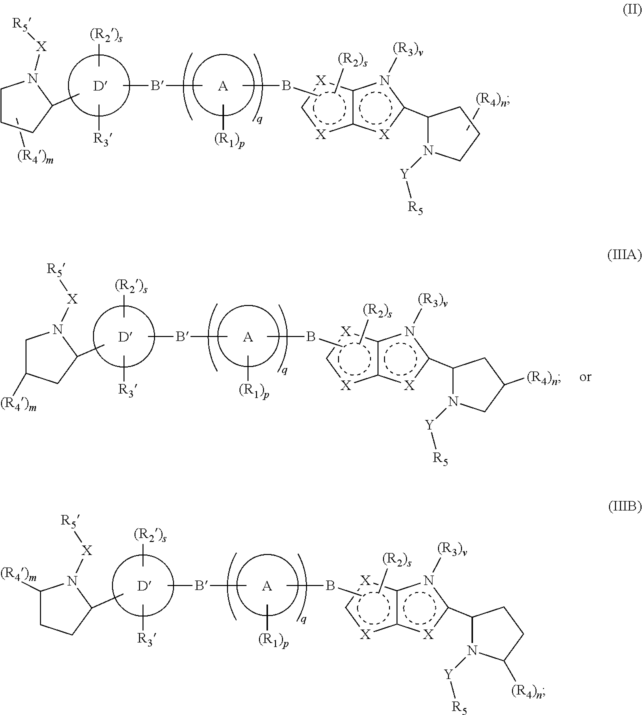

or a pharmaceutically acceptable salt thereof, wherein the variables for the compounds of formula (II), (IIIA), or (IIIB) are as defined herein for the compounds of formulae (I) and (IA).

In accordance with a further embodiment, the compounds of the present invention are represented by formula (IIIC).

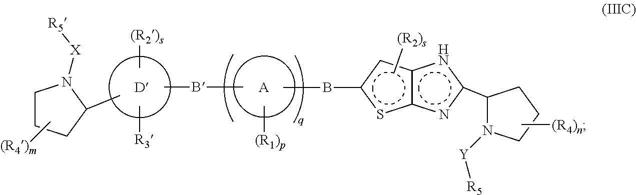

or a pharmaceutically acceptable salt thereof, wherein the variables for the compounds of formula (IIIC) are as defined herein for the compounds of formula (I), (IA), (II), (IIIA), or (IIIB).

In accordance with a further embodiment, the compounds of the present invention are represented by formula (IV) or (V):

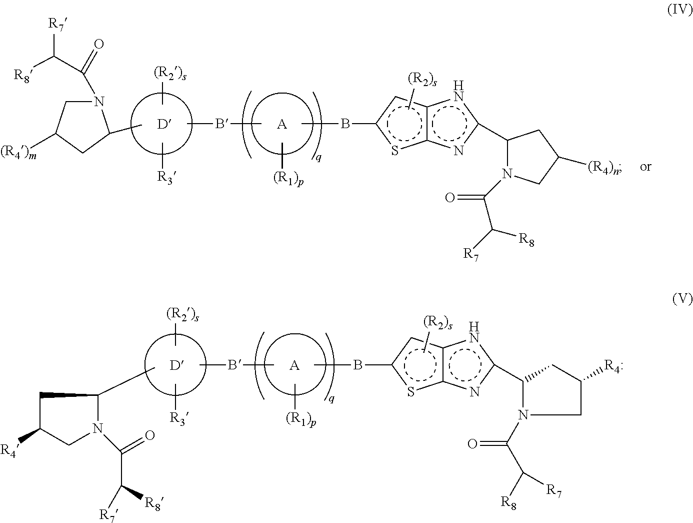

or a pharmaceutically acceptable salt thereof, wherein

R₇ and R₇′ are each independently C₁₋₈ alkyl which is unsubstituted or substituted one or more times by R¹⁰, C₂₋₈ alkenyl which is unsubstituted or substituted one or more times by R¹⁰, C₂₋₈ alkynyl which is unsubstituted or substituted one or more times by R¹⁰, phenyl which is unsubstituted or substituted one or more times by R¹¹, benzyl which is unsubstituted or substituted one or more times by R¹¹, 5-6 membered heteroaryl which is unsubstituted or substituted one or more times by R¹¹, 6-7 membered heteroaralkyl which is unsubstituted or substituted one or more times by R¹¹, 3-6 membered heterocycle which is unsubstituted or substituted one or more times by R¹², or 4-7 membered heterocycle-alkyl which is unsubstituted or substituted one or more times by R¹²;

R₈ and R₈′ are each independently —NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —NR_{b}SO₂Rₐ, or —NR_{b}SO₂NRₐR_{b}, wherein Rₐ—R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl; and

m and n combined are 0, 1, 2, 3 or 4; and

wherein the remainder of the variables for the compounds of formula (IV) or (V) are as defined herein for the compounds of formula (I), (IA), (II), (IIIA), (IIIB), or (IIIC).

In accordance with a further embodiment, the compounds of the present invention are represented by formula (VI) or (VII):

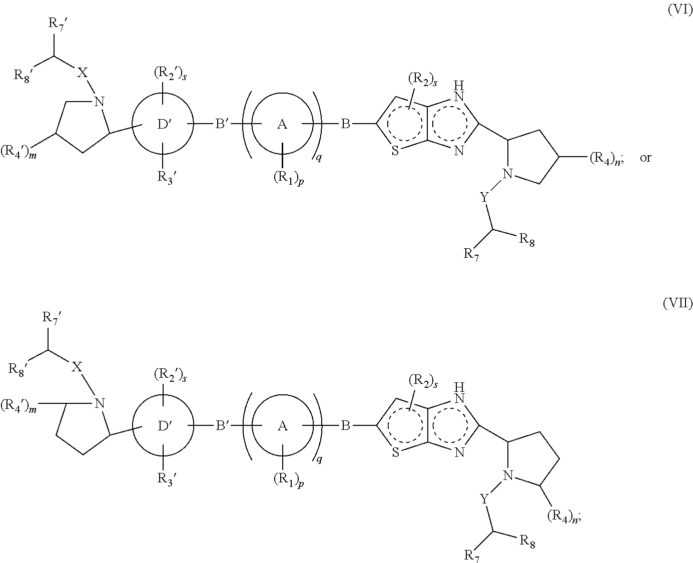

or a pharmaceutically acceptable salt thereof, wherein the variables for the compounds of formula (VI) or (VII) are as defined herein for the compounds of formula (I), (IA), (II), (IIIA), (IIIB), (IIIC), (IV), or (V).

Further embodiments of compounds of formula (I), (IA), (II), (IIIA), (IIIB), (IIIC), (IV), (V), (VI) or (VII) are described below:

According to a further embodiment, A is phenyl, thiophene, thieno[3,2-b]thiophene, pyridine, pyrimidine, naphthyl, benzo[1,3]dioxole, benzooxazole, or triazole

According to a further embodiment, A is phenyl, thiophene, thieno[3,2-b]thiophene, naphtyl, benzo[1,3]dioxole, or benzooxazole.

According to a further embodiment, A is phenyl, thiophene, pyridine, pyrimidine, or triazole.

According to a further embodiment, A is phenyl or thieno[3,2-b]thiophene.

According to a further embodiment, A is phenyl or thiophene.

According to a further embodiment, A is

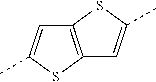

According to a further embodiment, A is

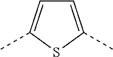

According to a further embodiment, A is

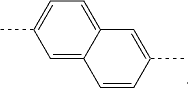

According to a further embodiment, A is

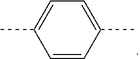

According to a further embodiment, A is a bond.

According to a further embodiment, B and B′ are each independently C₂₋₆ alkynyl or C₁₋₆ alkyl.

According to a further embodiment, B and B′ are each independently —(C═C)— or —(CH₂)₂—.

According to a further embodiment, B and B′ are each —(CH₂)₂—.

According to a further embodiment, B and B′ are each —(C═C)—.

According to a further embodiment, m or n is 2.

According to a further embodiment, m or n is 1.

According to a further embodiment, p is 2.

According to a further embodiment, p is 1.

According to a further embodiment, X and Y are each

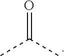

According to a further embodiment, X and Y are each

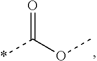

wherein the bond marked with an asterisk (*) indicates the attachment to the nitrogen of ring C or C′.

According to a further embodiment, R₄ and R₄′ are each independently H, halogen, C₁₋₆ alkyl, hydroxyl, phenyl, or C₁₋₄ alkoxy.

According to a further embodiment, R₄ and R₄′ are each independently H, halogen, methyl, ethyl, t-butoxy-, or hydroxyl.

According to a further embodiment, R₄ and R₄′ are each H.

According to a further embodiment, R₄ and R₄′ are each fluoro.

According to a further embodiment, R₄ and R₄′ are each methyl.

According to a further embodiment, R₃ and R₃′ are each H.

According to a further embodiment, R₁ is H, halogen, —ORₐ, —NRₐR_{b}, —C(═O)ORₐ, —C(O)NRₐR_{b}, —C(═O)OH, —NR_{b}C(═O)Rₐ, -hydroxyl, nitro, cyano, —S(O)₀₋₃Rₐ, C₁₋₆ alkyl, C₂₋₆ alkenyl, C₂₋₆ alkynyl, or C₁₋₆ halogenated alkyl.

According to a further embodiment, R₁ is halogen, C₁₋₃ alkyl, hydroxyl, cyano, or C₁₋₃ alkoxy.

According to a further embodiment, R₁ is chloro, fluoro, methyl, hydroxyl, cyano, or methoxy.

According to a further embodiment, R₁ is methyl.

According to a further embodiment, R₁ is H.

According to a further embodiment, R₂ and R₂′ are each independently H, halogen, C₁₋₆ alkyl, —(CH₂)₁₋₃OH, —ORₐ, —C(═O)ORₐ, —C(O)NRₐR_{b}, —C(═O)OH, C₆₋₁₂ aryl, or 5-12 membered heteroaryl, wherein Rₐ—R_{d} are each independently H, C₁₋₁₂ alkyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R₂ and R₂′ are each independently H, halogen, C₁₋₆ alkyl, —(CH₂)₁₋₃OH, —ORₐ, —C(═O)ORₐ, —C(O)NRₐR_{b}, —C(═O)OH, phenyl, or 5-6 membered heteroaryl, wherein Rₐ—R_{d} are each independently H, C₁₋₁₂ alkyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R₂ and R₂′ are each methyl.

According to a further embodiment, R₂ and R₂′ are each iodo.

According to a further embodiment, R₂ and R₂′ are each H.

According to a further embodiment, R₆ is H or C₁₋₃ alkyl.

According to a further embodiment, R₅ and R₅′ are each independently C₁₋₈ alkyl which is unsubstituted or substituted one or more times by R¹⁰, C₂₋₈ alkenyl which is unsubstituted or substituted one or more times by R¹⁰, C₂₋₈ alkynyl which is unsubstituted or substituted one or more times by R¹⁰, phenyl which is unsubstituted or substituted one or more times by R¹¹, C₇₋₈ aralkyl which is unsubstituted or substituted one or more times by R¹¹, 5-6 membered heteroaryl which is unsubstituted or substituted one or more times by R¹¹, 6-8 membered heteroaralkyl which is unsubstituted or substituted one or more times by R¹¹, 3-6 membered heterocycle which is unsubstituted or substituted one or more times by R¹², or 4-8 membered heterocycle-alkyl which is unsubstituted or substituted one or more times by R¹².

According to a further embodiment, R₅ and R₅′ are each independently C₁₋₆ alkyl which is unsubstituted or substituted one or more times by R¹⁰, C₂₋₆ alkenyl which is unsubstituted or substituted one or more times by R¹⁰, C₂₋₆ alkynyl which is unsubstituted or substituted one or more times by R¹⁰, phenyl which is unsubstituted or substituted one or more times by R¹¹, benzyl which is unsubstituted or substituted one or more times by R¹¹, 5-6 membered heteroaryl which is unsubstituted or substituted one or more times by R¹¹, 6-7 membered heteroaralkyl which is unsubstituted or substituted one or more times by R¹¹, 5-6 membered heterocycle which is unsubstituted or substituted one or more times by R¹², or 6-7 membered heterocycle-alkyl which is unsubstituted or substituted one or more times by R¹².

According to a further embodiment, R₅ and R₅′ are each independently C₁₋₆ alkyl which is unsubstituted or substituted one or more times by R¹⁰, C₂₋₆ alkenyl which is unsubstituted or substituted one or more times by R¹⁰, or C₂₋₆ alkynyl which is unsubstituted or substituted one or more times by R¹⁰.

According to a further embodiment, R₅ and R₅′ are each independently C₁₋₁₂ alkyl which is unsubstituted or substituted one or more times by R¹⁰.

According to a further embodiment, R₅ and R₅′ are each independently methyl, ethyl, propyl, isopropyl, butyl, sec-butyl, tert-butyl, pentyl, 2-methylbutane, 3-methylbutane, cyclopropyl, cyclobutyl, cyclopentyl, cyclohexyl, or cyclohexyl(CH₂)—, which in each case is unsubstituted or substituted one or more times by R¹⁰.

According to a further embodiment, R₅ and R₅′ are each independently methyl, ethyl, propyl, isopropyl, butyl, sec-butyl, tert-butyl, pentyl, 2-methylbutane, 3-methylbutane, cyclopropyl, cyclobutyl, cyclopentyl, cyclohexyl, or cyclohexyl(CH₂)—.

According to a further embodiment, R₅ and R₅′ are each independently isopropyl which is unsubstituted or substituted one or more times by R¹⁰.

According to a further embodiment, R₅ and R₅′ are each independently isopropyl which is unsubstituted or substituted one or more times by —OCH₃.

According to a further embodiment, R₅ and R₅′ are each isopropyl.

According to a further embodiment, R₅ and R₅′ are each H or tert-butyl.

According to a further embodiment, R₅ and R₅′ are each independently phenyl which is unsubstituted or substituted one or more times by R¹¹.

According to a further embodiment, R₅ and R₅′ are each independently benzyl which is unsubstituted or substituted one or more times by R¹¹.

According to a further embodiment, R¹⁰ is halogen, —ORₐ, oxo, —NRₐR_{b}, ═NO—R_{c}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, or —NR_{b}SO₂NRₐR_{b}, wherein Rₐ—R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R¹⁰ is —NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —NR_{b}SO₂Rₐ, or —NR_{b}SO₂NRₐR_{b}, wherein Rₐ—R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R¹⁰ is —NRₐR_{b}, —NR_{d}C(═O)NRₐR_{b}, —NR_{b}C(═O)Rₐ, —NR_{b}C(═O)ORₐ, or —NR_{b}SO₂Rₐ, wherein Rₐ, R_{b}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R¹⁰ is —NRₐR_{b} or —NR_{d}C(═O)NRₐR_{b}, wherein Rₐ and R_{b} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R¹⁰ is —NR_{d}C(═O)NRₐR_{b}, wherein Rₐ, R_{b}, are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R¹⁰ is halogen, —ORₐ, oxo, —C(═O)ORₐ, —C(O)NRₐR_{b}, —C(═O)OH, —C(═O)Rₐ, —OC(═O)NRₐR_{b}, —OC(═O)Rₐ, —OC(═O)ORₐ, hydroxyl, cyano, wherein Rₐ—R_{b} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R¹⁰ is halogen, —ORₐ, oxo, —C(═O)ORₐ, —C(O)NRₐR_{b}, —C(═O)OH, —OC(═O)NRₐR_{b}, hydroxyl, or cyano, wherein Rₐ—R_{b} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R¹⁰ is halogen, C₁₋₆ alkoxy, hydroxyl, or NH₂.

According to a further embodiment, R¹⁰ is halogen, hydroxyl, or NH₂.

According to a further embodiment, R¹⁰ is halogen.

According to a further embodiment, R¹¹ is halogen, —ORₐ, —NRₐR_{b}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, or —NR_{b}SO₂NRₐR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl, wherein Rₐ—R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R¹¹ is halogen, —ORₐ, —NRₐR_{b}, —C(═O)ORₐ, —C(O)NRₐR_{b}, —C(═O)OH, —C(═O)Rₐ, —NR_{d}C(═O)NRₐR_{b}, —NR_{b}C(═O)Rₐ, —NR_{b}C(═O)ORₐ, —OC(═O)NRₐR_{b}, —OC(═O)Rₐ, —OC(═O)ORₐ, hydroxyl, cyano, —SO₂NRₐR_{b}, —NR_{b}SO₂Rₐ, C₁₋₆ alkyl, C₂₋₆ alkenyl, C₂₋₆ alkynyl, phenyl, C₇₋₈ aralkyl, 5-6 membered heteroaryl, 6-8 membered heteroaralkyl, 5-6 membered heterocycle, or 6-8 membered heterocycle-alkyl, wherein Rₐ, R_{b}, and R_{d} are each independently are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R¹¹ is halogen, —ORₐ, —NRₐR_{b}, —C(O)NRₐR_{b}, —C(═O)OH, —C(═O)Rₐ, —NR_{d}C(═O)NRₐR_{b}, —NR_{b}C(═O)Rₐ, —NR_{b}C(═O)ORₐ, —OC(═O)NRₐR_{b}, hydroxyl, cyano, C₁₋₆ alkyl, C₂₋₆ alkenyl, C₂₋₆ alkynyl, phenyl, C₇₋₈ aralkyl, 5-6 membered heteroaryl, 6-8 membered heteroaralkyl, 5-6 membered heterocycle, or 6-8 membered heterocycle-alkyl, wherein Rₐ, R_{b}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R¹¹ is halogen, —ORₐ, —NRₐR_{b}, hydroxyl, cyano, or C₁₋₆ alkyl, wherein Rₐ—R_{b} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R¹¹ is halogen, hydroxyl, cyano, or NH₂.

According to a further embodiment, R¹¹ is halogen.

According to a further embodiment, R¹² is halogen, —ORₐ, oxo, —NRₐR_{b}, ═NO—R_{c}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl, wherein Rₐ—R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R¹² is halogen, —ORₐ, oxo, —NRₐR_{b}, —C(═O)ORₐ, —C(O)NRₐR_{b}, —C(═O)OH, —C(═O)Rₐ, —NR_{d}C(═O)NRₐR_{b}, —NR_{b}C(═O)Rₐ, —NR_{b}C(═O)ORₐ, —OC(═O)NRₐR_{b}, —OC(═O)Rₐ, —OC(═O)ORₐ, hydroxyl, cyano, —SO₂NRₐR_{b}, —NR_{b}SO₂Rₐ, C₁₋₆ alkyl, C₂₋₆ alkenyl, C₂₋₆ alkynyl, phenyl, C₇₋₈ aralkyl, 5-6 membered heteroaryl, 6-8 membered heteroaralkyl, 5-6 membered heterocycle, or 6-8 membered heterocycle-alkyl, wherein Rₐ, R_{b}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R¹² is halogen, —ORₐ, oxo, —NRₐR_{b}, —C(O)NRₐR_{b}, —C(═O)OH, —C(═O)Rₐ, —NR_{d}C(═O)NRₐR_{b}, —NR_{b}C(═O)Rₐ, —NR_{b}C(═O)ORₐ, —OC(═O)NRₐR_{b}, hydroxyl, cyano, C₁₋₆ alkyl, C₂₋₆ alkenyl, C₂₋₆ alkynyl, phenyl, C₇₋₈ aralkyl, 5-6 membered heteroaryl, 6-8 membered heteroaralkyl, 5-6 membered heterocycle, or 6-8 membered heterocycle-alkyl, wherein Rₐ, R_{b}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R¹² is halogen, —ORₐ, oxo, —NRₐR_{b}, hydroxyl, cyano, or C₁₋₆ alkyl, wherein Rₐ—R_{b} are each independently H, C₁₋₁₂ alkyl, O₂₋₁₂ alkenyl, O₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

According to a further embodiment, R¹² is halogen.

According to a further embodiment, Rₐ—R_{d} are each independently H, C₁₋₆ alkyl, C₂₋₆ alkenyl, C₂₋₆ alkynyl, phenyl, C₇₋₈ aralkyl, 5-6 membered heteroaryl, 6-8 membered heteroaralkyl, 5-6 membered heterocycle, or 6-8 membered heterocycle-alkyl.

According to a further embodiment, Rₐ and R_{c} are each independently H, C₁₋₆ alkyl, C₂₋₆ alkenyl, C₂₋₆ alkynyl, phenyl, C₇₋₈ aralkyl, 5-6 membered heteroaryl, 6-8 membered heteroaralkyl, 5-6 membered heterocycle, or 6-8 membered heterocycle-alkyl, and R_{b}, and R_{d} are each independently H or C₁₋₃ alkyl.

According to a further embodiment, Rₐ and R_{c} are each independently H, C₁₋₆ alkyl, C₂₋₆ alkenyl, C₂₋₆ alkynyl, phenyl, benzyl, 5-6 membered heteroaryl, 6-8 membered heteroaralkyl, 5-6 membered heterocycle, or 6-8 membered heterocycle-alkyl, and R_{b}, and R_{d} are each independently H or C₁₋₃ alkyl.

According to a further embodiment, Rₐ—R_{d} are each independently H or C₁₋₃ alkyl.

According to a further embodiment, R₈ and R₈′ in formula (IV), (V), (VI), or (VI) are each independently —NRₐR_{b}, —NR_{b}C(═O)Rₐ, or —NR_{b}C(═O)ORₐ, wherein Rₐ—R_{b} are each independently H, C₁₋₆ alkyl, phenyl, benzyl, 5-6 membered heteroaryl, 6-8 membered heteroaralkyl, 5-6 membered heterocycle, or 6-8 membered heterocycle-alkyl.

According to a further embodiment, R₈ and R₈′ in formula (IV), (V), (VI), or (VI) are each independently —NRₐR_{b} or —NR_{b}C(═O)ORₐ, wherein Rₐ—R_{b} are each independently H, C₁₋₆ alkyl, phenyl, benzyl, 5-6 membered heteroaryl, 6-8 membered heteroaralkyl, 5-6 membered heterocycle, or 6-8 membered heterocycle-alkyl.

According to a further embodiment, R₈ and R₈′ in formula (IV), (V), (VI), or (VI) are each independently —NR_{b}C(═O)ORₐ, wherein Rₐ—R_{b} are each independently H, C₁₋₆ alkyl, phenyl, benzyl, 5-6 membered heteroaryl, 6-8 membered heteroaralkyl, 5-6 membered heterocycle, or 6-8 membered heterocycle-alkyl.

According to a further embodiment, R₈ and R₈′ in formula (IV), (V), (VI), or (VI) are each independently —NR_{b}C(═O)ORₐ, wherein Rₐ—R_{b} are each independently H, C₁₋₆ alkyl, phenyl, tetrahydrofuran, or benzyl.

According to a further embodiment, R₈ and R₈′ in formula (IV), (V), (VI), or (VI) are each independently —NR_{b}C(═O)ORₐ, wherein Rₐ is C₁₋₆ alkyl and R_{b} is H or methyl.

According to a further embodiment, R₈ and R₈′ in formula (IV), (V), (VI), or (VI) are each independently —NR_{b}C(═O)ORₐ, wherein Rₐ is C₁₋₆ alkyl and R_{b} is H.

According to a further embodiment, R₈ and R₈′ in formula (IV), (V), (VI), or (VI) are each independently —NR_{b}C(═O)ORₐ, wherein Rₐ is methyl and R_{b} is H.

According to a further embodiment, R₇ and R₇′ in formula (IV), (V), (VI), or (VI) are each independently C₁₋₈ alkyl, C₂₋₈ alkenyl, C₂₋₈ alkynyl, phenyl, benzyl, 5-6 membered heteroaryl, 6-7 membered heteroaralkyl, 3-6 membered heterocycle, or 4-7 membered heterocycle-alkyl;

According to a further embodiment, R₇ and R₇′ in formula (IV), (V), (VI), or (VI) are each independently phenyl.

According to a further embodiment, R₇ and R₇′ in formula (IV), (V), (VI), or (VI) are each independently C₁₋₆ alkyl.

According to a further embodiment, R₇ and R₇′ in formula (IV), (V), (VI), or (VI) are each independently methyl, ethyl, propyl, isopropyl, butyl, sec-butyl, tert-butyl, pentyl, 2-methylbutane, 3-methylbutane, cyclopropyl, cyclobutyl, cyclopentyl, or cyclohexyl.

According to a further embodiment, R₇ and R₇′ in formula (IV), (V), (VI), or (VI) are each isopropyl.

According to a further embodiment, as valency allows in B, B′, Rₐ—R_{d}, R₁, R₂, R₂′, R₃, R₃′, R₄, R₄′, R¹⁰, R¹¹ and R¹² each of alkyl, alkenyl, alkynyl, alkoxy, aryl, aralkyl, heteroaryl, heteroaralkyl, heterocycle, or heterocycle-alkyl is independently unsubstituted or substituted one or more times by halogen, —ORₐ, —NR_{a′}R_{b′}, C(═O)OR_{a′}, —C(O)NR_{a′}R_{b′}, —C(═O)OH, hydroxyl, nitro, azido, or cyano, wherein R_{a′}—R_{d′}, are each independently H, C₁₋₁₂ alkyl.

According to a further embodiment, as valency allows in B, B′, Rₐ—R_{d}, R₁, R₂, R₂′, R₃, R₃′, R₄, R₄′, R¹⁰, R¹¹ and R¹² each of alkyl, alkenyl, alkynyl, alkoxy, aryl, aralkyl, heteroaryl, heteroaralkyl, heterocycle, or heterocycle-alkyl is independently unsubstituted or substituted one time by halogen.

According to a further embodiment, as valency allows in B, B′, Rₐ—R_{d}, R₁, R₂, R₂′, R₃, R₃′, R₄, R₄′, R¹⁰, R¹¹ and R¹² each of alkyl, alkenyl, alkynyl, alkoxy, aryl, aralkyl, heteroaryl, heteroaralkyl, heterocycle, or heterocycle-alkyl is independently unsubstituted or substituted one time by fluoro.

In accordance with the present invention, the compounds are selected from compounds as defined in the formulas (I), (IA), (II), (IIIA), (IIIB), (IIIC), (IV), (V), (VI) or (VII) wherein:
A is C₆₋₁₄ aryl, 5-12 membered heteroaryl, or a bond;
B and B′ are each independently —(C═C)— or —(CH₂)₂—;
R₁ is H, halogen, —ORₐ, —NRₐR_{b}, —C(═O)ORₐ, —C(O)NRₐR_{b}, —C(═O)OH, —NR_{b}C(═O)Rₐ, hydroxyl, nitro, cyano, —S(O)₀₋₃Rₐ, —C₁₋₆ alkyl, C₂₋₆ alkenyl, C₂₋₆ alkynyl, or C₁₋₆ halogenated alkyl;
R₂ and R₂′ are each independently H, methyl, or iodo;
m and n are each independently 0, 1 or 2;
p is 0, 1 or 2;
R₃ and R₃′ are H;
R₄ and R₄′ are each independently H, halogen, C₁₋₆ alkyl, hydroxyl, phenyl, or C₁₋₄ alkoxy;
X and Y are

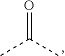

R₅ and R₅′ are each independently C₁₋₁₂ alkyl which is unsubstituted or substituted one or more times by R¹⁰.

In accordance with the present invention, the compounds are selected from compounds as defined in the formulas wherein:
A is C₆₋₁₄ aryl, 5-12 membered heteroaryl, or a bond;
B and B′ are each independently —(C═C)— or —(CH₂)₂—;
R₁ is H or methyl;
R₂ and R₂′ are each independently H, methyl or iodo;
m and n are each independently 0, 1 or 2;
p is 0, 1 or 2;
R₃ and R₃′ are H;
R₄ and R₄′ are each independently H, halogen, C₁₋₆ alkyl, hydroxyl, phenyl, or C₁₋₄ alkoxy;
X and Y are

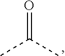

R₅ and R₅′ are each independently C₁₋₁₂ alkyl which is unsubstituted or substituted one or more times by R¹⁰.

In accordance with the present invention, the compounds are selected from compounds as defined in the formulas (I), (IA), (II), (IIIA), (IIIB), (IIIC), (IV), (V), (VI) or (VII) wherein:
A is phenyl, thiophene, thieno[3,2-b]thiophene, pyridine, pyrimidine, naphthyl, benzo[1,3]dioxole, benzooxazole, or triazole;
B and B′ are each independently —(C═C)— or —(CH₂)₂—;
R₁ is H or methyl;
R₂ and R₂′ are each independently H, methyl or iodo;
m and n are each independently 0, 1 or 2;
p is 0, 1 or 2;
R₃ and R₃′ are H;
R₄ and R₄′ are each independently H, halogen, C₁₋₆ alkyl, hydroxyl, phenyl, or C₁₋₄ alkoxy;
X and Y are

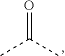

R₅ and R₅′ are each independently C₁₋₁₂ alkyl which is unsubstituted or substituted one or more times by R¹⁰.

In accordance with the present invention, the compounds are selected from compounds as defined in the formulas (I), (IA), (II), (IIIA), (IIIB), (IIIC), (IV), (V), (VI) or (VII) wherein:
A is phenyl, thiophene, thieno[3,2-b]thiophene, naphthyl, benzo[1,3]dioxole, or benzooxazole;
B and B′ are each independently —(C═C)— or —(CH₂)₂—;
R₁ is H, halogen, —ORₐ, —NRₐR_{b}, —C(═O)ORₐ, —C(O)NRₐR_{b}, —C(═O)OH, —NR_{b}C(═O)Rₐ, hydroxyl, nitro, cyano, —S(O)₀₋₃Rₐ, —C₁₋₆ alkyl, C₂₋₆ alkenyl, C₂₋₆ alkynyl, or C₁₋₆ halogenated alkyl;
R₂ and R₂′ are each independently H, methyl or iodo;
m and n are each independently 0, 1 or 2;
p is 0, 1 or 2;
R₃ and R₃′ are H;
R₄ and R₄′ are each independently H, halogen, C₁₋₆ alkyl, hydroxyl, phenyl, or C₁₋₄ alkoxy;
X and Y are each

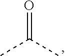

R₅ and R₅′ are each independently C₁₋₁₂ alkyl which is unsubstituted or substituted one or more times by R¹⁰;
R₇ and R₇′ are each independently C₁₋₈ alkyl which is unsubstituted or substituted one or more times by R¹⁰, C₂₋₈ alkenyl which is unsubstituted or substituted one or more times by R¹⁰, C₂₋₈ alkynyl which is unsubstituted or substituted one or more times by R¹⁰, phenyl which is unsubstituted or substituted one or more times by R¹¹, benzyl which is unsubstituted or substituted one or more times by R¹¹, 5-6 membered heteroaryl which is unsubstituted or substituted one or more times by R¹¹, 6-7 membered heteroaralkyl which is unsubstituted or substituted one or more times by R¹¹, 3-6 membered heterocycle which is unsubstituted or substituted one or more times by R¹², or 4-7 membered heterocycle-alkyl which is unsubstituted or substituted one or more times by R¹²; and
R₈ and R₈′ are each independently —NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —NR_{b}SO₂Rₐ, —NR_{b}SO₂NRₐR_{b}, wherein Rₐ—R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

In some embodiments, the compounds of this invention are represented in Tables 1A, 1B, 3, or 4. In certain embodiments, the variables used herein are as defined in the specific embodiments as shown in Tables 1A, 1B, 3, or 4.

In one embodiment in the compounds of the present invention R₁ is halogen, —ORₐ, —NRₐR_{b}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, —P(═O)ORₐOR_{b}, C₁₋₆ alkyl which is unsubstituted or substituted one or more times by R¹⁰, C₂₋₆ alkenyl which is unsubstituted or substituted one or more times by R¹⁰, C₂₋₆ alkynyl which is unsubstituted or substituted one or more times by R¹⁰;

In one embodiment in the compounds of the present invention, herein as valency allows in B, B′, Rₐ—R_{d}, R₁, R₂, R₂′, R₃, R₃′, R₄, R₄′, R₁₀, R₁₁ and R₁₂ each of alkyl, alkenyl, alkynyl, alkoxy, aryl, aralkyl, heteroaryl, heteroaralkyl, heterocycle, or heterocycle-alkyl is independently unsubstituted or substituted one or more times by halogen, —OR_{a′}, oxo, —NR_{a′}R_{b′}, ═NO—R_{c}, —C(═O)OR_{a′}, —C(O)NR_{a′}R_{b′}, —C(═O)OH, —C(═O)R_{a′}, —C(═NOR_{c})R_{a′}, —C(═NR_{c′})NR_{a′}R_{b′}, —NR_{d′}C(═O)NR_{a′}R_{b′}, —NR_{b′}C(═O)R_{a′}, —NR_{d′}C(═NR_{c′})NR_{a′}R_{b′}, —NR_{b′}C(═O)OR_{a′}, —OC(═O)NR_{a′}R_{b′}, —OC(═O)R_{a′}, —OC(═O)OR_{a′}, hydroxyl, nitro, azido, cyano, —S(O)₀₋₃R_{a′}, —SO₂NR_{a′}R_{b′}, —NR_{b′}SO₂R_{a′}; wherein Rₐ—R_{d′} are each independently H, C₁₋₁₂ alkyl.

In one embodiment in the compounds of the present invention, when D′ is a 5 membered ring, R₂ is halogen, C₁₋₁₀ alkyl, C₁₋₆ halogenated alkyl, —(CH₂)₁₋₆OH, —NR_{b}C(═O)Rₐ, C₆₋₁₂ aryl, or 5-12 membered heteroaryl. In particular R₂′ is methyl, trifluoromethyl, iodo, CH₂OH, or NHC(O)CH₃.

In one embodiment in the compounds of the present invention p is 0, 1 or 2.

In one embodiment in the compounds of the present invention p is 0 or 1.

In one embodiment in the compounds of the present invention p is 0.

In one embodiment in the compounds of the present invention p is 2.

In one embodiment in the compounds of the present invention R₄ and R₄′ are H.

In one embodiment in the compounds of the present invention R₁ is halogen, C₁₋₃ alkyl, hydroxyl, cyano, or C₁₋₃ alkoxy.

In one embodiment in the compounds of the present invention R₁ is chloro, fluoro, methyl, hydroxyl, cyano, or methoxy.

In one embodiment in the compounds of the present invention n R₁ is H.

A compound according to claim 31, wherein R¹⁰ is halogen, —ORₐ, oxo, —C(═O)ORₐ, —C(O)NRₐR_{b}, —C(═O)OH, —C(═O)Rₐ, —OC(═O)NRₐR_{b}, —OC(═O)Rₐ, —OC(═O)ORₐ, hydroxyl, cyano, wherein Rₐ—R_{b} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

In one embodiment in the compounds of the present invention R¹¹ is halogen, —ORₐ, —NRₐR_{b}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, or —NR_{b}SO₂NRₐR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl, wherein Rₐ—R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

In one embodiment in the compounds of the present invention R¹¹ is halogen, —ORₐ, —NRₐR_{b}, —C(═O)ORₐ, —C(O)NRₐR_{b}, —C(═O)OH, —C(═O)Rₐ, —NR_{d}C(═O)NRₐR_{b}, —NR_{b}C(═O)Rₐ, —NR_{b}C(═O)ORₐ, —OC(═O)NRₐR_{b}, —OC(═O)Rₐ, —OC(═O)ORₐ, hydroxyl, cyano, —SO₂NRₐR_{b}, —NR_{b}SO₂Rₐ, C₁₋₆ alkyl, C₂₋₆ alkenyl, C₂₋₆ alkynyl, phenyl, C₇₋₈ aralkyl, 5-6 membered heteroaryl, 6-8 membered heteroaralkyl, 5-6 membered heterocycle, or 6-8 membered heterocycle-alkyl, wherein Rₐ, R_{b}, and R_{d} are each independently are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

In one embodiment in the compounds of the present invention R¹¹ is halogen, —ORₐ, —NRₐR_{b}, —C(O)NRₐR_{b}, —C(═O)OH, —C(═O)Rₐ, —NR_{d}C(═O)NRₐR_{b}, —NR_{b}C(═O)Rₐ, —NR_{b}C(═O)ORₐ, —OC(═O)NRₐR_{b}, hydroxyl, cyano, C₁₋₆ alkyl, C₂₋₆ alkenyl, C₂₋₆ alkynyl, phenyl, C₇₋₈ aralkyl, 5-6 membered heteroaryl, 6-8 membered heteroaralkyl, 5-6 membered heterocycle, or 6-8 membered heterocycle-alkyl, wherein Rₐ, R_{b}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

In one embodiment in the compounds of the present invention R¹¹ is halogen, —ORₐ, —NRₐR_{b}, hydroxyl, cyano, C₁₋₆ alkyl, wherein Rₐ—R_{b} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

In one embodiment in the compounds of the present invention R¹² is halogen, —ORₐ, oxo, —NRₐR_{b}, ═NO—R_{c}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl, wherein Rₐ—R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

In one embodiment in the compounds of the present invention R¹² is halogen, —ORₐ, oxo, —NRₐR_{b}, —C(═O)ORₐ, —C(O)NRₐR_{b}, —C(═O)OH, —C(═O)Rₐ, —NR_{d}C(═O)NRₐR_{b}, —NR_{b}C(═O)Rₐ, —NR_{b}C(═O)ORₐ, —OC(═O)NRₐR_{b}, —OC(═O)Rₐ, —OC(═O)ORₐ, hydroxyl, cyano, —SO₂NRₐR_{b}, —NR_{b}SO₂Rₐ, C₁₋₆ alkyl, C₂₋₆ alkenyl, C₂₋₆ alkynyl, phenyl, C₇₋₈ aralkyl, 5-6 membered heteroaryl, 6-8 membered heteroaralkyl, 5-6 membered heterocycle, or 6-8 membered heterocycle-alkyl, wherein Rₐ, R_{b}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

In one embodiment in the compounds of the present invention R¹² is halogen, —ORₐ, oxo, —NRₐR_{b}, —C(O)NRₐR_{b}, —C(═O)OH, —C(═O)Rₐ, —NR_{d}C(═O)NRₐR_{b}, —NR_{b}C(═O)Rₐ, —NR_{b}C(═O)ORₐ, —OC(═O)NRₐR_{b}, hydroxyl, cyano, C₁₋₆ alkyl, C₂₋₆ alkenyl, C₂₋₆ alkynyl, phenyl, C₇₋₈ aralkyl, 5-6 membered heteroaryl, 6-8 membered heteroaralkyl, 5-6 membered heterocycle, or 6-8 membered heterocycle-alkyl, wherein Rₐ, R_{b}, and R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

In one embodiment in the compounds of the present invention R¹² is halogen, —ORₐ, oxo, —NRₐR_{b}, hydroxyl, cyano, C₁₋₆ alkyl, wherein Rₐ—R_{b} are are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

In one embodiment in the compounds of the present invention wherein as valency allows in B, B′, Rₐ—R_{d}, R₁, R₂, R₂′, R₃, R₃′, R₄, R₄′, R¹⁰, R¹¹ and R¹² each of alkyl, alkenyl, alkynyl, alkoxy, aryl, aralkyl, heteroaryl, heteroaralkyl, heterocycle, or heterocycle-alkyl is independently unsubstituted or substituted one or more times by halogen, —ORₐ, —NR_{a′}R_{b′}, C(═O)OR_{a′}, —C(O)NR_{a′}R_{b′}, —C(═O)OH, hydroxyl, nitro, azido, cyano, wherein R_{a′}, —R_{d′} are each independently H, C₁₋₁₂ alkyl.

In one embodiment in the compounds of the present invention wherein as valency allows in B, B′, Rₐ—R_{d}, R₁, R₂, R₂′, R₃, R₃′, R₄, R₄′, R¹⁰, R¹¹ and R¹² each of alkyl, alkenyl, alkynyl, alkoxy, aryl, aralkyl, heteroaryl, heteroaralkyl, heterocycle, or heterocycle-alkyl is independently unsubstituted or substituted one time by halogen.

In one embodiment in the compounds of the present invention wherein as valency allows in B, B′, Rₐ—R_{d}, R₁, R₂, R₂′, R₃, R₃′, R₄, R₄′, R¹⁰, R¹¹ and R¹² each of alkyl, alkenyl, alkynyl, alkoxy, aryl, aralkyl, heteroaryl, heteroaralkyl, heterocycle, or heterocycle-alkyl is independently unsubstituted or substituted one time by fluoro.

In accordance with a first preferred embodiment, the compounds of the present invention are represented by formula (IV):

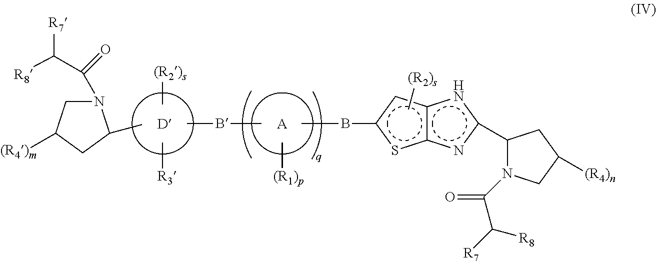

or a pharmaceutically acceptable salt thereof, wherein

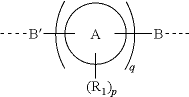

is selected from the group consisting of:

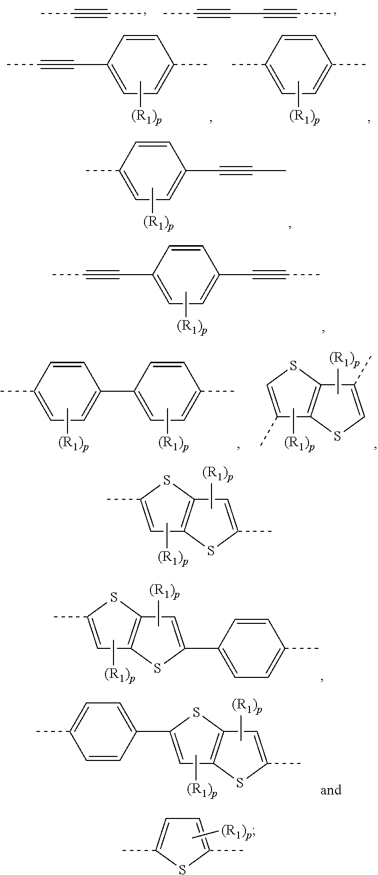

R₇ and R₇′ are each independently C₁₋₈ alkyl which is unsubstituted or substituted one or more times by R¹⁰, C₂₋₈ alkenyl which is unsubstituted or substituted one or more times by R¹⁰, C₂₋₈ alkynyl which is unsubstituted or substituted one or more times by R¹⁰, phenyl which is unsubstituted or substituted one or more times by R¹¹, benzyl which is unsubstituted or substituted one or more times by R¹¹, 5-6 membered heteroaryl which is unsubstituted or substituted one or more times by R¹¹, 6-7 membered heteroaralkyl which is unsubstituted or substituted one or more times by R¹¹, 3-6 membered heterocycle which is unsubstituted or substituted one or more times by R¹², or 4-7 membered heterocycle-alkyl which is unsubstituted or substituted one or more times by R¹²;
R₈ and R₈′ are each independently —NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —NR_{b}SO₂Rₐ, or —NR_{b}SO₂NRₐR_{b}, wherein Rₐ—R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl; and
m and n combined are 0, 1, 2, 3 or 4; and
   wherein the remainder of the variables for the compounds of formula (IV) are as defined herein for the compounds of formula (I), (IA), (II), (IIIA), (IIIB), or (IIIC).

In accordance with a second preferred embodiment, the compounds of formula (IV) are represented by formula (V):

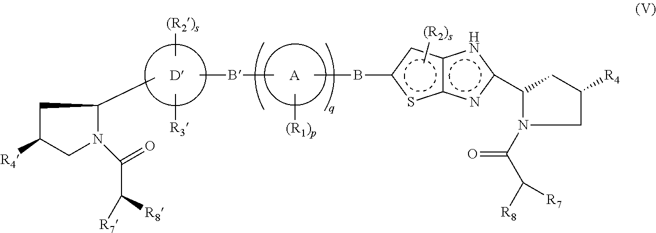

or a pharmaceutically acceptable salt thereof; and wherein the variables for the compounds of formula (V) are as defined herein for the compounds of formula (I), (IA), (II), (IIIA), (IIIB), (IIIC), or (IV).

In accordance with a third preferred embodiment of the compounds of formula (IV) or (V),

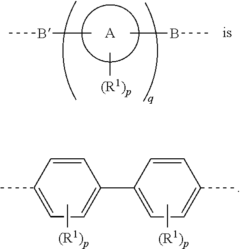

In accordance with a fourth embodiment of the compounds of formula (IV) or (V),

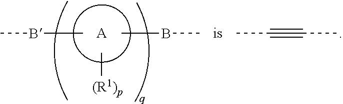

In accordance with a fifth embodiment of the compounds of formula (IV) or (V),

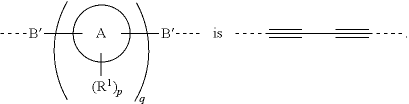

Preferably for the first, second, third, fourth and fifth preferred embodiments, R₄ and R₄′ are methyl. More preferably, R₄ and R_{4′} are methyl and m and n are 1.

In accordance with a sixth preferred embodiment of the compounds of formula (IV) or (V), D′ is:

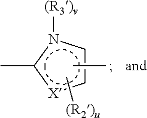

alternatively, the remainer of the variables are as defined in the first through the fifth preferred embodiments.

In accordance with a seventh preferred embodiment of the compounds of formula (IV) or (V), D′ is:

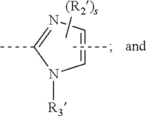

alternatively, the remainer of the variables are as defined in the first through the fifth preferred embodiments.

In accordance with an eighth preferred embodiment, the compounds of formula (IV) or (V) are represented by formula (VI):

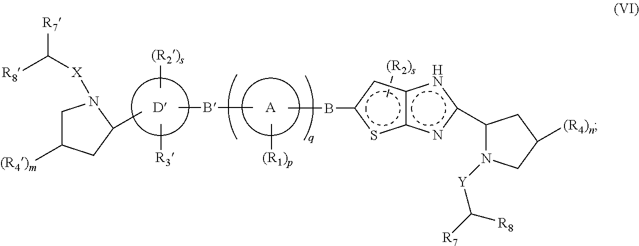

or a pharmaceutically acceptable salt thereof; and wherein R₇ and R₇′ are each independently C₁₋₈ alkyl which is unsubstituted or substituted one or more times by R¹⁰, C₂₋₈ alkenyl which is unsubstituted or substituted one or more times by R¹⁰, C₂₋₈ alkynyl which is unsubstituted or substituted one or more times by R¹⁰, phenyl which is unsubstituted or substituted one or more times by R¹¹, benzyl which is unsubstituted or substituted one or more times by R¹¹, 5-6 membered heteroaryl which is unsubstituted or substituted one or more times by R¹¹, 6-7 membered heteroaralkyl which is unsubstituted or substituted one or more times by R¹¹, 3-6 membered heterocycle which is unsubstituted or substituted one or more times by R¹², or 4-7 membered heterocycle-alkyl which is unsubstituted or substituted one or more times by R¹²;
R₈ and R₈′ are each independently —NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —NR_{b}SO₂Rₐ, or —NR_{b}SO₂NRₐR_{b}, wherein Rₐ—R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl; and
m and n combined are 0, 1, 2, 3 or 4; and wherein the remainder of the variables for the compounds of formula (VI) are as defined herein for the compounds of formula (I), (IA), (II), (IIIA), (IIIB), (IIIC), (IV), or (V).

In accordance with a ninth preferred embodiment, the compounds of formula (IV) or (V) are represented by formula (VII):

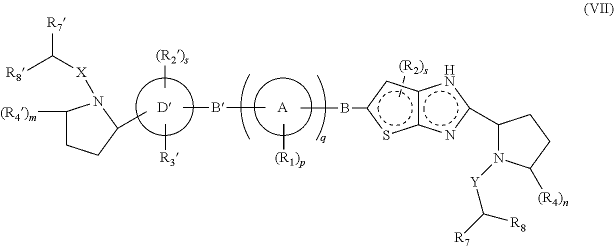

or a pharmaceutically acceptable salt thereof; and wherein the variables for the compounds of formula (VII) are as defined herein for the compounds of formula (I), (IA), (II), (IIIA), (IIIB), (IIIC), (IV), (V), or (VI).

In accordance with a tenth preferred embodiment, for the compound of the invention as described in the first through ninth preferred embodiment, R₄ and R₄′ are methyl. Alternatively, R₄ and R₄′ are methyl and m and n are 1.

In accordance with an eleventh preferred embodiment, for the compound of the invention as described in the first through tenth preferred embodiments,

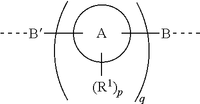

is selected from the group consisting of:

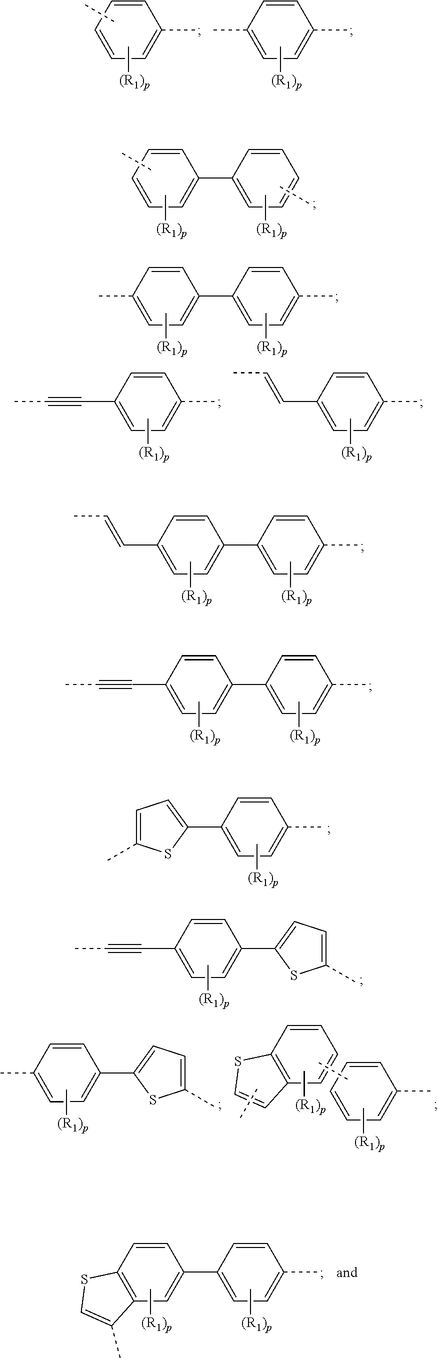

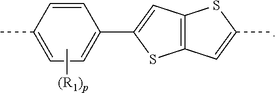

In accordance with a twelfth preferred embodiment, for the compound of the invention as described in the first through eleventh preferred embodiments,

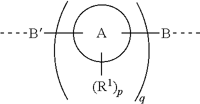

is selected from the group consisting of:

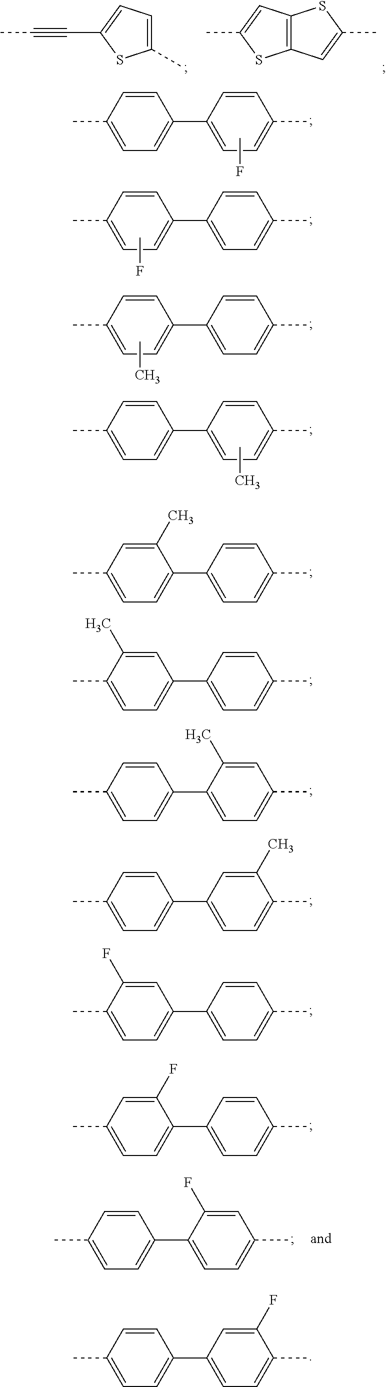

In accordance with a thirteenth preferred embodiment, for the compound of the invention as described in the first through twelfth preferred embodiment D′ is selected from the group consisting of:

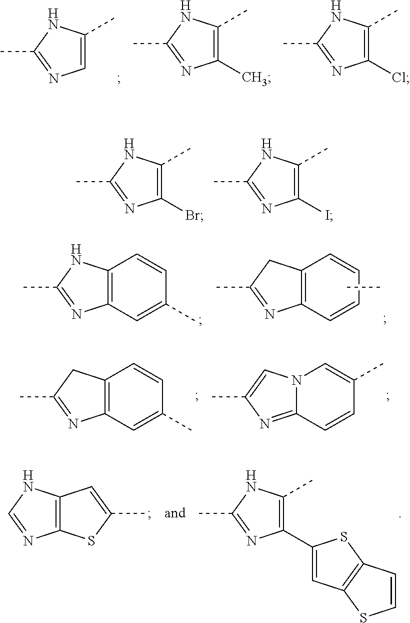

In accordance with a fourteenth preferred embodiment, for the compound of the invention as described in the first through thirteenth preferred embodiments, R₁ is halogen, C₁₋₄ alkyl which is unsubstituted or substituted one or more times by R₁₀, —C(═O)ORₐ, —C(O)NRₐR_{b}, hydroxyl, cyano, or C₁₋₃ alkoxy.

In accordance with a fiftheenth preferred embodiment, for the compound of the invention as described in the first through fourteenth preferred embodiments, R₁ is chloro, fluoro, bromo, methyl, ethyl, propyl, butyl, —CH₂OH, difluoromethyl, trifluoromethyl, —C(═O)ORₐ, hydroxyl, cyano, or methoxy.

In accordance with a sixteenth preferred embodiment, for the compound of the invention as described in the first through fifteenth preferred embodiments, R₂ and R₂′ is fluoro, methyl, trifluoromethyl, iodo, CH₂OH, or NHC(O)CH₃.

In accordance with a seventeenth preferred embodiment, for the compound of the invention as described in the first through sixteenth preferred embodiments, s is 0.

In accordance with a eighteenth preferred embodiment, for the compound of the invention as described in the first through seventeenth preferred embodiments, R₃′ is H or methyl.

In accordance with a nineteenth preferred embodiment, for the compound of the invention as described in the first through eighteenth preferred embodiments, R₄ and R₄′ are each independently halogen, methyl, ethyl, isopropyl, di-fluoromethyl, di-fluoroethyl, trifluoromethyl, tri-fluoroethyl, —CH₂OH, —NRₐN_{b}, t-butoxy-, or hydroxyl; or two R₄ groups together with the atoms to which they are attached form fused cyclopropyl, spiro cyclopropyl or

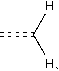

two R₄′ groups together with the atoms to which they are attached form fused cyclopropyl, spiro cyclopropyl or

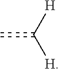

In accordance with a twentieth preferred embodiment, for the compound of the invention as described in the first through nineteenth preferred embodiments, R₈ and R₈′ are each independently —NRₐR_{b}, —NR_{b}C(═O)Rₐ, —NR_{b}C(═O)ORₐ, wherein Rₐ—R_{b} are each independently H, C₁₋₆ alkyl, phenyl, benzyl, 5-6 membered heteroaryl, 6-8 membered heteroaralkyl, 5-6 membered heterocycle, or 6-8 membered heterocycle-alkyl.

In accordance with a twenty-first preferred embodiment, for the compound of the invention as described in the first through twentieth preferred embodiments, R₈ and R₈′ in formulas (IV), are each independently —NR_{b}C(═O)ORₐ, wherein Rₐ—R_{b} are each independently H, C₁₋₆ alkyl, phenyl, tetrahydrofuran, or benzyl.

In accordance with a twenty-second preferred embodiment, for the compound of the invention as described in the first through twentyfirst preferred embodiment, R₇ and R₇′ are each independently phenyl which is unsubstituted or substituted one or more times by R¹¹.

In accordance with a twenty-third preferred embodiment, for the compound of the invention as described in the first through twenty-second preferred embodiments, R₇ and R₇′ are each independently, C₁₋₆ alkyl which is unsubstituted or substituted one or more times by R¹⁰.

In accordance with a twenty-fourth preferred embodiment, for the compound of the invention as described in the first through twenty-third preferred embodiments, R₇ and R₇′ are each independently methyl, ethyl, propyl, isopropyl, methoxyisopropyl, butyl, sec-butyl, tert-butyl, pentyl, 2-methylbutane, 3-methylbutane, cyclopropyl, cyclobutyl, cyclopentyl, or cyclohexyl.

In accordance with a twenty-fifth preferred embodiment, for the compound of the invention as described in the first through twenty-fourth preferred embodiments, R₇ and R₈ or R_{7′} and R_{8′} together with the carbon to which they are attached are each independently:

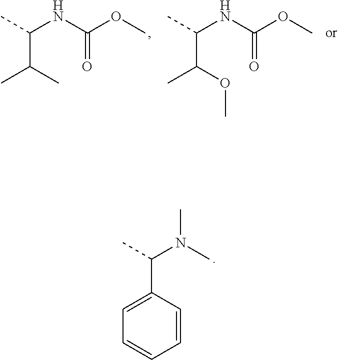

In accordance with a twenty-sixth preferred embodiment, for the compound of the invention as described in the first through twenty-fifth preferred embodiments, R₇ and R₈ or R_{7′} and R_{8′} together with the carbon to which they are attached are each:

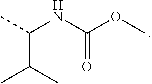

In accordance with a twenty-seventh preferred embodiment, for the compound of the invention as described in the first through twenty-sixth preferred embodiment, R¹⁰ is —NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —NR_{b}SO₂Rₐ, or —NR_{b}SO₂NRₐR_{b}.

In accordance with a twenty-eight preferred embodiment, for the compound of the invention as described in the first through twenty-seventh preferred embodiment, R¹⁰ is —NRₐR_{b}, —NR_{d}C(═O)NRₐR_{b}, —NR_{b}C(═O)Rₐ, —NR_{b}C(═O)ORₐ, or —NR_{b}SO₂Rₐ.

In accordance with a twenty-ninth preferred embodiment, for the compound of the invention as described in the first through twenty-eighth preferred embodiments, Rₐ—R_{d} are each independently H, C₁₋₆ alkyl, C₂₋₆ alkenyl, C₂₋₆ alkynyl, phenyl, C₇₋₈ aralkyl, 5-6 membered heteroaryl, 6-8 membered heteroaralkyl, 5-6 membered heterocycle, or 6-8 membered heterocycle-alkyl.

In accordance with a thirtieth preferred embodiment, for the compound of the invention as described in the first through twenty-ninth preferred embodiment, Rₐ—R_{d} are each independently H or C₁₋₃ alkyl.

Another embodiment of the invention is a compound is of formula (VIII):

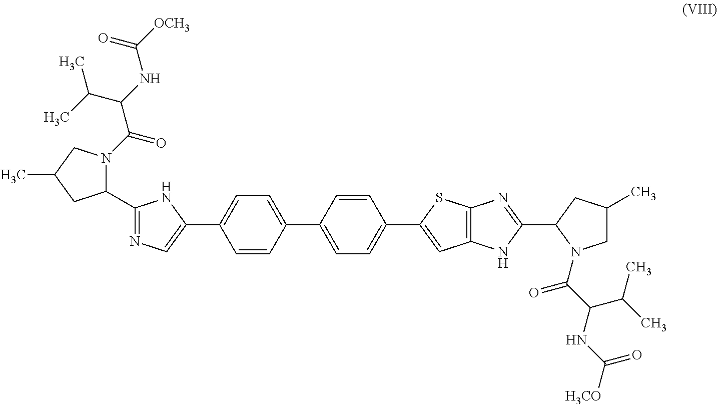 [SMILES: COC(=O)NC(C(=O)N1CC(C)CC1c1ncc(-c2ccc(-c3ccc(-c4cc5[nH]c(C6CC(C)CN6C(=O)C(NC(=O)OC)C(C)C)nc5s4)cc3)cc2)[nH]1)C(C)C]

or a pharmaceutically acceptable salt thereof.

Another embodiment of the invention a compound is of formula (IX):

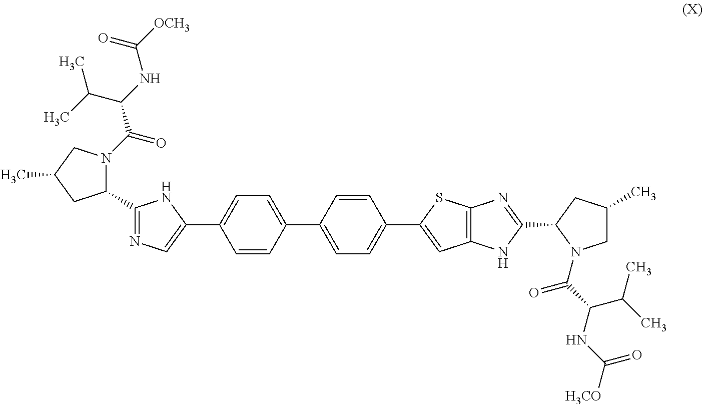 [SMILES: COC(=O)N[C@H](C(=O)N1C[C@@H](C)C[C@H]1c1ncc(-c2ccc(-c3ccc(-c4cc5[nH]c([C@@H]6C[C@H](C)CN6C(=O)[C@@H](NC(=O)OC)C(C)C)nc5s4)cc3)cc2)[nH]1)C(C)C]

or a pharmaceutically acceptable salt thereof.

The use of a compound of the present invention for treating an Hepatitis C viral infection in a human. The use of a compound of the present invention further comprising administering at least one additional agent. The use of a compound of the present invention wherein said at least one additional agent is selected from viral serine protease inhibitors, viral polymerase inhibitors, viral helicase inhibitors, immunomudulating agents, antioxidant agents, antibacterial agents, therapeutic vaccines, hepatoprotectant agents, antisense agents, inhibitors of HCV NS2/3 protease and inhibitors of internal ribosome entry site (IRES).

The use of a compound of the present invention, wherein said at least one additional agent is selected from ribavirin and interferon-α.

The use of a compound of the present invention for the manufacture of a medicament.

A pharmaceutical formulation comprising at least one compound of the present invention and at least one pharmaceutically acceptable carrier or excipient.

The use of a compound of the present invention for treating an Hepatitis C viral infection in a human. The use of a compound of the present invention further comprising administering at least one additional agent. The use of a compound of the present invention wherein said at least one additional agent is selected from viral serine protease inhibitors, viral polymerase inhibitors, viral helicase inhibitors, immunomudulating agents, antioxidant agents, antibacterial agents, therapeutic vaccines, hepatoprotectant agents, antisense agents, inhibitors of HCV NS2/3 protease and inhibitors of internal ribosome entry site (IRES). The use of a compound of the present invention wherein said at least one additional agent is selected from ribavirin and interferon-α.

The use of a compound of the present invention for the manufacture of a medicament.

A pharmaceutical formulation comprising at least one compound of the present invention and at least one pharmaceutically acceptable carrier or excipient.

According to an aspect of the invention, the compounds of the invention are selected from Tables 1A or a pharmaceutically acceptable salt thereof.

TABLE 1A

| Compound | Cmpd # |
|---|---|
| 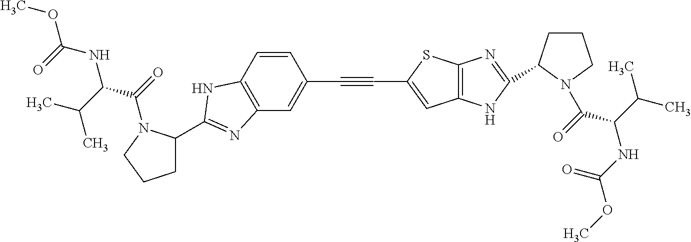 [SMILES: COC(=O)N[C@H](C(=O)N1CCCC1c1nc2cc(C#Cc3cc4[nH]c([C@@H]5CCCN5C(=O)[C@@H](NC(=O)OC)C(C)C)nc4s3)ccc2[nH]1)C(C)C] | 1 |
| 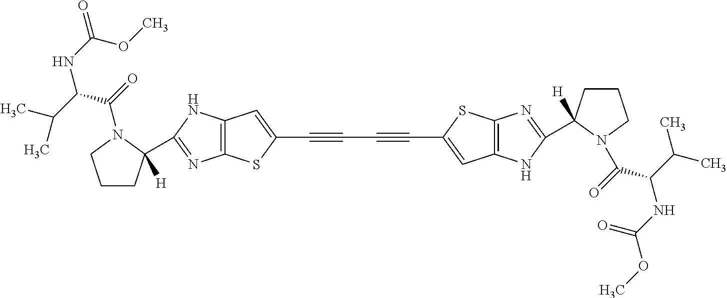 [SMILES: COC(=O)N[C@H](C(=O)N1CCC[C@H]1c1nc2sc(C#CC#Cc3cc4[nH]c([C@@H]5CCCN5C(=O)[C@@H](NC(=O)OC)C(C)C)nc4s3)cc2[nH]1)C(C)C] | 2 |
| 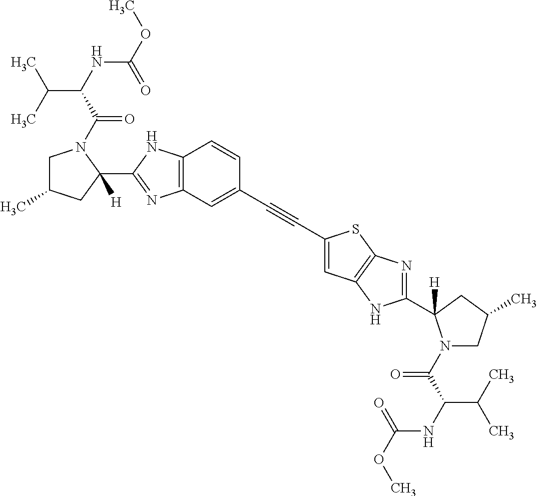 | 3 |
| 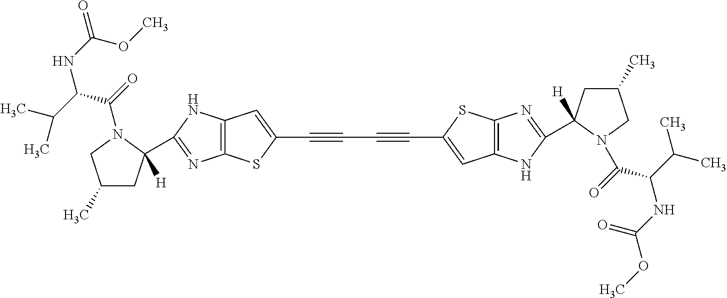 [SMILES: COC(=O)N[C@H](C(=O)N1C[C@@H](C)C[C@H]1c1nc2sc(C#CC#Cc3cc4[nH]c([C@@H]5C[C@H](C)CN5C(=O)[C@@H](NC(=O)OC)C(C)C)nc4s3)cc2[nH]1)C(C)C] | 4 |
| 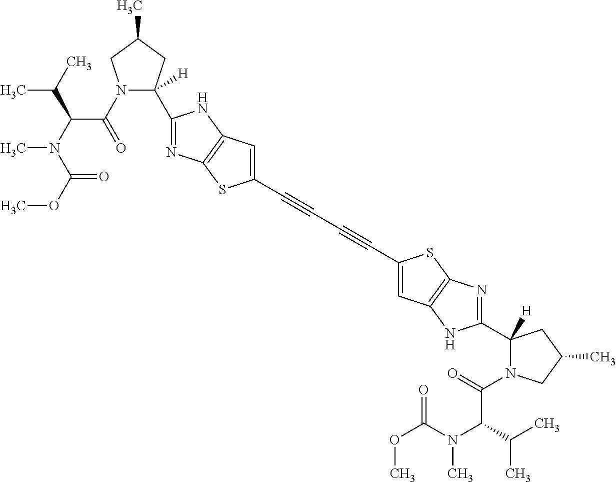 [SMILES: COC(=O)N(C)[C@H](C(=O)N1C[C@@H](C)C[C@H]1c1nc2sc(C#CC#Cc3cc4[nH]c([C@@H]5C[C@H](C)CN5C(=O)[C@H](C(C)C)N(C)C(=O)OC)nc4s3)cc2[nH]1)C(C)C] | 5 |
| 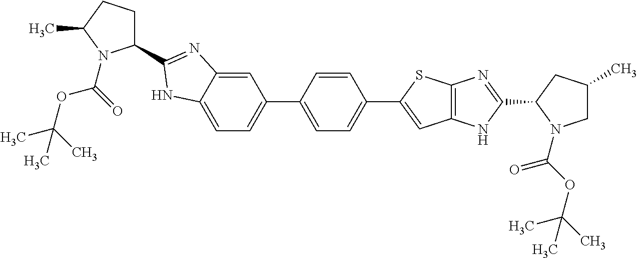 [SMILES: C[C@H]1C[C@@H](c2nc3sc(-c4ccc(-c5ccc6[nH]c([C@@H]7CC[C@H](C)N7C(=O)OC(C)(C)C)nc6c5)cc4)cc3[nH]2)N(C(=O)OC(C)(C)C)C1] | 6 |
| 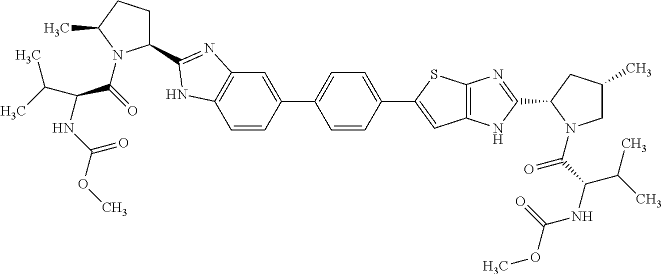 [SMILES: COC(=O)N[C@H](C(=O)N1C[C@@H](C)C[C@H]1c1nc2sc(-c3ccc(-c4ccc5[nH]c([C@@H]6CC[C@H](C)N6C(=O)[C@@H](NC(=O)OC)C(C)C)nc5c4)cc3)cc2[nH]1)C(C)C] | 7 |
| 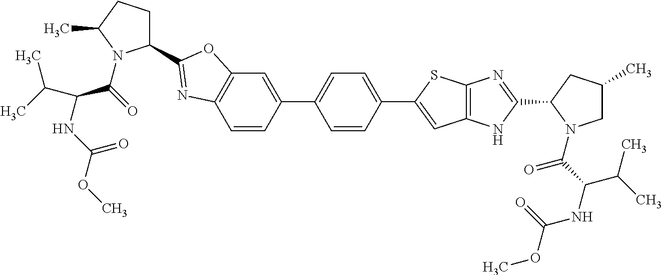 [SMILES: COC(=O)N[C@H](C(=O)N1C[C@@H](C)C[C@H]1c1nc2sc(-c3ccc(-c4ccc5nc([C@@H]6CC[C@H](C)N6C(=O)[C@@H](NC(=O)OC)C(C)C)oc5c4)cc3)cc2[nH]1)C(C)C] | 8 |
| 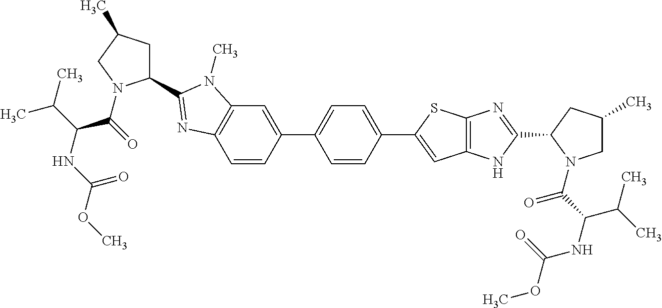 [SMILES: COC(=O)N[C@H](C(=O)N1C[C@@H](C)C[C@H]1c1nc2sc(-c3ccc(-c4ccc5nc([C@@H]6C[C@H](C)CN6C(=O)[C@@H](NC(=O)OC)C(C)C)n(C)c5c4)cc3)cc2[nH]1)C(C)C] | 9 |
| 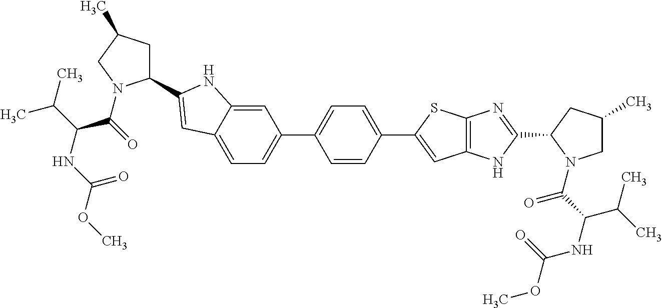 [SMILES: COC(=O)N[C@H](C(=O)N1C[C@@H](C)C[C@H]1c1cc2ccc(-c3ccc(-c4cc5[nH]c([C@@H]6C[C@H](C)CN6C(=O)[C@@H](NC(=O)OC)C(C)C)nc5s4)cc3)cc2[nH]1)C(C)C] | 10 |
| 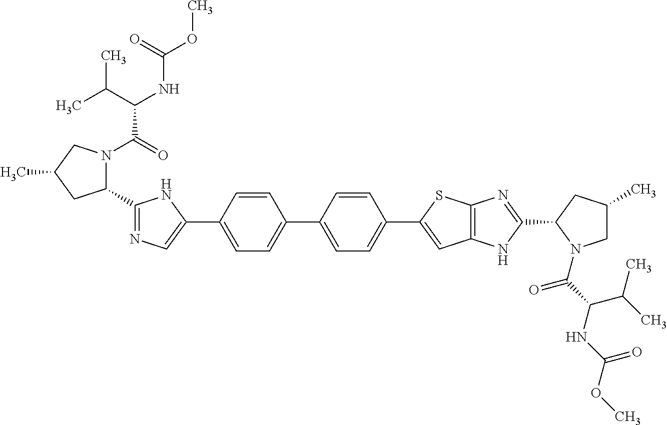 [SMILES: COC(=O)N[C@H](C(=O)N1C[C@@H](C)C[C@H]1c1ncc(-c2ccc(-c3ccc(-c4cc5[nH]c([C@@H]6C[C@H](C)CN6C(=O)[C@@H](NC(=O)OC)C(C)C)nc5s4)cc3)cc2)[nH]1)C(C)C] | 11 |
| 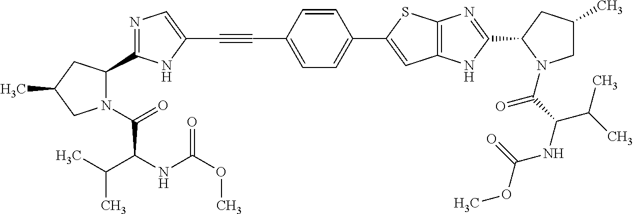 [SMILES: COC(=O)N[C@H](C(=O)N1C[C@@H](C)C[C@H]1c1ncc(C#Cc2ccc(-c3cc4[nH]c([C@@H]5C[C@H](C)CN5C(=O)[C@@H](NC(=O)OC)C(C)C)nc4s3)cc2)[nH]1)C(C)C] | 12 |
| 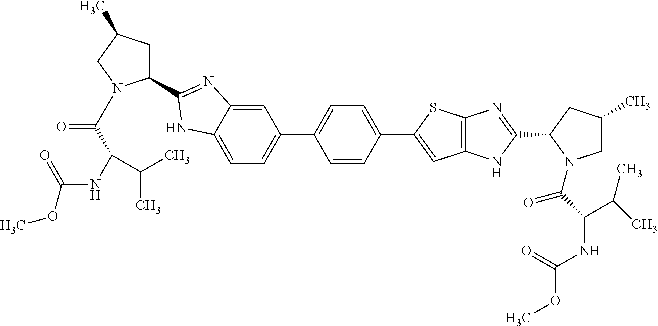 [SMILES: COC(=O)N[C@H](C(=O)N1C[C@@H](C)C[C@H]1c1nc2cc(-c3ccc(-c4cc5[nH]c([C@@H]6C[C@H](C)CN6C(=O)[C@@H](NC(=O)OC)C(C)C)nc5s4)cc3)ccc2[nH]1)C(C)C] | 13 |
| 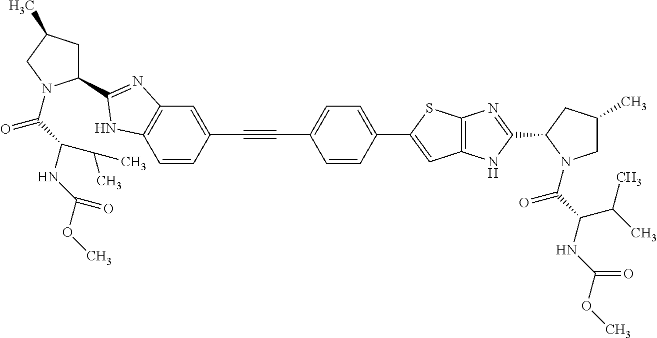 [SMILES: COC(=O)N[C@H](C(=O)N1C[C@@H](C)C[C@H]1c1nc2cc(C#Cc3ccc(-c4cc5[nH]c([C@@H]6C[C@H](C)CN6C(=O)[C@@H](NC(=O)OC)C(C)C)nc5s4)cc3)ccc2[nH]1)C(C)C] | 14 |
| 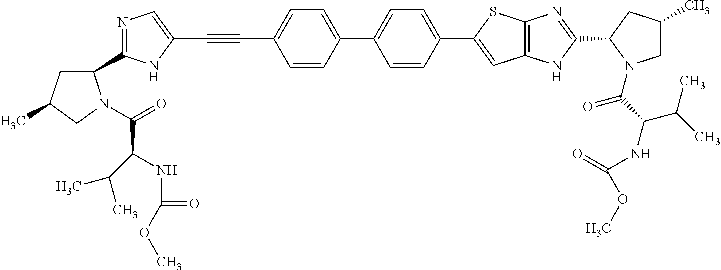 [SMILES: COC(=O)N[C@H](C(=O)N1C[C@@H](C)C[C@H]1c1ncc(C#Cc2ccc(-c3ccc(-c4cc5[nH]c([C@@H]6C[C@H](C)CN6C(=O)[C@@H](NC(=O)OC)C(C)C)nc5s4)cc3)cc2)[nH]1)C(C)C] | 15 |
| 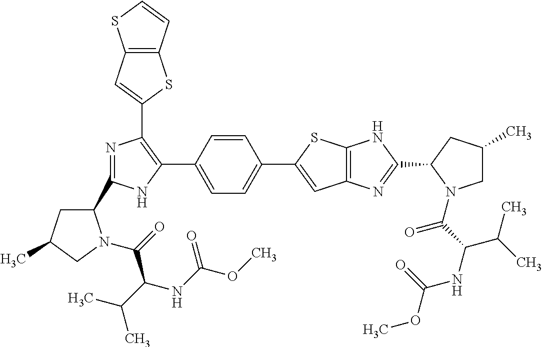 [SMILES: COC(=O)N[C@H](C(=O)N1C[C@@H](C)C[C@H]1c1nc(-c2cc3sccc3s2)c(-c2ccc(-c3cc4nc([C@@H]5C[C@H](C)CN5C(=O)[C@@H](NC(=O)OC)C(C)C)[nH]c4s3)cc2)[nH]1)C(C)C] | 16 |
| 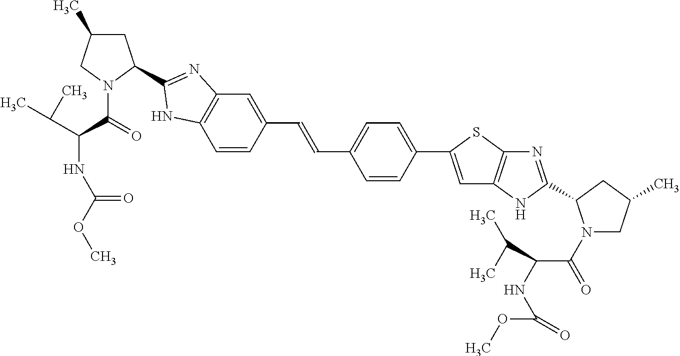 [SMILES: COC(=O)N[C@H](C(=O)N1C[C@@H](C)C[C@H]1c1nc2cc(/C=C/c3ccc(-c4cc5[nH]c([C@@H]6C[C@H](C)CN6C(=O)[C@@H](NC(=O)OC)C(C)C)nc5s4)cc3)ccc2[nH]1)C(C)C] | 17 |
| 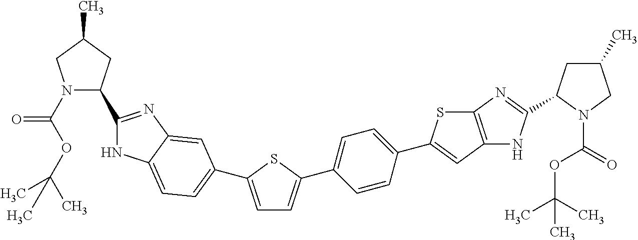 [SMILES: C[C@H]1C[C@@H](c2nc3cc(-c4ccc(-c5ccc(-c6cc7[nH]c([C@@H]8C[C@H](C)CN8C(=O)OC(C)(C)C)nc7s6)cc5)s4)ccc3[nH]2)N(C(=O)OC(C)(C)C)C1] | 18 |
| 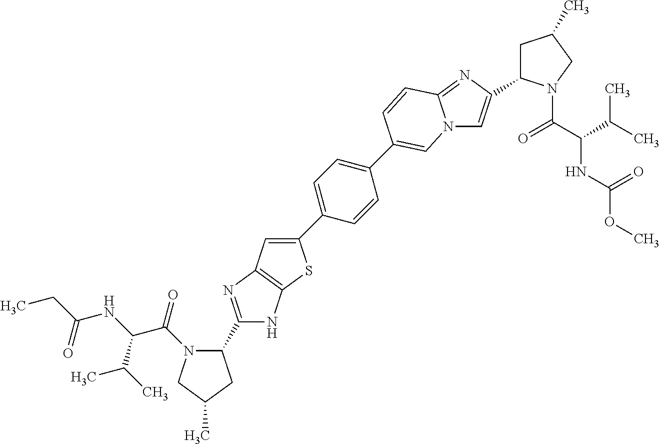 [SMILES: CCC(=O)N[C@H](C(=O)N1C[C@@H](C)C[C@H]1c1nc2cc(-c3ccc(-c4ccc5nc([C@@H]6C[C@H](C)CN6C(=O)[C@@H](NC(=O)OC)C(C)C)cn5c4)cc3)sc2[nH]1)C(C)C] | 19 |
| 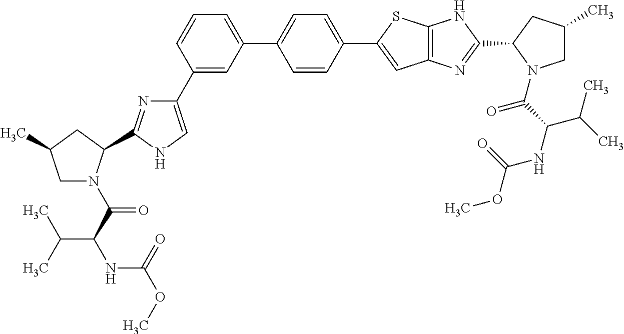 [SMILES: COC(=O)N[C@H](C(=O)N1C[C@@H](C)C[C@H]1c1nc(-c2cccc(-c3ccc(-c4cc5nc([C@@H]6C[C@H](C)CN6C(=O)[C@@H](NC(=O)OC)C(C)C)[nH]c5s4)cc3)c2)c[nH]1)C(C)C] | 20 |
| 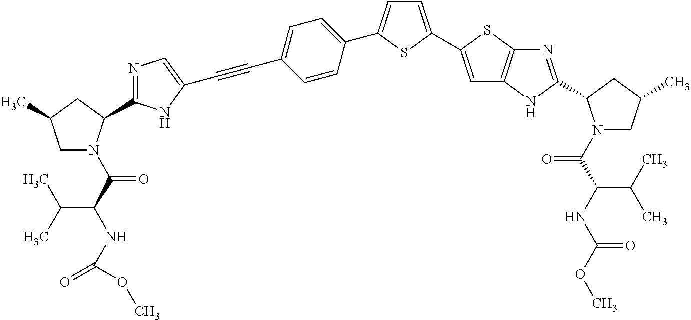 | 21 |
| 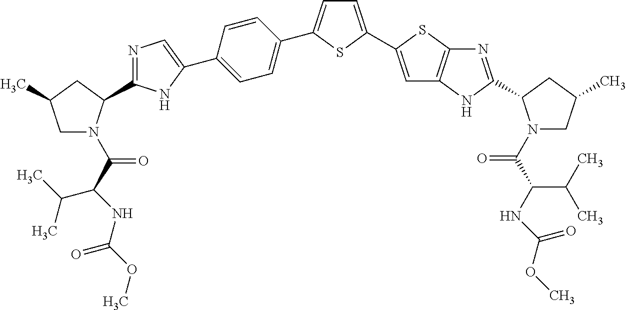 | 22 |
| 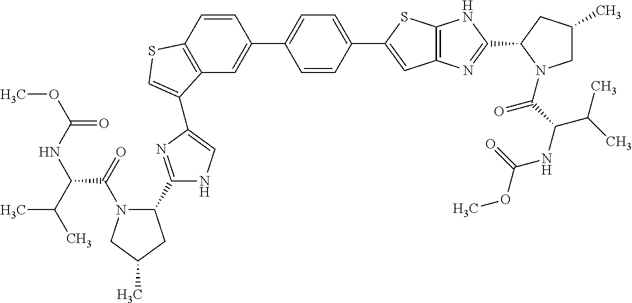 [SMILES: COC(=O)N[C@H](C(=O)N1C[C@@H](C)C[C@H]1c1nc(-c2csc3ccc(-c4ccc(-c5cc6nc([C@@H]7C[C@H](C)CN7C(=O)[C@@H](NC(=O)OC)C(C)C)[nH]c6s5)cc4)cc23)c[nH]1)C(C)C] | 23 |
| 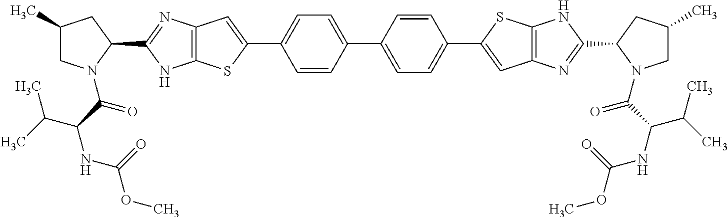 [SMILES: COC(=O)N[C@H](C(=O)N1C[C@@H](C)C[C@H]1c1nc2cc(-c3ccc(-c4ccc(-c5cc6nc([C@@H]7C[C@H](C)CN7C(=O)[C@@H](NC(=O)OC)C(C)C)[nH]c6s5)cc4)cc3)sc2[nH]1)C(C)C] | 24 |
| 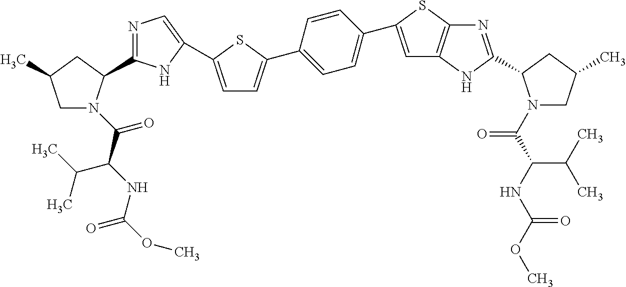 | 25 |
| 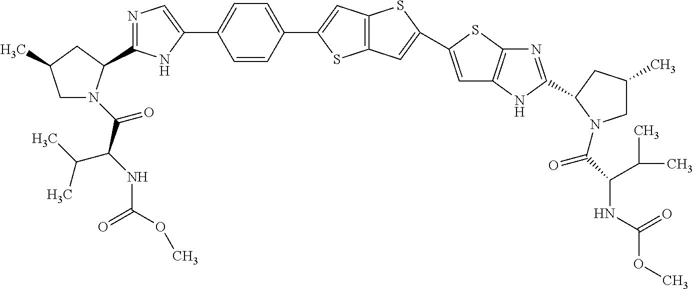 [SMILES: COC(=O)N[C@H](C(=O)N1C[C@@H](C)C[C@H]1c1ncc(-c2ccc(-c3cc4sc(-c5cc6[nH]c([C@@H]7C[C@H](C)CN7C(=O)[C@@H](NC(=O)OC)C(C)C)nc6s5)cc4s3)cc2)[nH]1)C(C)C] | 26 |
| 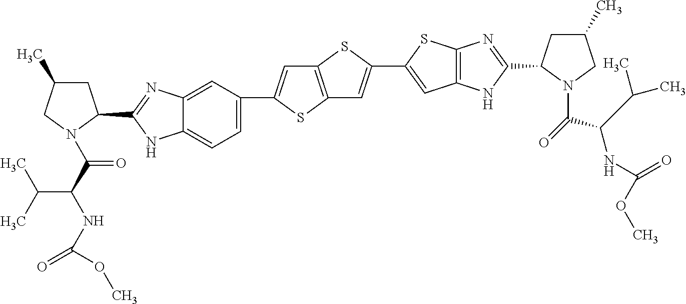 [SMILES: COC(=O)N[C@H](C(=O)N1C[C@@H](C)C[C@H]1c1nc2cc(-c3cc4sc(-c5cc6[nH]c([C@@H]7C[C@H](C)CN7C(=O)[C@@H](NC(=O)OC)C(C)C)nc6s5)cc4s3)ccc2[nH]1)C(C)C] | 27 |
| 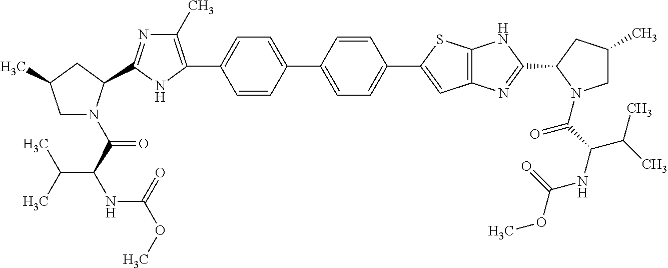 [SMILES: COC(=O)N[C@H](C(=O)N1C[C@@H](C)C[C@H]1c1nc(C)c(-c2ccc(-c3ccc(-c4cc5nc([C@@H]6C[C@H](C)CN6C(=O)[C@@H](NC(=O)OC)C(C)C)[nH]c5s4)cc3)cc2)[nH]1)C(C)C] | 28 |
| 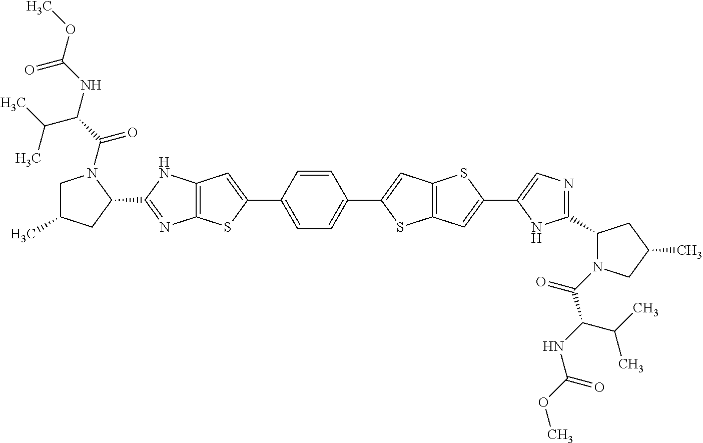 [SMILES: COC(=O)N[C@H](C(=O)N1C[C@@H](C)C[C@H]1c1ncc(-c2cc3sc(-c4ccc(-c5cc6[nH]c([C@@H]7C[C@H](C)CN7C(=O)[C@@H](NC(=O)OC)C(C)C)nc6s5)cc4)cc3s2)[nH]1)C(C)C] | 29 |
| 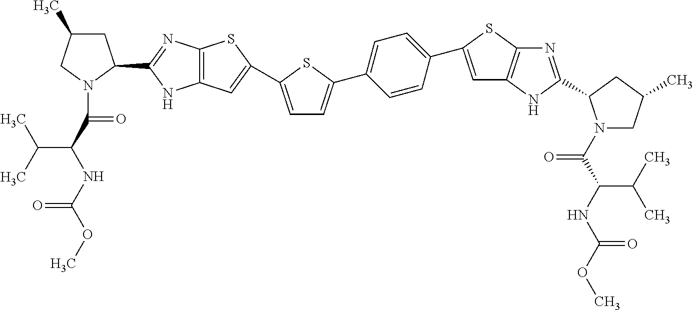 [SMILES: COC(=O)N[C@H](C(=O)N1C[C@@H](C)C[C@H]1c1nc2sc(-c3ccc(-c4ccc(-c5cc6[nH]c([C@@H]7C[C@H](C)CN7C(=O)[C@@H](NC(=O)OC)C(C)C)nc6s5)s4)cc3)cc2[nH]1)C(C)C] | 30 |
| 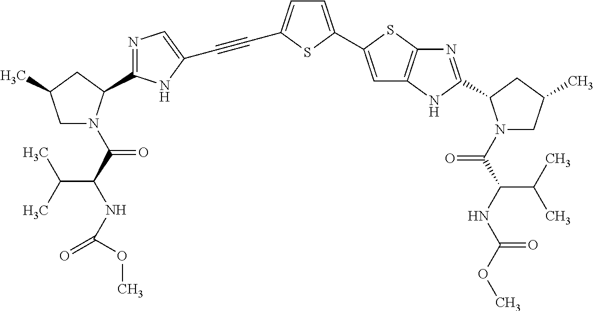 | 31 |
| 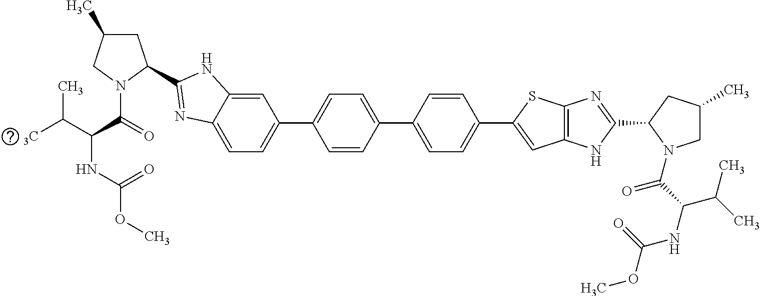 | 32 |

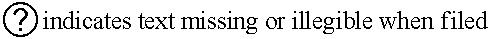

TABLE 1B

| Compound | Cmpd# |
|---|---|
| 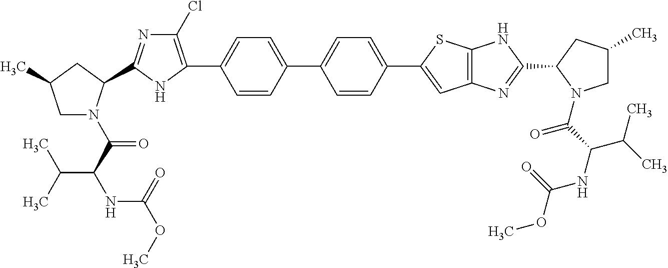 [SMILES: COC(=O)N[C@H](C(=O)N1C[C@@H](C)C[C@H]1c1nc(Cl)c(-c2ccc(-c3ccc(-c4cc5nc([C@@H]6C[C@H](C)CN6C(=O)[C@@H](NC(=O)OC)C(C)C)[nH]c5s4)cc3)cc2)[nH]1)C(C)C] | 1a |
| 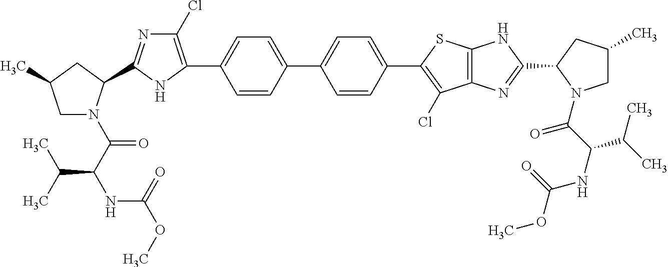 [SMILES: COC(=O)N[C@H](C(=O)N1C[C@@H](C)C[C@H]1c1nc(Cl)c(-c2ccc(-c3ccc(-c4sc5[nH]c([C@@H]6C[C@H](C)CN6C(=O)[C@@H](NC(=O)OC)C(C)C)nc5c4Cl)cc3)cc2)[nH]1)C(C)C] | 2a |
| 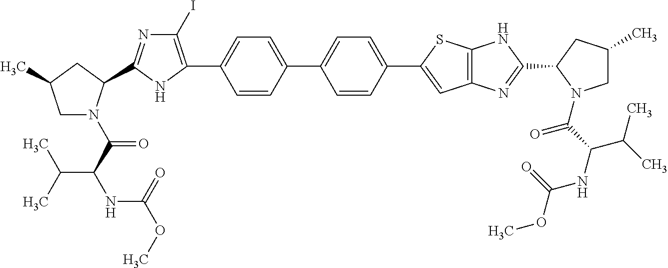 [SMILES: COC(=O)N[C@H](C(=O)N1C[C@@H](C)C[C@H]1c1nc(I)c(-c2ccc(-c3ccc(-c4cc5nc([C@@H]6C[C@H](C)CN6C(=O)[C@@H](NC(=O)OC)C(C)C)[nH]c5s4)cc3)cc2)[nH]1)C(C)C] | 3a |
| 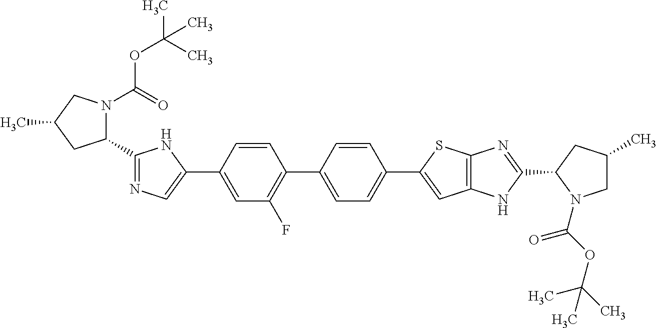 | 4a |
| 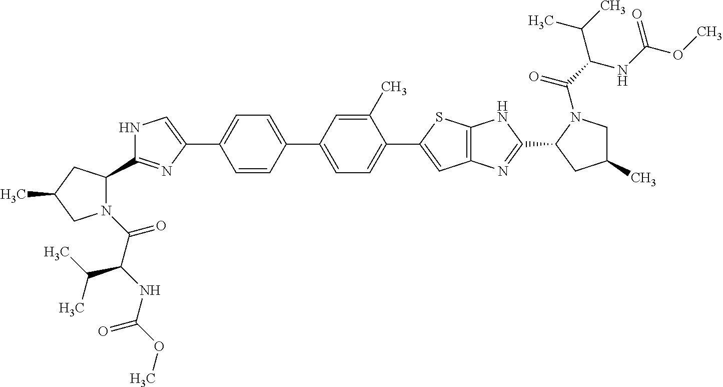 | 5a |
| 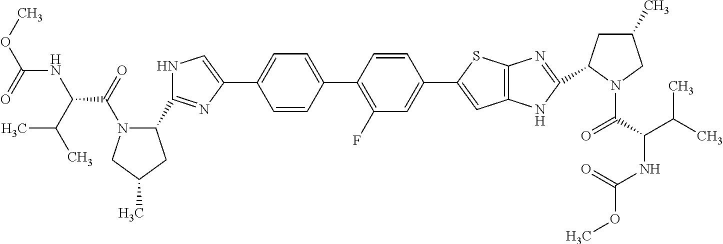 [SMILES: COC(=O)N[C@H](C(=O)N1C[C@@H](C)C[C@H]1c1nc(-c2ccc(-c3ccc(-c4cc5[nH]c([C@@H]6C[C@H](C)CN6C(=O)[C@@H](NC(=O)OC)C(C)C)nc5s4)cc3F)cc2)c[nH]1)C(C)C] | 6a |
| 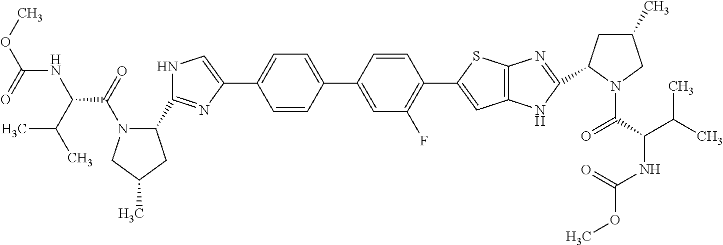 [SMILES: COC(=O)N[C@H](C(=O)N1C[C@@H](C)C[C@H]1c1nc(-c2ccc(-c3ccc(-c4cc5[nH]c([C@@H]6C[C@H](C)CN6C(=O)[C@@H](NC(=O)OC)C(C)C)nc5s4)c(F)c3)cc2)c[nH]1)C(C)C] | 7a |
| 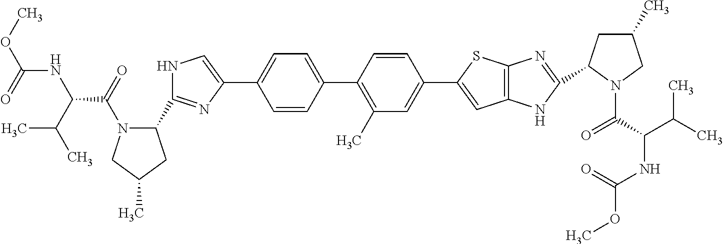 [SMILES: COC(=O)N[C@H](C(=O)N1C[C@@H](C)C[C@H]1c1nc(-c2ccc(-c3ccc(-c4cc5[nH]c([C@@H]6C[C@H](C)CN6C(=O)[C@@H](NC(=O)OC)C(C)C)nc5s4)cc3C)cc2)c[nH]1)C(C)C] | 8a |
| 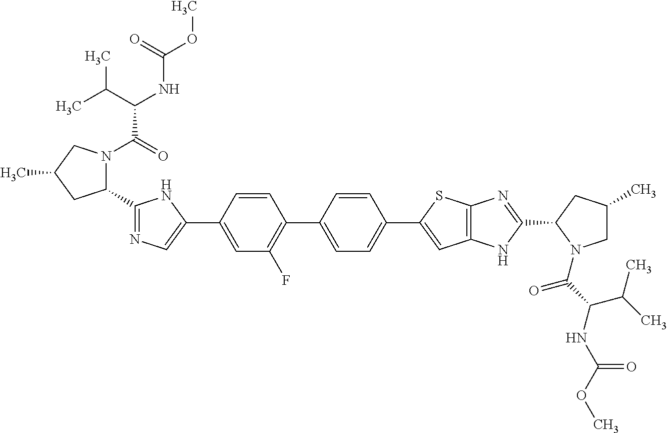 [SMILES: COC(=O)N[C@H](C(=O)N1C[C@@H](C)C[C@H]1c1ncc(-c2ccc(-c3ccc(-c4cc5[nH]c([C@@H]6C[C@H](C)CN6C(=O)[C@@H](NC(=O)OC)C(C)C)nc5s4)cc3)c(F)c2)[nH]1)C(C)C] | 9a |
| 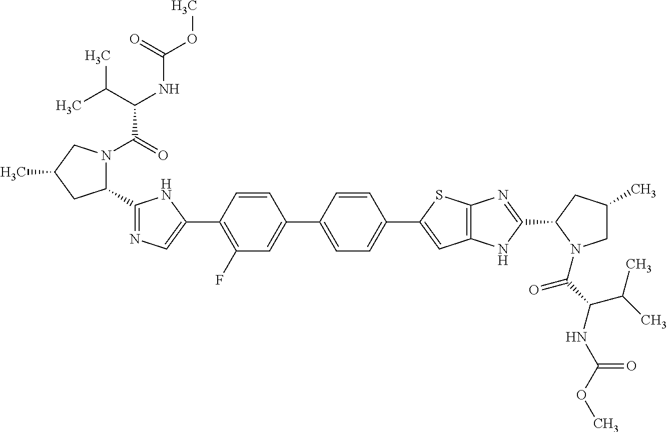 [SMILES: COC(=O)N[C@H](C(=O)N1C[C@@H](C)C[C@H]1c1ncc(-c2ccc(-c3ccc(-c4cc5[nH]c([C@@H]6C[C@H](C)CN6C(=O)[C@@H](NC(=O)OC)C(C)C)nc5s4)cc3)cc2F)[nH]1)C(C)C] | 10a |
| 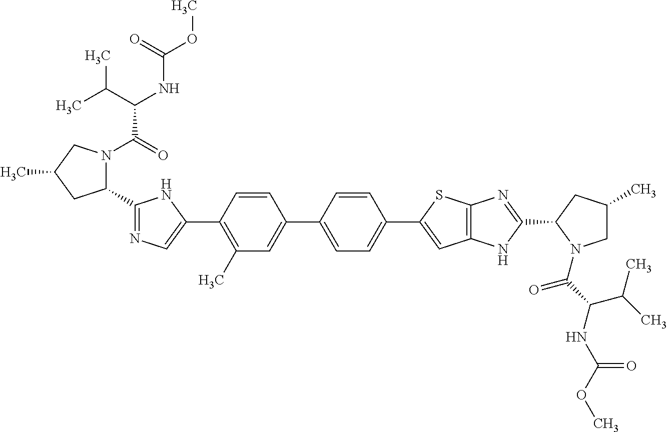 [SMILES: COC(=O)N[C@H](C(=O)N1C[C@@H](C)C[C@H]1c1ncc(-c2ccc(-c3ccc(-c4cc5[nH]c([C@@H]6C[C@H](C)CN6C(=O)[C@@H](NC(=O)OC)C(C)C)nc5s4)cc3)cc2C)[nH]1)C(C)C] | 11a |
| 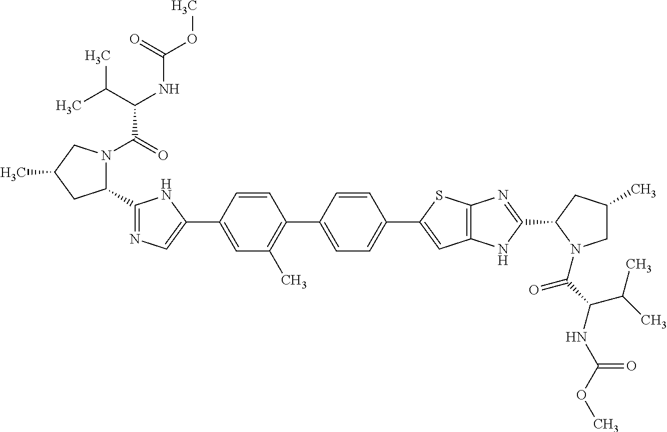 [SMILES: COC(=O)N[C@H](C(=O)N1C[C@@H](C)C[C@H]1c1ncc(-c2ccc(-c3ccc(-c4cc5[nH]c([C@@H]6C[C@H](C)CN6C(=O)[C@@H](NC(=O)OC)C(C)C)nc5s4)cc3)c(C)c2)[nH]1)C(C)C] | 12a |

and pharmaceutically acceptable salts thereof.

In one embodiment, the present invention is one or more of the compounds of Table 1A or a pharmaceutically acceptable salt thereof.

In one embodiment, the present invention is one or more of the compounds of Table 1B or a pharmaceutically acceptable salt thereof.

In one embodiment, the present invention provides a compound according to the invention described herein for treating or preventing a Flaviviridae viral infection in a host.

In one embodiment, the present invention provides a pharmaceutical composition comprising at least one compound according to the invention described herein and at least one pharmaceutically acceptable carrier or excipient.

In one embodiment, the present invention provides a pharmaceutical composition comprising at least one compound according to the invention described herein and at least one pharmaceutically acceptable carrier or excipient, for treating or preventing a Flaviviridae viral infection in a host.

In one embodiment, the present invention provides a pharmaceutical composition comprising at least one compound according to the invention described herein, and further comprising administering at least one additional agent chosen from viral serine protease inhibitors, viral polymerase inhibitors, viral helicase inhibitors, immunomudulating agents, antioxidant agents, antibacterial agents, therapeutic vaccines, hepatoprotectant agents, antisense agents, inhibitors of HCV NS2/3 protease and inhibitors of internal ribosome entry site (IRES).

In another embodiment, there is provided a combination comprising a least one compound according to the invention described herein and one or more additional agents.

In another embodiment, there is provided a combination comprising a least one compound according to the invention described herein and one or more additional agents chosen from viral serine protease inhibitors, viral polymerase inhibitors, viral helicase inhibitors, immunomudulating agents, antioxidant agents, antibacterial agents, therapeutic vaccines, hepatoprotectant agents, antisense agent, inhibitors of HCV NS2/3 protease and inhibitors of internal ribosome entry site (IRES).

In one combination embodiment, the compound and additional agent are administered sequentially.

In another combination embodiment, the compound and additional agent are administered simultaneously.

The combinations referred to above may conveniently be presented for use in the form of a pharmaceutical formulation and thus pharmaceutical formulations comprising a combination as defined above together with a pharmaceutically acceptable carrier therefore comprise a further aspect of the invention.

The additional agents for the compositions and combinations include, for example, ribavirin, amantadine, merimepodib, Levovirin, Viramidine, and maxamine.

The term “viral serine protease inhibitor” as used herein means an agent that is effective to inhibit the function of the viral serine protease including HCV serine protease in a mammal. Inhibitors of HCV serine protease include, for example, those compounds described in WO 99/07733 (Boehringer Ingelheim), WO 99/07734 (Boehringer Ingelheim), WO 00/09558 (Boehringer Ingelheim), WO 00/09543 (Boehringer Ingelheim), WO 00/59929 (Boehringer Ingelheim), WO 02/060926 (BMS), WO 2006039488 (Vertex), WO 2005077969 (Vertex), WO 2005035525 (Vertex), WO 2005028502 (Vertex) WO 2005007681 (Vertex), WO 2004092162 (Vertex), WO 2004092161 (Vertex), WO 2003035060 (Vertex), of WO 03/087092 (Vertex), WO 02/18369 (Vertex), or WO98/17679 (Vertex).

In one embodiment, the present invention provides a pharmaceutical composition comprising at least one compound according to the invention described herein, and further comprising one or more additional agents chosen from viral serine protease inhibitors, viral polymerase inhibitors, viral helicase inhibitors, immunomudulating agents, antioxidant agents, antibacterial agents, therapeutic vaccines, hepatoprotectant agents, antisense agent, inhibitors of HCV NS2/3 protease and inhibitors of internal ribosome entry site (IRES).

In another embodiment, there is provided a combination therapy of at least one compound according to the invention described herein in combination with one or more additional agents chosen from viral serine protease inhibitors, viral polymerase inhibitors, viral helicase inhibitors, immunomudulating agents, antioxidant agents, antibacterial agents, therapeutic vaccines, hepatoprotectant agents, antisense agent, inhibitors of HCV NS2/3 protease and inhibitors of internal ribosome entry site (IRES).

The additional agents for the compositions and combinations include, for example, ribavirin, amantadine, merimepodib, Levovirin, Viramidine, and maxamine.

In one combination embodiment, the compound and additional agent are administered sequentially.

In another combination embodiment, the compound and additional agent are administered simultaneously. The combinations referred to above may conveniently be presented for use in the form of a pharmaceutical formulation and thus pharmaceutical formulations comprising a combination as defined above together with a pharmaceutically acceptable carrier therefore comprise a further aspect of the invention.

The term “viral serine protease inhibitor” as used herein means an agent that is effective to inhibit the function of the viral serine protease including HCV serine protease in a mammal. Inhibitors of HCV serine protease include, for example, those compounds described in WO 99/07733 (Boehringer Ingelheim), WO 99/07734 (Boehringer Ingelheim), WO 00/09558 (Boehringer Ingelheim), WO 00/09543 (Boehringer Ingelheim), WO 00/59929 (Boehringer Ingelheim), WO 02/060926 (BMS), WO 2006039488 (Vertex), WO 2005077969 (Vertex), WO 2005035525 (Vertex), WO 2005028502 (Vertex) WO 2005007681 (Vertex), WO 2004092162 (Vertex), WO 2004092161 (Vertex), WO 2003035060 (Vertex), of WO 03/087092 (Vertex), WO 02/18369 (Vertex), or WO98/17679 (Vertex).

Specific examples of viral serine protease inhibitors include Telaprevir (VX-950, Vertex), VX-500 (Vertex), TMC435350 (Tibotec/Medivir), MK-7009 (Merck), ITMN-191 (R7227, InterMune/Roche) and Boceprevir (SCHSO3034, Schering).

The term “viral polymerase inhibitors” as used herein means an agent that is effective to inhibit the function of a viral polymerase including an HCV polymerase in a mammal. Inhibitors of HCV polymerase include non-nucleosides, for example, those compounds described in:

WO 03/010140 (Boehringer Ingelheim), WO 03/026587 (Bristol Myers Squibb); WO 02/100846 A1, WO 02/100851 A2, WO 01/85172 A1 (GSK), WO 02/098424 A1 (GSK), WO 00/06529 (Merck), WO 02/06246 A1 (Merck), WO 01/47883 (Japan Tobacco), WO 03/000254 (Japan Tobacco) and EP 1 256 628 A2 (Agouron).

Furthermore other inhibitors of HCV polymerase also include nucleoside analogs, for example, those compounds described in: WO 01/90121 A2 (Idenix), WO 02/069903 A2 (Biocryst Pharmaceuticals Inc.), and WO 02/057287 A2(Merck/Isis) and WO 02/057425 A2 (Merck/Isis).

Specific examples of inhibitors of an HCV polymerase, include VCH-759 (ViroChem Pharma), VCH-916 (ViroChem Pharma), VCH-222 (ViroChem Pharma), R1626 (Roche), R7128 (Roche/Pharmasset), PF-868554 (Pfizer), MK-0608 (Merck/Isis), MK-3281 (Merck), A-837093 (Abbott), GS 9190 (Gilead), ana598 (Anadys), HCV-796 (Viropharma) and GSK625433 (GlaxoSmithKline), R1479 (Roche), MK-0608 (Merck), R1656, (Roche-Pharmasset) and Valopicitabine (Idenix). Specific examples of inhibitors of an HCV polymerase, include JTK-002/003 and JTK-109 (Japan Tobacco), HCV-796 (Viropharma), GS-9190(Gilead), and PF-868,554 (Pfizer).

The term “viral helicase inhibitors” as used herein means an agent that is effective to inhibit the function of a viral helicase including a Flaviviridae helicase in a mammal.

“Immunomodulatory agent” as used herein means those agents that are effective to enhance or potentiate the immune system response in a mammal. Immunomodulatory agents include, for example, class I interferons (such as α-, β-, δ- and Ω-interferons, τ-interferons, consensus interferons and asialo-interferons), class II interferons (such as γ-interferons) and pegylated interferons.

Specific examples of Immunomodulatory agent as used herein include IL-29 (PEG-Interferon Lambda, ZymoGenetics), Belerofon (Nautilus Biotech) injectable or oral, Oral Interferon alpha (Amarillo Biosciences), BLX-883 (Locteron, Biolex Therapeutics/Octoplus), Omega Interferon (Intarcia Therapeutics), multiferon (Viragen), Albuferon (Human Genome Sciences), consensus Interferon (Infergen, Three Rivers Pharmaceuticals), Medusa Interferon (Flamel Technologies), NOV-205 (Novelos Therapeutics), Oglufanide disodium (Implicit Bioscience), SCV-07 (SciClone), Zadaxin® (thymalfasin, SciClone/Sigma-Tau), AB68 (XTL bio) and Civacir (NABI).

The term “viral polymerase inhibitors” as used herein means an agent that is effective to inhibit the function of a viral polymerase including an HCV polymerase in a mammal. Inhibitors of HCV polymerase include non-nucleosides, for example, those compounds described in: WO 03/010140 (Boehringer Ingelheim), WO 03/026587 (Bristol Myers Squibb); WO 02/100846 A1, WO 02/100851 A2, WO 01/85172 A1 (GSK), WO 02/098424 A1 (GSK), WO 00/06529 (Merck), WO 02/06246 A1 (Merck), WO 01/47883 (Japan Tobacco), WO 03/000254 (Japan Tobacco) and EP 1 256 628 A2 (Agouron).

Furthermore other inhibitors of HCV polymerase also include nucleoside analogs, for example, those compounds described in: WO 01/90121 A2 (Idenix), WO 02/069903 A2 (Biocryst Pharmaceuticals Inc.), and WO 02/057287 A2 (Merck/Isis) and WO 02/057425 A2 (Merck/lsis).

Specific examples of nucleoside inhibitors of an HCV polymerase, include R1626/R1479 (Roche), R7128 (Roche), MK-0608 (Merck), R1656, (Roche-Pharmasset) and Valopicitabine (Idenix). Specific examples of inhibitors of an HCV polymerase, include JTK-002/003 and JTK-109 (Japan Tobacco), HCV-796 (Viropharma), GS-9190(Gilead), and PF-868,554 (Pfizer).

The term “viral helicase inhibitors” as used herein means an agent that is effective to inhibit the function of a viral helicase including a Flaviviridae helicase in a mammal.

“Immunomodulatory agent” as used herein means those agents that are effective to enhance or potentiate the immune system response in a mammal. Immunomodulatory agents include, for example, class I interferons (such as alpha-, beta-, delta- and omega-interferons, x-interferons, consensus interferons and asialo-interferons), class II interferons (such as gamma-interferons) and pegylated interferons.

Exemplary immunomudulating agents, include, but are not limited to: thalidomide, IL-2, hematopoietins, IMPDH inhibitors, for example Merimepodib (Vertex Pharmaceuticals Inc.), interferon, including natural interferon (such as OMNIFERON, Viragen and SUMIFERON, Sumitomo, a blend of natural interferon's), natural interferon alpha (ALFERON, Hemispherx Biopharma, Inc.), interferon alpha n1 from lymphblastoid cells (WELLFERON, Glaxo Wellcome), oral alpha interferon, Peg-interferon, Peg-interferon alfa 2a (PEGASYS, Roche), recombinant interferon alpha 2a (ROFERON, Roche), inhaled interferon alpha 2b (AERX, Aradigm), Peg-interferon alpha 2b (ALBUFERON, Human Genome Sciences/Novartis, PEGINTRON, Schering), recombinant interferon alfa 2b (INTRON A, Schering), pegylated interferon alfa 2b (PEG-INTRON, Schering, VIRAFERONPEG, Schering), interferon beta-1a (REBIF, Serono, Inc. and Pfizer), consensus interferon alpha (INFERGEN, Valeant Pharmaceutical), interferon gamma-1b (ACTIMMUNE, Intermune, Inc.), un-pegylated interferon alpha, alpha interferon, and its analogs, and synthetic thymosin alpha 1 (ZADAXIN, SciClone Pharmaceuticals Inc.).

The term “class I interferon” as used herein means an interferon selected from a group of interferons that all bind to receptor type 1. This includes both naturally and synthetically produced class I interferons. Examples of class I interferons include α-, β-, δ- and Ω-interferons, τ-interferons, consensus interferons and asialo-interferons. The term “class II interferon” as used herein means an interferon selected from a group of interferons that all bind to receptor type II. Examples of class II interferons include γ-interferons.

Antisense agents include, for example, ISIS-14803.

Specific examples of inhibitors of HCV NS3 protease, include BILN-2061 (Boehringer Ingelheim) SCH-6 and SCH-503034/Boceprevir(Schering-Plough), VX-950/telaprevir(Vertex) and ITMN-B (InterMune), GS9132 (Gilead), TMC-435350(Tibotec/Medivir), ITMN-191 (InterMune), MK-7009 (Merck).

Inhibitors of internal ribosome entry site (IRES) include ISIS-14803 (ISIS Pharmaceuticals) and those compounds described in WO 2006019831 (PTC therapeutics).

In one embodiment, the additional agent is interferon α, ribavirin, silybum marianum, interleukine-12, amantadine, ribozyme, thymosin, N-acetyl cysteine or cyclosporin.

In one embodiment, the additional agent is interferon α, or ribavirin, silybum marianum, interleukine-12, amantadine, ribozyme, thymosin, N-acetyl cysteine or cyclosporin.

In one embodiment, the additional agent is interferon α 1A, interferon α 1B, interferon α 2A, or interferon α 2B.

Interferon is available in pegylated and non pegylated forms. Pegylated interferons include PEGASYS™ and Peg-intron™.

The recommended dose of PEGASYS™ monotherapy for chronic hepatitis C is 180 mg (1.0 mL vial or 0.5 mL prefilled syringe) once weekly for 48 weeks by subcutaneous administration in the abdomen or thigh.

The recommended dose of PEGASYS™ when used in combination with ribavirin for chronic hepatitis C is 180 mg (1.0 mL vial or 0.5 mL prefilled syringe) once weekly.

The recommended dose of PEG-Lntron™ regimen is 1.0 mg/kg/week subcutaneously for one year. The dose should be administered on the same day of the week.

When administered in combination with ribavirin, the recommended dose of PEG-Lntron is 1.5 micrograms/kg/week.

Ribavirin is typically administered orally, and tablet forms of ribavirin are currently commercially available. General standard, daily dose of ribavirin tablets (e.g., about 200 mg tablets) is about 800 mg to about 1200 mg. For example, ribavirn tablets are administered at about 1000 mg for subjects weighing less than 75 kg, or at about 1200 mg for subjects weighing more than or equal to 75 kg. Nevertheless, nothing herein limits the methods or combinations of this invention to any specific dosage forms or regime. Typically, ribavirin can be dosed according to the dosage regimens described in its commercial product labels.

In one embodiment, the additional agent is interferon α 1A, interferon α 1B, interferon α 2A (Roferon), PEG-interferon α 2A (Pegasys), interferon α 2B (Intron A) or PEG-interferon α 2B (Peg-Intron).

In one embodiment, the additional agent is standard or pegylated interferon α (Roferon, Pegasys, Intron A, Peg-Intron) in combination with ribavirin.

In one embodiment, the present invention provides a pharmaceutical composition comprising at least one compound according to the invention described herein, one or more additional agents select from non-nucleoside HCV polymerase inhibitors (e.g., HCV-796), nucleoside HCV polymerase inhibitors (e.g., R7128, R1626/R1479), HCV NS3 protease inhibitors (e.g., VX-950/telaprevir and ITMN-191), interferon and ribavirin, and at least one pharmaceutically acceptable carrier or excipient.

The combinations referred to above may conveniently be presented for use in the form of a pharmaceutical formulation and thus pharmaceutical formulations comprising a combination as defined above together with a pharmaceutically acceptable carrier therefore comprise a further aspect of the invention. The individual components for use in the method of the present invention or combinations of the present invention may be administered either sequentially or simultaneously in separate or combined pharmaceutical formulations.

In a further embodiment, the composition or combination according to the invention further comprises at least one compound according to the invention described herein; one or more additional agents select from non-nucleoside HCV polymerase inhibitors (e.g., HCV-796), nucleoside HCV polymerase inhibitors (e.g., R7128, R1626/R1479), and HCV NS3 protease inhibitors (e.g., VX-950/telaprevir and ITMN-191); and interferon and/or ribavirin.

In one embodiment, the additional agent is interferon α 1A, interferon α 1B, interferon α 2A, or interferon α 2B, and optionally ribavirin.

In one embodiment, the present invention provides a method for treating or preventing a HCV viral infection in a host comprising administering to the host a combined therapeutically effective amounts of at least one compound according to the invention described herein, and one or more additional agents select from non-nucleoside HCV polymerase inhibitors (e.g., HCV-796), nucleoside HCV polymerase inhibitors (e.g., R7128, R1626/R1479), HCV NS3 protease inhibitors (e.g., VX-950/telaprevir and ITMN-191), interferon and ribavirin.

In one combination embodiment, the compound and additional agent are administered sequentially.

In another combination embodiment, the compound and additional agent are administered simultaneously.

In one embodiment, there is provided a method for inhibiting or reducing the activity of HCV viral polymerase in a host comprising administering to the host a combined therapeutically effective amounts of at least one compound of the invention, and one or more additional agents select from non-nucleoside HCV polymerase inhibitors (e.g., HCV-796) and nucleoside HCV polymerase inhibitors (e.g., R7128, R1626/R1479), interferon and ribavirin.

In one embodiment, the present invention provides the use of at least one compound of the invention, in combination with the use of one or more additional agents select from non-nucleoside HCV polymerase inhibitors (e.g., HCV-796), nucleoside HCV polymerase inhibitors (e.g., R7128, R1626/R1479), HCV NS3 protease inhibitors (e.g., VX-950/telaprevir and ITMN-191), interferon and ribavirin, for the manufacture of a medicament for treating or preventing a HCV infection in a host.

When the compounds of the invention described herein are used in combination with at least one second therapeutic agent active against the same virus, the dose of each compound may be either the same as or differ from that when the compound is used alone. Appropriate doses will be readily appreciated by those skilled in the art.

The ratio of the amount of a compound according to the invention described herein administered relative to the amount of the additional agent (non-nucleoside HCV polymerase inhibitors (e.g., HCV-796), nucleoside HCV polymerase inhibitors (e.g., R7128, R1626/R1479), HCV NS3 protease inhibitors (e.g., VX-950/telaprevir and ITMN-191), interferon or ribavirin) will vary dependent on the selection of the compound and additional agent.

In one embodiment, the additional agent is chosen from A-831 (AZD0530, Arrow Therapeutics acquired by AstraZeneca), TLR9 agonist: IMO-2125 (Idera Pharmaceuticals), PYN17 (Phynova), Vavituximab (Tarvacin, Peregrine), DEBIO-025 (DEBIO), NIM-811 (Novartis), SCY635 (Scynexis), PF-03491390 (IDN-6556, Pfizer), Suvus (formerly BIVN-401, Virostat, Bioenvision), MX-3253 (Celgosivir, Migenix), Viramidine (Taribavirin, Valeant Pharmaceuticals), Hepaconda (Giaconda), TT033 (Benitec/Tacere Bio/Pfizer), SIRNA-034 (Sirna Therapeutics aquired by Merck) and EHC-18 (Enzo Biochem), ACH-1095 (Achillion/Gilead), JKB-022 (Jenkin), CTS-1027 (Conatus), MitoQ (mitoquinone, Antipodean Pharmaceuticals), Alinia (nitazoxanide, Romark Laboratories) and Bavituximab (Peregrine Pharm).

In one embodiment, the additional agent is a therapeutic vaccine chosen from CSL123 (Chiron/CSL), IC41 (Intercell Novartis), GI 5005 (Globeimmune), TG4040 (Transgene), Chronvac C (Tripep/Inovio), GNI-103 (GENimmune), HCV/MF59 (Chiron/Novartis), PeviPRO™ (Pevion biotect).

The recommended dose of PEGASYS™ monotherapy for chronic hepatitis C is 180 mg (1.0 mL vial or 0.5 mL prefilled syringe) once weekly for 48 weeks by subcutaneous administration in the abdomen or thigh.

In one embodiment, viral serine protease inhibitor is a flaviviridae serine protease inhibitor.

In one embodiment, viral polymerase inhibitor is a flaviviridae polymerase inhibitor.

In one embodiment, viral helicase inhibitor is a flaviviridae helicase inhibitor.

In further embodiments:

viral serine protease inhibitor is HCV serine protease inhibitor;

viral polymerase inhibitor is HCV polymerase inhibitor;

viral helicase inhibitor is HCV helicase inhibitor.

In one embodiment, the present invention provides a method for treating or preventing a Flaviviridae viral infection in a host comprising administering to the host a therapeutically effective amount of at least one compound according to formula (I), (II), (III), or (IV).

In one embodiment, the viral infection is chosen from Flavivirus infections.

In one embodiment, the Flavivirus infection is Hepatitis C virus (HCV), bovine viral diarrhea virus (BVDV), hog cholera virus, dengue fever virus, Japanese encephalitis virus or yellow fever virus.

In one embodiment, the Flaviviridea viral infection is hepatitis C viral infection (HCV).

In one embodiment, the host is human.

In one embodiment, the present invention provides a method for treating or preventing a Flaviviridae viral infection in a host comprising administering to the host a therapeutically effective amount of at least one compound according to the invention described herein, and further comprising administering at least one additional agent.

In one embodiment, the present invention provides a method for treating or preventing a Flaviviridae viral infection in a host comprising administering to the host a therapeutically effective amount of at least one compound according to the invention described herein, and further comprising administering at least one additional agent chosen from viral serine protease inhibitors, viral polymerase inhibitors, viral helicase inhibitors, immunomudulating agents, antioxidant agents, antibacterial agents, therapeutic vaccines, hepatoprotectant agents, antisense agents, inhibitors of HCV NS2/3 protease and inhibitors of internal ribosome entry site (IRES).

The combinations referred to above may conveniently be presented for use in the form of a pharmaceutical formulation and thus pharmaceutical formulations comprising a combination as defined above together with a pharmaceutically acceptable carrier therefore comprise a further aspect of the invention.

The individual components for use in the method of the present invention or combinations of the present invention may be administered either sequentially or simultaneously in separate or combined pharmaceutical formulations.

In one embodiment, the present invention provides the use of a compound according to the invention described herein for treating or preventing Flaviviridae viral infection in a host.

In one embodiment, the present invention provides the use of a compound according to the invention described herein and further comprising at least one additional agent chosen from viral serine protease inhibitors, viral polymerase inhibitors, viral helicase inhibitors, immunomudulating agents, antioxidant agents, antibacterial agents, therapeutic vaccines, hepatoprotectant agents, antisense agents, inhibitors of HCV NS2/3 protease and inhibitors of internal ribosome entry site (IRES). for treating or preventing Flaviviridae viral infection in a host.

In one embodiment, the present invention provides the use of a compound according to the invention described herein for the manufacture of a medicament.

In one embodiment, the present invention provides the use of a compound according to the invention described herein for the manufacture of a medicament for treating or preventing a viral Flaviviridae infection in a host.

In one embodiment, the present invention provides the use of a compound according to the invention described herein and further comprising at least one additional agent chosen from viral serine protease inhibitors, viral polymerase inhibitors, viral helicase inhibitors, immunomudulating agents, antioxidant agents, antibacterial agents, therapeutic vaccines, hepatoprotectant agents, antisense agents, inhibitors of HCV NS2/3 protease and inhibitors of internal ribosome entry site (IRES). for the manufacture of a medicament for treating or preventing a viral Flaviviridae infection in a host.

In one embodiment, the present invention provides a method of treating or preventing infection by a HCV virus, comprising contacting a biological sample or administering to a patient in need thereof a compound disclosed herein in an amount effective to treat or prevent the infection.

In one embodiment of the method, HCV is of genotype 1. In another embodiment, HCV is of genotype 1a, genotype 1b, or a combination thereof.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. The single optical isomer or enantiomer can be obtained by method well known in the art, such as chiral HPLC, enzymatic resolution and chiral auxiliary.

Unless otherwise stated, all tautomeric forms of the compounds of the invention are within the scope of the invention.

In one embodiment, the compounds of the present invention are provided in the form of a single stereoisomer at least 95%, at least 97% and at least 99% free of the corresponding stereoisomers.

In a further embodiment the compound of the present invention are in the form of a single stereoisomer at least 95% free of the corresponding stereoisomers.

In a further embodiment the compound of the present invention are in the form of a single stereoisomer at least 97% free of the corresponding stereoisomers.

In a further embodiment the compound of the present invention are in the form of a single stereoisomer at least 99% free of the corresponding stereoisomers.

There is also provided pharmaceutically acceptable salts of the compounds of the present invention. By the term pharmaceutically acceptable salts of compounds are meant those derived from pharmaceutically acceptable inorganic and organic acids and bases. Examples of suitable acids include hydrochloric, hydrobromic, sulphuric, nitric, perchloric, fumaric, maleic, phosphoric, glycollic, lactic, salicylic, succinic, toleune-p-sulphonic, tartaric, acetic, trifluoroacetic, citric, methanesulphonic, formic, benzoic, malonic, naphthalene-2-sulphonic and benzenesulphonic acids. Other acids such as oxalic, while not themselves pharmaceutically acceptable, may be useful as intermediates in obtaining the compounds of the invention and their pharmaceutically acceptable acid addition salts.

Salts derived from amino acids are also included (e.g. L-arginine, L-Lysine).

Salts derived from appropriate bases include alkali metals (e.g. sodium, lithium, potassium) and alkaline earth metals (e.g. calcium, magnesium).

A reference hereinafter to a compound according to the invention includes that compound and its pharmaceutically acceptable salts.

With regards to pharmaceutically acceptable salts, see also the list of FDA approved commercially marketed salts listed in Table I of Berge et al., Pharmaceutical Salts, J. of Phar. Sci., vol. 66, no. 1, January 1977, pp. 1-19, the disclosure of which is incorporated herein by reference.

It will be appreciated by those skilled in the art that the compounds in accordance with the present invention can exist in different polymorphic forms. As known in the art, polymorphism is an ability of a compound to crystallize as more than one distinct crystalline or “polymorphic” species. A polymorph is a solid crystalline phase of a compound with at least two different arrangements or polymorphic forms of that compound molecule in the solid state. Polymorphic forms of any given compound are defined by the same chemical formula or composition and are as distinct in chemical structure as crystalline structures of two different chemical compounds.

It will further be appreciated by those skilled in the art that the compounds in accordance with the present invention can exist in different solvate forms, for example hydrates. Solvates of the compounds of the invention may also form when solvent molecules are incorporated into the crystalline lattice structure of the compound molecule during the crystallization process.

In addition to the compounds of this invention, pharmaceutically acceptable derivatives or prodrugs, and esters, of the compounds of this invention may also be employed in compositions to treat or prevent the herein identified disorders. Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

In the formulas and drawings, a line transversing a ring and bonded to a group such as B, B′, R₁, R₄ or R₄′ in formula (I)

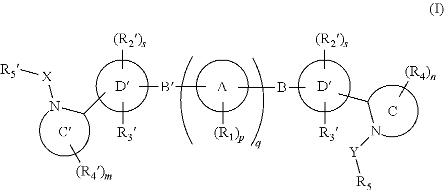

means that the group can be bonded to any carbon, or if applicable, heteroatom such as N, of that ring as valency allows.

The term “alkyl” represents a linear, branched or cyclic hydrocarbon moiety. The terms “alkenyl” and “alkynyl” represent a linear, branched or cyclic hydrocarbon moiety which has one or more double bonds or triple bonds in the chain. Examples of alkyl, alkenyl, and alkynyl groups include but are not limited to methyl, ethyl, propyl, isopropyl, butyl, isobutyl, sec-butyl, tert-butyl, pentyl, isopentyl, neopentyl, tert-pentyl, hexyl, isohexyl, neohexyl, allyl, vinyl, acetylenyl, ethylenyl, propenyl, isopropenyl, butenyl, isobutenyl, hexenyl, butadienyl, pentenyl, pentadienyl, hexenyl, heptenyl, heptadienyl, heptatrienyl, octenyl, propynyl, butynyl, pentynyl, hexynyl, cyclopropyl, cyclobutyl, cyclohexenyl, cyclohexdienyl and cyclohexyl. The terms alkyl, alkenyl, and alkynyl, also include combinations of linear and branched groups, e.g., cyclopropylmethyl, cyclohexylethyl, etc. The term alkenyl also includes C1 alkenyl where the one carbon atom is attached to the remainder of the molecule via a double bond. Where indicated the “alkyl,” “alkenyl,” and “alkynyl” can be optionally substituted such as in the case of haloalkyls in which one or more hydrogen atom is replaced by a halogen, e.g., an alkylhalide. Examples of haloalkyls include but are not limited to trifluoromethyl, difluoromethyl, fluoromethyl, trichloromethyl, dichloromethyl, chloromethyl, trifluoroethyl, difluoroethyl, fluoroethyl, trichloroethyl, dichloroethyl, chloroethyl, chlorofluoromethyl, chlorodifluoromethyl, dichlorofluoroethyl. Aside from halogens, where indicated, the alkyl, alkenyl or alkynyl groups can also be optionally substituted by, for example, halogen, —ORₐ, oxo, —NRₐR_{b}, ═NO—R_{c}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, wherein Rₐ—R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

The terms “cycloalkyl”, and “cycloalkenyl” represent a cyclic hydrocarbon alkyl or alkenyl, respectively, and are meant to include monocyclic (e.g., cyclopropyl, cyclobutyl, cyclohexyl), spiro (e.g., spiro[2.3]hexanyl), fused (e.g., bicyclo[4.4.0]decanyl), and bridged (e.g., bicyclo[2.2.1]heptanyl)hydrocarbon moieties.

The terms “alkoxy,” “alkenyloxy,” and “alkynyloxy” represent an alkyl, alkenyl or alkynyl moiety, respectively, which is covalently bonded to the adjacent atom through an oxygen atom. Examples include but are not limited to methoxy, ethoxy, propoxy, isopropoxy, butoxy, isobutoxy, sec-butoxy, tert-butoxy, pentyloxy, isopentyloxy, neopentyloxy, tert-pentyloxy, hexyloxy, isohexyloxy, trifluoromethoxy and neohexyloxy. Like the alkyl, alkenyl and alkynyl groups, where indicated the alkoxy, alkenyloxy, and alkynyloxy groups can be optionally substituted by, for example, halogen, —ORₐ, oxo, —NRₐR_{b}, ═NO—R_{c}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, wherein Rₐ—R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

The term “aryl” represents a carbocyclic moiety containing at least one benzenoid-type ring (i.e., may be monocyclic or polycyclic), and which where indicated may be optionally substituted with one or more substituents. Examples include but are not limited to phenyl, tolyl, dimethylphenyl, aminophenyl, anilinyl, naphthyl, anthryl, phenanthryl or biphenyl. The aryl groups can be optionally substituted where indicated by, for example, halogen, —ORₐ, —NRₐR_{b}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl, wherein Rₐ—R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

The term “aralkyl” represents an aryl group attached to the adjacent atom by an alkyl, alkenyl or alkynyl. Like the aryl groups, where indicated the aralkyl groups can also be optionally substituted. Examples include but are not limited to benzyl, benzhydryl, trityl, phenethyl, 3-phenylpropyl, 2-phenylpropyl, 4-phenylbutyl and naphthylmethyl. Where indicated, the aralkyl groups can be optionally substituted one or more times by, for example, halogen, —ORₐ, —NRₐR_{b}, —C(═O)ORₐ, —C(O)NRₐR_{b}, —C(═O)OH, —C(═O)Rₐ, —C(═NOR_{c})Rₐ, —C(═NR_{c′})NRₐR_{b}, —NR_{d}C(═O)NRₐR_{b}, —NR_{b}C(═O)Rₐ, —NR_{d}C(═NR_{c′})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl, wherein Rₐ—R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

The term “heterocycle” represents a non aromatic, saturated or partially saturated cyclic moiety wherein said cyclic moiety is interrupted by at least one heteroatom selected from oxygen (O), sulfur (S) or nitrogen (N). Heterocycles may be monocyclic or polycyclic rings. Examples include but are not limited to azetidinyl, dioxolanyl, morpholinyl, morpholino, oxetanyl, piperazinyl, piperidyl, piperidinyl, cyclopentapyrazolyl, cyclopentaoxazinyl, cyclopentafuranyl, tetrahydrofuranyl, thiazolinyl, oxazolinyl, pyranyl, aziridinyl, azepinyl, dioxazepinyl, diazepinyl, oxyranyl, oxazinyl, pyrrolidinyl, and thiopyranyl, thiolanyl, pyrazolidinyl, dioxanyl, and imidazolidinyl. Where indicated, the heterocyclic groups can be optionally substituted one or more times by, for example, halogen, —ORₐ, oxo, —NRₐR_{b}, ═NO—R_{c}, —C(═O)ORₐ, —C(O)NRₐR_{b}, —C(═O)OH, —C(═O)Rₐ, —C(═NOR_{c})Rₐ, —C(═NR_{c′})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl, wherein Rₐ—R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

The term “heterocycle-alkyl” represents a heterocycle group attached to the adjacent atom by an alkyl, alkenyl or alkynyl group. It is understood that in, for example, a 4-18 member heterocycle-alkyl moiety, the 4-18 member represent the total of the ring atoms present in the heterocycle moiety and the carbon atoms present in the alkyl, alkenyl or alkynyl group. For example, the following groups are encompassed by a 7 member heterocycle-alkyl (* represents the attachment point):

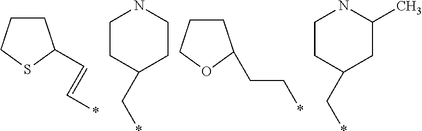

Where indicated the heterocycle-alkyl groups can be optionally substituted one or more times by, for example, halogen, —ORₐ, oxo, —NRₐR_{b}, ═NO—R_{c}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl, wherein Rₐ—R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

The term “heteroaryl” represents an aromatic cyclic moiety wherein said cyclic moiety is interrupted by at least one heteroatom selected from oxygen (O), sulfur (S) or nitrogen (N). Heteroaryls may be monocyclic or polycyclic rings wherein at least one ring in the polycyclic ring system is aromatic and at least one ring (not necessarily the same ring contains a heteroatom. Examples include but are not limited to dithiadiazinyl, furanyl, isooxazolyl, isothiazolyl, imidazolyl, oxadiazolyl, oxazolyl, pyrazinyl, pyridazinyl, pyrimidinyl, pyridyl, pyrazolyl, pyrrolyl, thiatriazolyl, tetrazolyl, thiadiazolyl, triazolyl, thiazolyl, thienyl, tetrazinyl, thiadiazinyl, triazinyl, thiazinyl, furoisoxazolyl, imidazothiazolyl, thienoisothiazolyl, thienothiazolyl, imidazopyrazolyl, pyrrolopyrrolyl, thienothienyl, thiadiazolopyrimidinyl, thiazolothiazinyl, thiazolopyrimidinyl, thiazolopyridinyl, oxazolopyrimidinyl, oxazolopyridyl, benzoxazolyl, benzisothiazolyl, benzothiazolyl, benzodioxolyl, dihydrobenzodioxinyl, benzothiadiazolyl, thienofuranyl, imidazopyrazinyl, purinyl, pyrazolopyrimidinyl, imidazopyridinyl, benzimidazolyl, indazolyl, benzoxathiolyl, benzodioxolyl, benzodithiolyl, indolizinyl, indolinyl, isoindolinyl, furopyrimidinyl, furopyridyl, benzofuranyl, isobenzofuranyl, thienopyrimidinyl, thienopyridyl, benzothienyl, benzoxazinyl, benzothiazinyl, quinazolinyl, naphthyridinyl, quinolinyl, isoquinolinyl, benzopyranyl, pyridopyridazinyl, chromen, benzodiazinyl. Where indicated the heteroaryl groups can be optionally substituted one or more times by, for example, halogen, —ORₐ, —NRₐR_{b}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c′})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl, wherein Rₐ—R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

The term “heteroaralkyl” represents an optionally substituted heteroaryl group attached to the adjacent atom by an alkyl, alkenyl or alkynyl group. Where indicated the heteroaralkyl groups can be optionally substituted one or more times by, for example, halogen, —ORₐ, —NRₐR_{b}, —C(═O)ORₐ, —C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl, wherein Rₐ—R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl. It is understood that in, for example, a 6-18 member heteroaralkyl moiety, the 6-18 member represents the total of the ring atoms present in the heterocycle moiety and the carbon atoms in the alkyl, alkenyl or alkynyl groups. For example, the following groups are encompassed by a 7 member heteroaralkyl (* represents the attachment point):

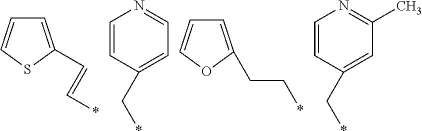

“Halogen atom or halo” is specifically a fluorine atom, chlorine atom, bromine atom or iodine atom.

The term “oxo” represents ═O.

A dash (“-”) that is not between two letters or symbols is used to indicate a point of attachement for a substitutent. For example, —CONR_{d}Rₑ is attached through the carbon of the amide.

A dash line (“-----”) is used to indicate the point of attachment for the group. For example, A is attached through the carbon at position 1 and 4 in the following representation:

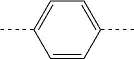

When there is a sulfur atom present, the sulfur atom can be at different oxidation levels, i.e., S, SO, or SO₂. All such oxidation levels are within the scope of the present invention.

The term “independently” means that a substituent can be the same or a different definition for each item.

In general, the term “substituted,” whether preceded by the term “optionally” or not, refers to the replacement of hydrogen radicals on a carbon or nitrogen atom in a given structure with the radical of a specified substituent. Specific substituents are described above in the definitions and below in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. For example, the language, “which is unsubstituted or substituted one or more times by R¹⁰” means that when the group is substituted with more than one R¹⁰ group, the R¹⁰ groups can be different from each other. A ring substituent, such as a heterocycle, can be bound to another ring, such as a cycloalkyl, to form a spiro-bicyclic ring system, e.g., both rings share one common atom.

As one of ordinary skill in the art will recognize, combinations of substituents envisioned by this invention are those combinations that result in the formation of stable or chemically feasible compounds. The term “stable”, as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week. When two alkoxy groups are bound to the same atom or adjacent atoms, the two alkoxy groups can form a ring together with the atom(s) to which they are bound.

In certain embodiments, a compound represented by:

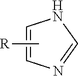

also includes where the R group replaces the H on the nitrogen atom.

Additionally, unless otherwise stated, structures depicted herein are also meant to include compounds that differ only in the presence of one or more isotopically enriched atoms. For example, compounds of this invention, wherein one or more hydrogen atoms are replaced deuterium or tritium, or one or more carbon atoms are replaced by a ¹³C— or ¹⁴C-enriched carbon are within the scope of this invention. Such compounds are useful, for example, as analytical tools, probes in biological assays, or antiviral compounds with improved therapeutic profile.

The terms “host” or “patient” mean human male or female, for example child, adolescent or adult.

It will be appreciated that the amount of a compound of the invention required for use in treatment will vary not only with the particular compound selected but also with the route of administration, the nature of the condition for which treatment is required and the age and condition of the patient and will be ultimately at the discretion of the attendant physician or veterinarian. In general however a suitable dose will be in the range of from about 0.1 to about 750 mg/kg of body weight per day, for example, in the range of 0.5 to 60 mg/kg/day, or, for example, in the range of 1 to 20 mg/kg/day.

The desired dose may conveniently be presented in a single dose or as divided dose administered at appropriate intervals, for example as two, three, four or more doses per day.

The compound is conveniently administered in unit dosage form; for example containing 10 to 1500 mg, conveniently 20 to 1000 mg, most conveniently 50 to 700 mg of active ingredient per unit dosage form.

Ideally the active ingredient should be administered to achieve peak plasma concentrations of the active compound of from about 1 to about 75 μM, about 2 to 50 μM, about 3 to about 30 μM. This may be achieved, for example, by the intravenous injection of a 0.1 to 5% solution of the active ingredient, optionally in saline, or orally administered as a bolus containing about 1 to about 500 mg of the active ingredient. Desirable blood levels may be maintained by a continuous infusion to provide about 0.01 to about 5.0 mg/kg/hour or by intermittent infusions containing about 0.4 to about 15 mg/kg of the active ingredient.

When the compounds of the present invention or a pharmaceutically acceptable salts thereof is used in combination with a second therapeutic agent active against the same virus the dose of each compound may be either the same as or differ from that when the compound is used alone. Appropriate doses will be readily appreciated by those skilled in the art.

While it is possible that, for use in therapy, a compound of the invention may be administered as the raw chemical it is preferable to present the active ingredient as a pharmaceutical composition. The invention thus further provides a pharmaceutical composition comprising compounds of the present invention or a pharmaceutically acceptable derivative thereof together with one or more pharmaceutically acceptable carriers therefore and, optionally, other therapeutic and/or prophylactic ingredients. The carrier(s) must be “acceptable” in the sense of being compatible with the other ingredients of the formulation and not deleterious to the recipient thereof.

Pharmaceutical compositions include those suitable for oral, rectal, nasal, topical (including buccal and sub-lingual), transdermal, vaginal or parenteral (including intramuscular, sub-cutaneous and intravenous) administration or in a form suitable for administration by inhalation or insufflation. The formulations may, where appropriate, be conveniently presented in discrete dosage units and may be prepared by any of the methods well known in the art of pharmacy. All methods include the step of bringing into association the active compound with liquid carriers or finely divided solid carriers or both and then, if necessary, shaping the product into the desired formulation.

Pharmaceutical compositions suitable for oral administration may conveniently be presented as discrete units such as capsules, cachets or tablets each containing a predetermined amount of the active ingredient; as a powder or granules; as a solution, a suspension or as an emulsion. The active ingredient may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The compounds according to the invention may also be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

For topical administration to the epidermis, the compounds according to the invention may be formulated as ointments, creams or lotions, or as a transdermal patch. Such transdermal patches may contain penetration enhancers such as linalool, carvacrol, thymol, citral, menthol and t-anethole. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or colouring agents.

Compositions suitable for topical administration in the mouth include lozenges comprising active ingredient in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Pharmaceutical compositions suitable for rectal administration wherein the carrier is a solid are for example presented as unit dose suppositories. Suitable carriers include cocoa butter and other materials commonly used in the art, and the suppositories may be conveniently formed by admixture of the active compound with the softened or melted carrier(s) followed by chilling and shaping in moulds.

Compositions suitable for vaginal administration may be presented as pessaries, tampons, creams, gels, pastes, foams or sprays containing in addition to the active ingredient such carriers as are known in the art to be appropriate.

For intra-nasal administration the compounds of the invention may be used as a liquid spray or dispersible powder or in the form of drops. Drops may be formulated with an aqueous or non-aqueous base also comprising one more dispersing agents, solubilizing agents or suspending agents. Liquid sprays are conveniently delivered from pressurized packs.

For administration by inhalation the compounds according to the invention are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount.

Alternatively, for administration by inhalation or insufflation, the compounds according to the invention may take the form of a dry powder composition, for example a powder mix of the compound and a suitable powder base such as lactose or starch. The powder composition may be presented in unit dosage form in, for example, capsules or cartridges or e.g. gelatin or blister packs from which the powder may be administered with the aid of an inhalator or insufflator.

When desired the above described formulations adapted to give sustained release of the active ingredient may be employed.

The following general schemes and examples are provided to illustrate various embodiments of the present invention and shall not be considered as limiting in scope. It will be appreciated by those of skill in the art that other compounds of the present invention can be obtained by substituting the generically or specifically described reactants and/or operating conditions used in the following examples.

In the foregoing and in the following examples, all temperatures are set forth uncorrected in degrees Celsius; and, unless otherwise indicated, all parts and percentages are by weight.

The following abbreviations may be used as follows:

| | |
|---|---|
| aq | aqueous |
| conc | concentrate |
| DCM | methylene chloride |
| DIPEA | Diisopropylethylamine |
| DMF | dimethylformamide |
| DMSO | Dimethylsulfoxide |
| EtOAc | Ethyl acetate |
| HATU | O-(7-Azabenzotriazol-1-yl)-N,N,N′,N′- |
| | tetramethyluronium hexafluorophosphate |
| M | molar |
| MeOH | Methanol |
| MTBE | methyl ter-butyl ether |
| n-BuLi | n-butyl lithium |
| PdCl₂dppf | (1,1′-Bis-(diphenylphosphino)- |
| | ferrocene)palladium (II) dichloride |
| Pd(PPh₃)₂Cl₂ | trans-dichlorobis(triphenyl phosphine) |
| | Palladium (II) |
| RT | room temperature |
| TEA | Triethylamine |
| THF | Tetrahydrofuran |

The compounds of this invention may be prepared in light of the specification using steps generally known to those of ordinary skill in the art. Those compounds may be analyzed by known methods, including but not limited to LCMS (liquid chromatography mass spectrometry) HPLC (high performance liquid chromatography) and NMR (nuclear magnetic resonance). It should be understood that the specific conditions shown below are only examples, and are not meant to limit the scope of the conditions that can be used for making compounds of this invention. Instead, this invention also includes conditions that would be apparent to those skilled in that art in light of this specification for making the compounds of this invention. Unless otherwise indicated, all variables in the following schemes are as defined herein. General Schemes:

Mass spec. samples were analyzed on a MicroMass Quattro Micro of MicroMass LCZ mass spectrometer operated in single MS mode with electrospray ionization. Samples were introduced into the mass spectrometer using chromatography. Mobile phase for all mass spec. analyses consisted of 10 mM pH 7 ammonium acetate and a 1:1 acetonitrile-methanol mixture. Method A: Column gradient conditions were 5%-100% acetonitrile-methanol over 3.5 mins gradient time and 4.8 mins run time on an ACE5C8 3.0×75 mm column. Flow rate was 1.2 ml/min. Method B: Column gradient were 5%-100% acetonitrile-methanol over 10 mins gradient time and 12 mins run time on a ACE5C8 4.6×150 mm column. Flow rate was 1.5 mL/min. As used herein, the term “Rt(min)” refers to the LCMS retention time, in minutes, associated with the compound. Unless otherwise indicated, the LCMS method utilized to obtain the reported retention time is as detailed above. If the Rt(min) is <5 min method A was used, if the Rt(min) is >5 min then method B was used.

1H-NMR spectra were recorded at 400 MHz using a Bruker DPX 400 or Varian instrument.

Purification by reverse phase HPLC is carried out under standard conditions using a Phenomenex Gemini C18 column, 21.2 mmID×250 mm, 5 μm, 110 Å. Elution is performed using a linear gradient 20 to 90% (CH₃CN in water or CH₃CN in water with 0.02% HCl) with a flow rate of 5.0 mL/minute.

### EXAMPLES

### Example 1

### Compound 7

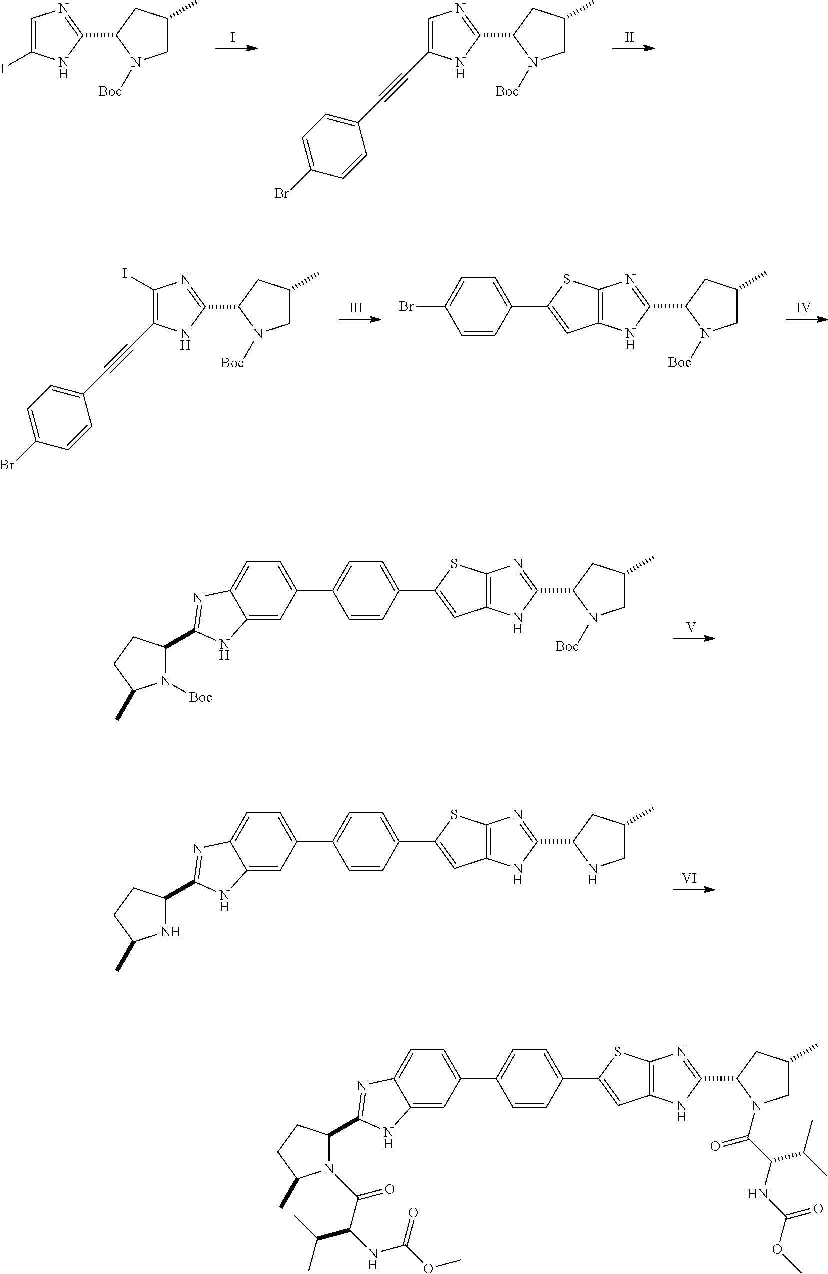

### Step I

### (2S,4S)-tert-butyl 2-(5-((4-bromophenyl)ethynyl)-1H-imidazol-2-yl)-4-methylpyrrolidine-1-carboxylate

A solution of 1-bromo-4-ethynyl-benzene (407.9 mg, 2.253 mmol), tert-butyl (2S,4S)-2-(5-iodo-1H-imidazol-2-yl)-4-methylpyrrolidine-1-carboxylate (850 mg, 2.253 mmol) and triethylamine (1.139 g, 11.26 mmol) in DMF (22 mL) was degassed by a N₂ flow for 15 min. To this solution was added Pd(DPPF)C₁₂-DCM (91.95 mg, 0.1126 mmol) and CuI (42.91 mg, 0.2253 mmol). The resulting solution was stirred at 80° C. for 16 h, cooled down to r.t. and diluted with EtOAc and water. The organic phase was extracted with EtoAc and dried over Na₂SO₄. After evaporation, the residue was purified by flash chromatography on silical gel (0 to 100% EtOAc in hexanes) to give (2S,4S)-tert-butyl 2-(5-((4-bromophenyl)ethynyl)-1H-imidazol-2-yl)-4-methylpyrrolidine-1-carboxylate (810 mg, 84%) as a brown solid.

¹H NMR (300.0 MHz, CDCl₃) d 7.47 (d, J=8.5 Hz, 2H), 7.38 (dd, J=2.0, 6.6 Hz, 2H), 7.27 (s, 1H), 4.93-4.87 (m, 1H), 3.79 (dd, J=7.4, 10.1 Hz, 1H), 2.87 (t, J=10.3 Hz, 1H), 2.62-2.45 (m, 2H), 2.28 (s, 2H), 1.51 (s, 9H) and 1.12 (d, J=6.4 Hz, 3H) ppm.

LC/MS: m/z=432.0 (M+H⁺), RT=3.40 min

### Step II

### (2S,4S)-tert-butyl 2-(5-((4-bromophenyl)ethynyl)-4-iodo-1H-imidazol-2-yl)-4-methylpyrrolidine-1-carboxylate

To a solution of tert-butyl (2S,4S)-2-[5-[2-(4-bromophenyl)ethynyl]-1H-imidazol-2-yl]-4-methyl-pyrrolidine-1-carboxylate (3.3 g, 7.668 mmol) in DCM (76.69 mL) was added NIS (1.898 g, 8.435 mmol) in one portion at 0° C. The resulting brownish solution was stirred at 0° C. while warming gradually to r.t. over 90 min. The solution was directly purified by flash chromatography on silica gel (0 to 100% EtOAc in hexanes) to give (2S,4S)-tert-butyl 2-(5-((4-bromophenyl)ethynyl)-4-iodo-1H-imidazol-2-yl)-4-methylpyrrolidine-1-carboxylate (4.1 g, 96%) as a yellow powder.

H NMR (300.0 MHz, CDCl3) d 11.02 (d, J=5.8 Hz, 1H), 7.51 (d, J=8.5 Hz, 2H), 7.41 (d, J=8.5 Hz, 2H), 4.89-4.83 (m, 1H), 3.81-3.72 (m, 1H), 2.87-2.81 (m, 1H), 2.48-2.44 (m, 2H), 2.24 (d, J=4.5 Hz, 1H), 1.51 (s, 9H) and 1.11 (d, J=6.5 Hz, 3H) ppm.

LC/MS: m/z=556.37 (M+H⁺), RT=3.81 min

### Step III

### (2S,4S)-tert-butyl 2-(5-(4-bromophenyl)-1H-thieno[2,3-d]imidazol-2-yl)-4-methylpyrrolidine-1-carboxylate

To a solution of tert-butyl (2S,4S)-2-[5-[2-(4-bromophenyl)ethynyl]-4-iodo-1H-imidazol-2-yl]-4-methyl-pyrrolidine-1-carboxylate (820 mg, 1.474 mmol) in DMF (16 mL) was added Na₂S.9H₂O (360.1 mg, 1.621 mmol) at r.t. The solution was then heated to 150° C. After 3 h, the solution is cooled to r.t. and dissolved with EtOAc and water, extracted with EtOAc and dried over Na₂SO₄. After evaporation, the residue was purified by flash chromatography on silical gel (0 to 100% EtOAc in hexanes) to give (2S,4S)-tert-butyl 2-(5-(4-bromophenyl)-1H-thieno[2,3-d]imidazol-2-yl)-4-methylpyrrolidine-1-carboxylate (190 mg, 28%) as a brown solid.

LC/MS: m/z=462.65 (M+H⁺), RT=3.69 min

### Step IV

### (2S,4S)-tert-butyl 2-(5-(4-(2-((2S,5S)-1-(tert-butoxycarbonyl)-5-methylpyrrolidin-2-yl)-1H-benzo[d]imidazol-6-yl)phenyl)-1H-thieno[2,3-d]imidazol-2-yl)-4-methylpyrrolidine-1-carboxylate

A solution of tert-butyl (2S,5S)-2-methyl-5-[6-(4,4,5,5-tetramethyl-1,3,2-dioxaborolan-2-yl)-1H-benzimidazol-2-yl]pyrrolidine-1-carboxylate (92.43 mg, 0.2163 mmol), (2S,4S)-tert-butyl 2-(5-(4-bromophenyl)-1H-thieno[2,3-d]imidazol-2-yl)-4-methylpyrrolidine-1-carboxylate (100 mg, 0.216 mmol), V-PHOS (16.98 mg, 0.03461 mmol) in isopropanol (1.745 mL)/NaHCO₃ (1.082 mL of 1 M, 1.082 mmol) was degassed for 15 min by a flow of N₂. Then Pd(OAc)₂ (1.94 mg, 0.0086 mmol) was added and the solution was heated to 100° C. for 6 h. The solution was cooled down to r.t. and diluted with EtOAc and water. The aqueous phase was extracted with EtOAc and the organic phase was dried over Na₂SO₄. After evaporation, the residue was purified by flash chromatography on silical gel (0 to 100% EtOAc in hexanes) to give (2S,4S)-tert-butyl 2-(5-(4-(2-((2S,5S)-1-(tert-butoxycarbonyl)-5-methylpyrrolidin-2-yl)-1H-benzo[d]imidazol-6-yl)phenyl)-1H-thieno[2,3-d]imidazol-2-yl)-4-methylpyrrolidine-1-carboxylate (66 mg, 40%) as a brown solid.

LC/MS: m/z=683.58 (M+H⁺), RT=3.41 min

### Step V

### 2-((2S,4S)-4-methylpyrrolidin-2-yl)-5-(4-(2-((2S,5S)-5-methylpyrrolidin-2-yl)-1H-benzo[d]imidazol-6-yl)phenyl)-1H-thieno[2,3-d]imidazole

A solution of (2S,4S)-tert-butyl 2-(5-(4-(2-((2S,5S)-1-(tert-butoxycarbonyl)-5-methylpyrrolidin-2-yl)-1H-benzo[d]imidazol-6-yl)phenyl)-1H-thieno[2,3-d]imidazol-2-yl)-4-methylpyrrolidine-1-carboxylate (66 mg, 0.09665 mmol) in HCl-dioxane (604.0 μL of 4 M, 2.416 mmol) was stirred for 1 h at r.t. The orange solution became gradually a suspension. After LCMS showed completion, the suspension was evaporated to dryness and a 100% yield (of the tetrahydrochloride salt) was assumed. Taken to the next step without further purification.

LC/MS: m/z=483.61 (M+H⁺), RT=0.52 min

### Step VI

### Compound 7

To a solution of (2S)-2-(methoxycarbonylamino)-3-methyl-butanoic acid (36.79 mg, 0.210 mmol), 2-[(2S,4S)-4-methylpyrrolidin-2-yl]-5-[4-[2-[(2S,5S)-5-methylpyrrolidin-2-yl]-1H-benzimidazol-5-yl]phenyl]-1H-thieno[2,3-d]imidazole (60 mg, 0.09547 mmol) and DIEA (123.4 mg, 166.3 μL, 0.954 mmol) in DCM (954 μL) was slowly (over 5 min) added t3p (182.3 μL of 50% w/v, 0.2864 mmol). The resulting solution was stirred at r.t. for 15 h after which time the solution was directly purified by flash chromatography on silica gel (0 to 20% MeOH in DCM) to give (Compound 7) (48 mg, 57%) as a pale yellow solid.

LC/MS: m/z=797.68 (M+H⁺), RT=2.90 min

### Example 2

### Synthesis of dimethyl (2S,2′S)-1,1′-((3S,3′S,5S,5′S)-5,5′-(5,5′-(buta-1,3-diyne-1,4-diyl)bis(1H-thieno[2,3-d]imidazole-5,2-diyl))bis(3-methylpyrrolidine-5,1-diyl))bis(3-methyl-1-oxobutane-2,1-diyl)dicarbamate (Compound 4)

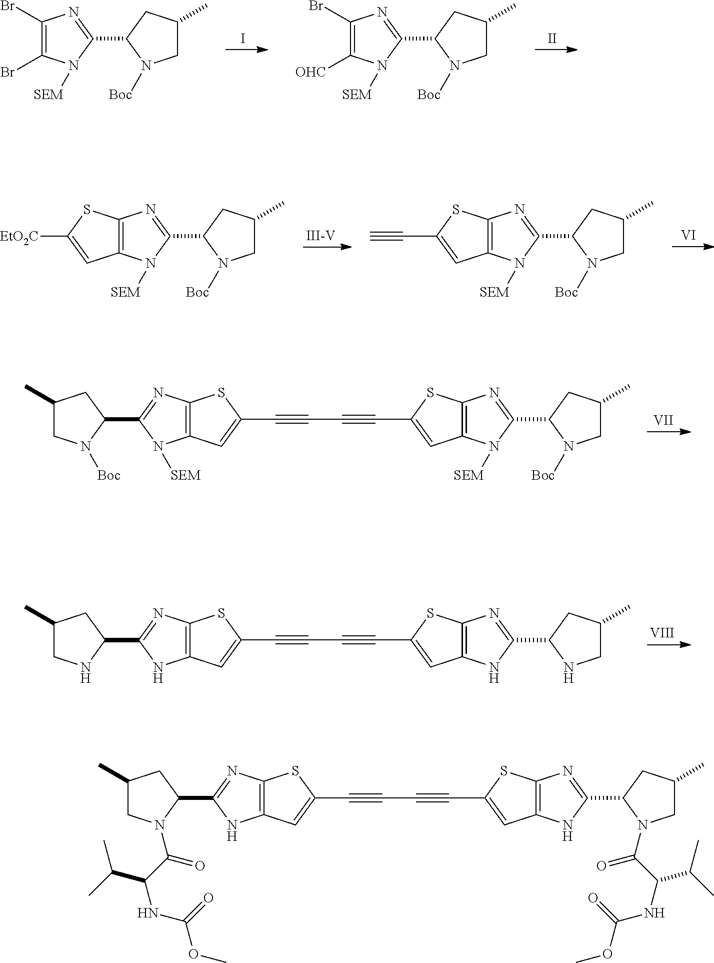

### Step I

### (2S,4S)-tert-butyl 2-(4-bromo-5-formyl-1-((2-(trimethylsilyl)ethoxy)methyl)-1H-imidazol-2-yl)-4-methylpyrrolidine-1-carboxylate

To a solution of tert-butyl (2S,4S)-2-[4,5-dibromo-1-(2-trimethylsilylethoxymethyl)imidazol-2-yl]-4-methyl-pyrrolidine-1-carboxylate (5.95 g, 11.03 mmol) in THF (110 mL) was added BuLi (5.0 mL of 2.5 M, 12.68 mmol) at −78 C. After 30 min, DMF (8.06 g, 8.54 mL, 110.3 mmol) was added to the solution and the reaction mixture was gradually warmed to r.t. overnight. The reaction mixture was then quenched with NH₄Cl, extracted with EtOAc. The organic phase was dried with Na₂SO₄. After evaporation, the residue was purified by flash chromatography on silica gel (0 to 100% EtOAc in hexanes) to afford the title compound (4.1 g, 75%) as a yellow oil.

LC/MS: m/z=488.33 (M+H⁺), RT=4.40 min

### Step II

### Ethyl 2-((2S,4S)-1-(tert-butoxycarbonyl)-4-methylpyrrolidin-2-yl)-1-((2-(trimethylsilyl)ethoxy)methyl)-1H-thieno[2,3-d]imidazole-5-carboxylate

A solution of tert-butyl (2S,4S)-2-[4-bromo-5-formyl-1-(2-trimethylsilylethoxymethyl)imidazol-2-yl]-4-methyl-pyrrolidine-1-carboxylate (4.1 g, 8.39 mmol), sodium ethanolate (3.42 g, 50.36 mmol) and ethyl 2-sulfanylacetate (2.01 g, 16.79 mmol) in EtOH (86.43 mL) is heated at 75° C. for 24 h. The solution was then cooled down to r.t. and directly purified by flash chromatography on silica gel (0 to 100% EtOAc in hexanes) to give the title compound (3.48 g, 81%) as an orange oil.

LC/MS: m/z=510.38 (M+H⁺), RT=4.39 min

### Step III-V

### (2S,4S)-tert-butyl 2-(5-ethynyl-1-((2-(trimethylsilyl)ethoxy)methyl)-1H-thieno[2,3-d]imidazol-2-yl)-4-methylpyrrolidine-1-carboxylate

### Step III

To a solution of ethyl 2-[(2S,4S)-1-tert-butoxycarbonyl-4-methyl-pyrrolidin-2-yl]-1-(2-trimethylsilylethoxymethyl)thieno[2,3-d]imidazole-5-carboxylate (2.47 g, 4.84 mmol) in DCM (50 mL) was added Dibal-H (14.5 mL of 1 M, 14.5 mmol) at −78° C. and was stirred at that temperature for 3 h. The reaction mixture was quenched with MeOH (1 mL) and was diluted with EtOAc. The resulting solution was stirred at r.t. for 30 min and the resulting cake was filtered through a Celite® pad. After evaporation, the residue was purified by flash chromatography on silica gel (0 to 100% EtOAc in hexanes) to afford the corresponding alcohol (1.67 g, 73%) as a colorless oil.

LC/MS: m/z=468.54 (M+H⁺), RT=3.58 min

### Step IV

To a solution of the above alcohol (835 mg, 1.78 mmol) in DCM (18 mL) was added Dess-Martin periodinane (795 mg, 1.87 mmol) at r.t. The solution was stirred at r.t. for 3 h and filtered through a pad of silica gel. The residue was taken directly onto step V.

LC/MS: m/z=466.66 (M+H⁺), RT=3.91 min

### Step V

To a solution of tert-butyl (2S,4S)-2-[5-formyl-1-(2-trimethylsilylethoxymethyl)thieno[2,3-d]imidazol-2-yl]-4-methyl-pyrrolidine-1-carboxylate (160 mg, 0.343 mmol) in MeOH (4.8 mL) was added K₂CO₃ (95.0 mg, 0.687 mmol) at rt. The resulting yellow solution was stirred at r.t. for 16 h. After evaporation, the residue was purified by flash chromatography on silica gel (0 to 100% EtOAc in hexanes) to afford (2S,4S)-tert-butyl 2-(5-ethynyl-1-((2-(trimethylsilyl)ethoxy)methyl)-1H-thieno[2,3-d]imidazol-2-yl)-4-methylpyrrolidine-1-carboxylate (0.1 g, 63%) as a colorless oil.

LC/MS: m/z=462.42 (M+H⁺).

### Step VI

### (3S,3′S,5S,5′S)-tert-butyl 5,5′-(5,5′-(buta-1,3-diyne-1,4-diyl)bis(1-((2-(trimethylsilyl)ethoxy)methyl)-1H-thieno[2,3-d]imidazole-5,2-diyl))bis(3-methylpyrrolidine-1-carboxylate)

A non-degassed solution of tert-butyl (2S,4S)-2-[5-ethynyl-1-(2-trimethylsilylethoxymethyl)thieno[2,3-d]imidazol-2-yl]-4-methyl-pyrrolidine-1-carboxylate (88 mg, 0.19 mmol), CuI (3.6 mg, 0.019 mmol), PdCl₂(PPh₃)₂ (6.6 mg, 0.0095 mmol), TEA (48.2 mg, 66.4 μL, 0.47 mmol) and 1,4-benzoquinone (20.6 mg, 0.190 mmol) in THF (2 mL) is stirred at r.t. for 3 h. The solution was then filtered through a pad of Celite and was purified by flash chromatography on silica gel (0 to 100% EtOAc in hexanes) to afford the diyne product (87 mg, 99%) as a brown solid.

LC/MS: m/z=921.99 (M+H⁺), RT=3.97 min

### Step VII

### 1,4-bis(2-((2S,4S)-4-methylpyrrolidin-2-yl)-1H-thieno[2,3-d]imidazol-5-yl)buta-1,3-diyne

A solution of tert-butyl (2S,4S)-2-[5-[4-[2-[(2S,4S)-1-tert-butoxycarbonyl-4-methyl-pyrrolidin-2-yl]-1-(2-trimethylsilylethoxymethyl)thieno[2,3-d]imidazol-5-yl]buta-1,3-diynyl]-1-(2-trimethylsilylethoxymethyl)thieno[2,3-d]imidazol-2-yl]-4-methyl-pyrrolidine-1-carboxyla (87 mg, 0.094 mmol) in HCl-dioxane (1.259 mL of 4 M, 5.035 mmol) was stirred for 2 h. The a few drop of MeOH were added and the solution was stirred overnight to get the 2 SEM group off. The solution was evaporated to dryness to give a 1,4-bis(2-((2S,4S)-4-methylpyrrolidin-2-yl)-1H-thieno[2,3-d]imidazol-5-yl)buta-1,3-diyne 4HCl as a white powder that was directly taken to the next step.

LC/MS: m/z=461.42 (M+H⁺), RT=1.38 min

### Step VIII

### dimethyl (2S,2′S)-1,1′-((3S,3′S,5S,5′S)-5,5′-(5,5′-(buta-1,3-diyne-1,4-diyl)bis(1H-thieno[2,3-d]imidazole-5,2-diyl))bis(3-methylpyrrolidine-5,1-diyl))bis(3-methyl-1-oxobutane-2,1-diyl)dicarbamate

To a solution of 2-[(2S,4S)-4-methylpyrrolidin-2-yl]-5-[4-[2-[(2S,4S)-4-methylpyrrolidin-2-yl]-1H-thieno[2,3-d]imidazol-5-yl]buta-1,3-diynyl]-1H-thieno[2,3-d]imidazole (21 mg, 0.045 mmol), (2S)-2-(methoxycarbonylamino)-3-methyl-butanoic acid (17.5 mg, 0.100 mmol) and DIEA (58.9 mg, 79.4 μL, 0.455 mmol) in DCM (363 μL) was slowly added t3p (87 μL of 50% w/v, 0.136 mmol) at r.t. The resulting solution was stirred at r.t. for 5 h. The reaction mixture was directly purified by flash chromatography on silica gel (0 to 20% MeOH in DCM) to afford dimethyl (2S,2′S)-1,1′-((3S,3′S,5S,5′S)-5,5′-(5,5′-(buta-1,3-diyne-1,4-diyl)bis(1H-thieno[2,3-d]imidazole-5,2-diyMbis(3-methylpyrrolidine-5,1-diyl))bis(3-methyl-1-oxobutane-2,1-diyl)dicarbamate (20 mg, 55%) as a dark orange crystals.

LC/MS: m/z=775.48 (M+H⁺), RT=3.21 min

### Example 3

### Compound II

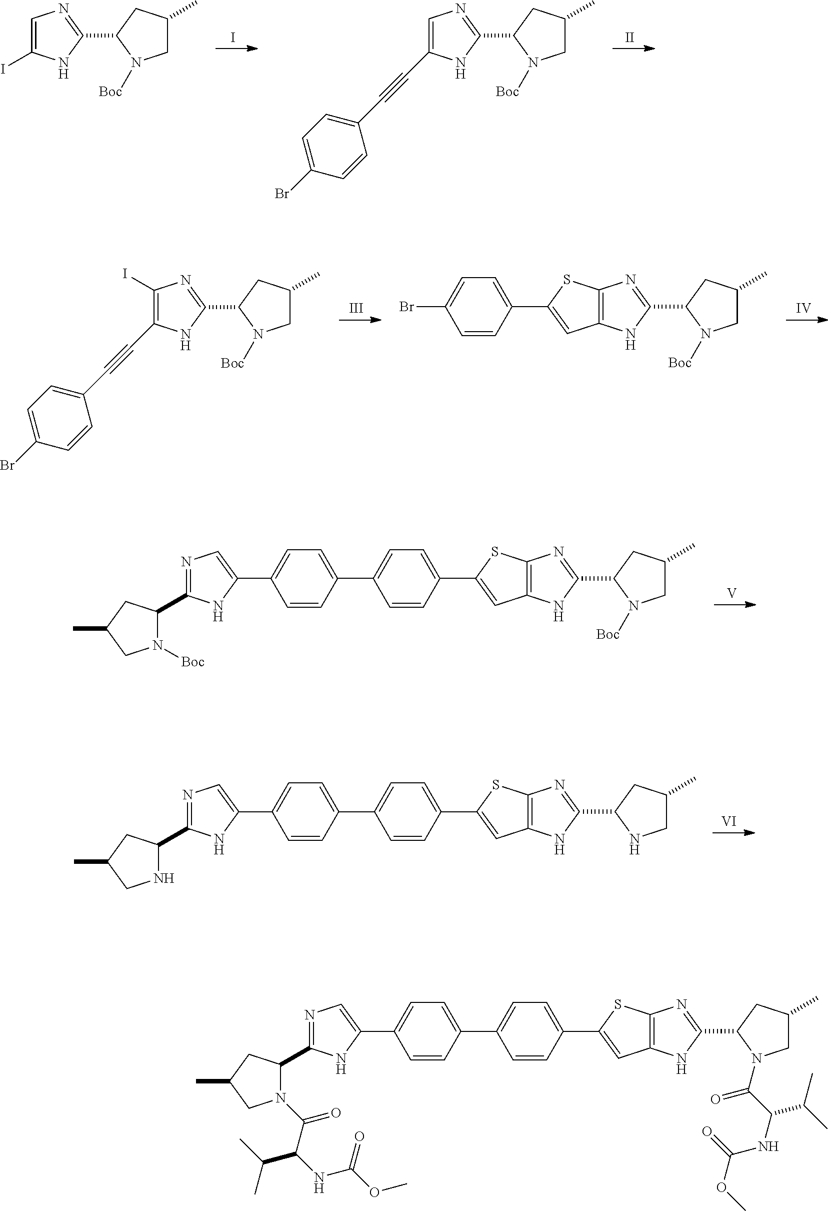

### Step I

### (2S,4S)-tert-butyl 2-(5-((4-bromophenyl)ethynyl)-1H-imidazol-2-yl)-4-methylpyrrolidine-1-carboxylate

A solution of 1-bromo-4-ethynyl-benzene (407.9 mg, 2.253 mmol), tert-butyl (2S,4S)-2-(5-iodo-1H-imidazol-2-yl)-4-methylpyrrolidine-1-carboxylate (850 mg, 2.253 mmol) and triethylamine (1.139 g, 11.26 mmol) in DMF (22 mL) was degassed by a N₂ flow for 15 min. To this solution was added Pd(DPPF)C₁₂-DCM (91.95 mg, 0.1126 mmol) and CuI (42.91 mg, 0.2253 mmol). The resulting solution was stirred at 80° C. for 16 h, cooled down to r.t. and diluted with EtOAc and water. The organic phase was extracted with EtoAc and dried over Na₂SO₄. After evaporation, the residue was purified by flash chromatography on silical gel (0 to 100% EtOAc in hexanes) to give (2S,4S)-tert-butyl 2-(5-((4-bromophenyl)ethynyl)-1H-imidazol-2-yl)-4-methylpyrrolidine-1-carboxylate (810 mg, 84%) as a brown solid.

¹H NMR (300.0 MHz, CDCl₃) δ 7.47 (d, J=8.5 Hz, 2H), 7.38 (dd, J=2.0, 6.6 Hz, 2H), 7.27 (s, 1H), 4.93-4.87 (m, 1H), 3.79 (dd, J=7.4, 10.1 Hz, 1H), 2.87 (t, J=10.3 Hz, 1H), 2.62-2.45 (m, 2H), 2.28 (s, 2H), 1.51 (s, 9H) and 1.12 (d, J=6.4 Hz, 3H) ppm.

LC/MS: m/z=432.0 (M+H⁺), RT=3.40 min

### Step II

### (2S,4S)-tert-butyl 2-(5-((4-bromophenyl)ethynyl)-4-iodo-1H-imidazol-2-yl)-4-methylpyrrolidine-1-carboxylate

To a solution of tert-butyl (2S,4S)-2-[5-[2-(4-bromophenyl)ethynyl]-1H-imidazol-2-yl]-4-methyl-pyrrolidine-1-carboxylate (3.3 g, 7.668 mmol) in DCM (76.69 mL) was added NIS (1.898 g, 8.435 mmol) in one portion at 0° C. The resulting brownish solution was stirred at 0° C. while warming gradually to r.t. over 90 min. The solution was directly purified by flash chromatography on silica gel (0 to 100% EtOAc in hexanes) to give (2S,4S)-tert-butyl 2-(5-((4-bromophenyl)ethynyl)-4-iodo-1H-imidazol-2-yl)-4-methylpyrrolidine-1-carboxylate (4.1 g, 96%) as a yellow powder.

¹H NMR (300.0 MHz, CDCl₃) δ 11.02 (d, J=5.8 Hz, 1H), 7.51 (d, J=8.5 Hz, 2H), 7.41 (d, J=8.5 Hz, 2H), 4.89-4.83 (m, 1H), 3.81-3.72 (m, 1H), 2.87-2.81 (m, 1H), 2.48-2.44 (m, 2H), 2.24 (d, J=4.5 Hz, 1H), 1.51 (s, 9H) and 1.11 (d, J=6.5 Hz, 3H) ppm.

LC/MS: m/z=556.37 (M+H⁺), RT=3.81 min

### Step III

### (2S,4S)-tert-butyl 2-(5-(4-bromophenyl)-1H-thieno[2,3-d]imidazol-2-yl)-4-methylpyrrolidine-1-carboxylate

To a solution of tert-butyl (2S,4S)-2-[5-[2-(4-bromophenyl)ethynyl]-4-iodo-1H-imidazol-2-yl]-4-methyl-pyrrolidine-1-carboxylate (820 mg, 1.474 mmol) in DMF (16 mL) was added Na₂S.9H₂O (360.1 mg, 1.621 mmol) at r.t. The solution was then heated to 150° C. After 3 h, the solution is cooled to r.t. and dissolved with EtOAc and water, extracted with EtOAc and dried over Na₂SO₄. After evaporation, the residue was purified by flash chromatography on silica gel (0 to 100% EtOAc in hexanes) to give (2S,4S)-tert-butyl 2-(5-(4-bromophenyl)-1H-thieno[2,3-d]imidazol-2-yl)-4-methylpyrrolidine-1-carboxylate (190 mg, 28%) as a brown solid.

LC/MS: m/z=462.65 (M+H⁺), RT=3.69 min

### Step IV

### (2S,4S)-tert-butyl 2-(5-(4′-(2-((2S,4S)-1-(tert-butoxycarbonyl)-4-methylpyrrolidin-2-yl)-1H-imidazol-5-yl)biphenyl-4-yl)-1H-thieno[2,3-d]imidazol-2-yl)-4-methylpyrrolidine-1-carboxylate

A solution of (2S,4S)-tert-butyl 4-methyl-2-(4-(4-(4,4,5,5-tetramethyl-1,3,2-dioxaborolan-2-yl)phenyl)-1H-imidazol-2-yl)pyrrolidine-1-carboxylate (981 mg, 2.16 mmol), 2S,4S)-tert-butyl 2-(5-(4-bromophenyl)-1H-thieno[2,3-d]imidazol-2-yl)-4-methylpyrrolidine-1-carboxylate (1 g, 2.16 mmol), V-PHOS (333 mg, 0.65 mmol) in isopropanol (21.6 mL)/NaHCO₃ (10.8 mL of 1 M, 10.8 mmol) was degassed for 30 min by a flow of N₂. Then Pd(OAc)₂ (39 mg, 0.17 mmol) was added and the solution was heated to 100° C. for 15 h. The solution was cooled down to r.t. and diluted with EtOAc and water. The aqueous phase was extracted with EtOAc and the organic phase was dried over Na₂SO₄. After evaporation, the residue was purified by flash chromatography on silica gel (0 to 100% EtOAc in hexanes) to give (2S,4S)-tert-butyl 2-(5-(4′-(2-((2S,4S)-1-(tert-butoxycarbonyl)-4-methylpyrrolidin-2-yl)-1H-imidazol-5-yl)biphenyl-4-yl)-1H-thieno[2,3-d]imidazol-2-yl)-4-methylpyrrolidine-1-carboxylate (880 mg, 57%) as a yellow solid.

LC/MS: m/z=709.31 (M+H⁺), RT=2.15 min

### Steps V and VI

### Compound II

A solution of (2S,4S)-tert-butyl 2-(5-(4′-(2-((2S,4S)-1-(tert-butoxycarbonyl)-4-methylpyrrolidin-2-yl)-1H-imidazol-5-yl)biphenyl-4-yl)-1H-thieno[2,3-d]imidazol-2-yl)-4-methylpyrrolidine-1-carboxylate (58 mg, 0.09665 mmol) in TFA (1 mL) and DCM (1 mL) was stirred for 1 h at r.t. The solvent were removed in vacuo to afford (2-((2S,4S)-4-methylpyrrolidin-2-yl)-5-(4′-(2-((2S,4S)-4-methylpyrrolidin-2-yl)-1H-imidazol-5-yl)biphenyl-4-yl)-1H-thieno[2,3-d]imidazole.3TFA which was taken directly to the next step.

LC/MS: m/z=509.73 (M+H⁺), RT=1.53 min

To a solution of (S)-2-(methoxycarbonylamino)-3-methylbutanoic acid (30.1 mg, 0.17 mmol), 2-((2S,4S)-4-methylpyrrolidin-2-yl)-5-(4′-(2-((2S,4S)-4-methylpyrrolidin-2-yl)-1H-imidazol-5-yl)biphenyl-4-yl)-1H-thieno[2,3-d]imidazole 3TFA (69.6 mg, 0.081 mmol), HATU (65.3, 0.17 mmol) in DMF (2 mL) was added DIEA (142.5 uL, 0.81 mmol). The solution was then stirred at r.t. for 3 h. The reaction was then diluted with H₂O and EtOAc. The aqueous phase was extracted with EtOAc. The combined organic phase were dried using MgSO₄, evaporated and the residue was purified by flash chromatography to afford Compound 11 (49.7 mg, 73%) as a white powder.

LC/MS: m/z=823.09 (M+H⁺), RT=2.09 min

Compounds I-3,5,6,8-10, 12-32, 1a-12a, 1b-4-b, 1c, and 2c

Compounds I-3,5,6,8-10, 12-32 as disclosed in Tables 1A, compounds 1a-12a as dosclosed in Table 1B, compounds 1b-4-b as disclosed in Table 3, and compounds Ic and 2c as disclosed in Table 4 were prepared according to the procedures outlined in Examples 1-3 using the appropriate intermediate starting materials.

### Example 4

### Activity Determination Using the ELISA and the Sub-Genomic Replicon 1a Cell Line

The cell line W11.8 containing the sub-genomic HCV replicon of genotype 1a is used to determine the potency of the drugs. The RNA replication in presence of different drug concentrations is indirectly measured in this cell line by the level of NS5A protein content upon drug treatment for four days. It is shown that the level of the NS5A protein correlates well with the level of HCV RNA in the replicon cell line. Cells are split twice a week in order to keep the confluence state below 85% of the culture flask surface area. The culture media used for cell passaging consists of DMEM-10% foetal bovine serum with 100 UI/mL penicillin, 100 μg/mL streptomycin, 2 mM glutamine, 1 mM sodium pyruvate, non-essential amino acids (1×) and 600 μg/mL of G418 final concentrations. Monolayer of the W11.8 cells is trypsinized and cells are counted. Cells are diluted at 50,000 cells/mL with complete DMEM without G418, then approximately 5,000 viable cells (100 μL) are plated per well in a white opaque 96-well microtiter plate. After an incubation period of 2-4 hours at 37° C. in a 5% CO2 incubator, compounds are added at various concentrations. Drugs are resuspended in DMSO at a stock concentration of 10 mM. Then, drugs are serially diluted at twice the final concentration in the same medium. One volume (100 μL) of each drug dilution is then added to each well that contains cells. A control compound is used as an internal standard for each plate assay. Sixteen wells are used as control (0% inhibition) without drug. Eight wells are used as background control (100% inhibition) containing 2 μM (final concentration) of the control drug that was shown to inhibit the NS5A expression at ≈100% and is nontoxic to the cells. Values from 100% inhibited wells were averaged and used as the background value. Cells are further incubated for four days at 37° C. in a 5% CO₂ incubator. Following the incubation time of four days, the media is removed and wells are washed once with 150 μL of PBS at room temperature for five minutes. Cells are then fixed for five minutes using 150 μL per well of cold (−20° C.) fixative solution (50% methanol/50% acetone mix). Cells are then washed twice with 150 μL of PBS (phosphate buffered saline) per well, following the addition of 150 μL of blocking solution, cells are incubated for one hour at 37° C. to block non-specific sites. The blocking solution is removed and cells are washed twice with 150 μL of PBS per well and once with 150 μL of PBSTS solution (PBS/0.1% Triton X-100/0.02% SDS) per well. Then, 50 μL of mouse monoclonal anti-NS5A antibody (Santa Cruz, Cat. No. sc-52417) is added in each well, diluted 1/1,000 in the blocking solution and incubated at 4° C. overnight. Next day, media is removed and plates are washed five times with 150 μL of PBS per well with five-minute incubations at room temperature. Then 50 μL per well of peroxidase-conjugated donkey anti-mouse antibody (Jackson Immunoresearch, Cat. No. 715-036-150) diluted 1/10,000 in the blocking solution is added and incubated at room temperature for three hours on a shaker (500 rpm). Plates are washed four times with 150 μL of PBSTS solution per well and once with 150 μL of PBS. Then, substrate solution (100 μl, SuperSignal ELISA Pico Chemiluminescent Substrate, Fisher Cat. No. 37069) is added in each well and plates are incubated 60 minutes at room temperature prior to reading the luminescence (relative light units) on the Analyst HT plate reader. The percentage of inhibition at each drug concentration tested (in duplicate) is calculated. The concentration required to reduce viral replication by 50% (IC₅₀) is then determined from dose response curves using nonlinear regression analysis with the GraphPad Prism software, version 2.0 (GraphPad Software Inc., San Diego, Calif., USA).

### Example 5

### Cell-Based Luciferase Reporter HCV (Ib) RNA Replication Assay Cell Culture

Replicon cell lines Huh-5.2 are derived from the Huh-7 hepatocarcinoma cell line are maintained in culture as generally described in Krieger, N; Lohmann, V; Bartenschlager, R. Enhancement of hepatitis C virus RNA replication by cell culture-adaptive mutations. J. Virol. 2001, 75, 4614-4624. The Huh-5.2 cells contain the highly cell culture-adapted replicon I₃₈₉Luc-ubi-neo/NS3-3′/5.1 construct that carries, in addition to the neomycin gene, an integrated copy to the firefly luciferase gene (Krieger, N; Lohmann, V; Bartenschlager, R. Enhancement of hepatitis C virus RNA replication by cell culture-adaptive mutations. J. Virol. 2001, 75, 4614-4624). This cell line allows measurement of HCV RNA replication and translation by measuring luciferase activity. It has been previously shown that the luciferase activity tightly follows the replicon RNA level in these cells (Krieger, N; Lohmann, V; Bartenschlager, R. Enhancement of hepatitis C virus RNA replication by cell culture-adaptive mutations. J. Virol. 2001, 75, 4614-4624). The Huh-ET cell line has the same features as those mentioned for Huh-5.2 cell line, except that ET cells are more robust and contain an adaptative mutation in the HCV NS4B gene instead of NSSA. Both cell lines are maintained in cultures at a sub-confluent level (<85%) as the level of replicon RNA is highest in actively proliferating cells. The culture media used for cell passaging consist of DMEM (Gibco BRL Laboratories, Mississauga, ON, Canada) supplemented with 10% foetal bovine serum with 1% penicilin/streptomycin, 1% glutamine, 1% sodium pyruvate, 1% non-essential amino acids, and 180 μg/ml of G418 final concentration. Cells are incubated at 37° C., in an atmosphere of 5% CO₂ and passaged twice a week to maintain sub-confluence.

Approximately 3000 viable Huh-ET cells (100 μl) are plated per well in a white opaque 96-well microtiter plate. The cell culture media used for the assay is the same as described above except that it contains no G418 and no phenol red. After an incubation period of 3-4 hours at 37° C. in a 5% CO₂ incubator, compounds (100 μl) are added at various concentrations. Cells are then further incubated for 4 days at 37° C. in a 5% CO₂ incubator. Thereafter, the culture media is removed and cells are lysed by the addition of 95 μL of the luciferase buffer (luciferin substrate in buffered detergent). Cell lysates are incubated at room temperature and protected from direct light for at least 10 minutes. Plates are read for luciferase counts using a luminometer (Wallac MicroBeta Trilux, Perkin Elmer™, MA, USA).

HCV 1a and 1b are the two most prevalent HCV genotypes and the most difficult to treat. It has proven problematic in the past to find compounds having good activity against both genotypes. However, the compounds of the present invention, particularly those with a 4-methylpyrrolidine group, are active against both HCV 1a and 1b genotypes. The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

The 50% inhibitory concentrations (IC₅₀s) for inhibitory effect are determined from dose response curves using eleven concentrations per compound in duplicate. Curves are fitted to data points using nonlinear regression analysis, and IC₅₀s are interpolated from the resulting curve using GraphPad Prism software, version 2.0 (GraphPad Software Inc., San Diego, Calif., USA).

Tables 2A and 2B show compounds representative of the present invention

TABLE 2A

| | M + 1 | RT | | EC50 |
|---|---|---|---|---|
| # | (obs) | (min) | 1-H -NMR | 1b |
| 1 | 717.68 | 3.72 | | +++ |
| 2 | 747.45 | 4.04 | | +++ |
| 3 | 745.66 | 3.46 | | +++ |
| 4 | 775.48 | 3.22 | | +++ |
| 5 | 803.68 | 3.56 | | +++ |
| 6 | 683.58 | 3.39 | | |
| 7 | 797.68 | 2.9 | | +++ |
| 8 | 798.52 | 3.33 | “H NMR (300.0 MHz, Acetone) d 7.91-7.46 (m, 9H), 6.43 | +++ |
| | | | (d, J = 8.7 Hz, 1H), 6.16 (s, 1H), 5.23 (s, 1H), 4.85 (s, 1H), | |
| | | | 4.27-4.15 (m, 4H), 3.77-3.69 (m, 1H), 3.61 (s, 6H), 3.25 (d, | |
| | | | J = 3.7 Hz, 2H), 2.85 (s, 3H), 2.50-2.17 (m, 3H), 1.55 (d, J = | |
| | | | 6.4 Hz, 3H), 1.41 (m, 12H), 0.98 (d, J = 5.9 Hz, 3H) and 0.85- | |
| | | | 0.80 (m, 1H) ppm” | |
| 9 | 811.18 | 3 | “H NMR (300.0 MHz, Acetone) d 11.48 (s, 1H), 7.74-7.33 | +++ |
| | | | (m, 8H), 6.23 (d, J = 8.4 Hz, 1H), 6.09 (d, J = 8.5 Hz, 1H), | |
| | | | 5.33-5.28 (m, 1H), 5.23-5.18 (m, 1H), 4.31-4.25 (m, 2H), | |
| | | | 3.99 (s, 3 H), 3.59 (s, 6 H), 3.41 (s, 1 H), 3.23 (s, 1 H), 2.99 | |
| | | | (s, 1 H), 2.64-2.32 (m, 4H), 1.98 (s, 2H), 1.40 (d, J = 5.3 Hz, | |
| | | | 3H) and 1.06-0.56 (m, 18H) ppm” | |
| 10 | 796.56 | 3.59 | “H NMR (300.0 MHz, Acetone) d 11.11 (s, 2H), 10.22 (m, | +++ |
| | | | 1H), 7.68-7.46 (m, 8H), 7.46-7.32 (m, 1H), 6.44-6.32 (m, | |
| | | | 1H), 6.15 (d, J = 7.1 Hz, 1H), 4.34-4.21 (m, 2H), 3.78-3.68 | |
| | | | (m, 4H), 3.60 (s, 3H), 3.22 (s, 3H), 2.61 (s, 1H), 2.36 (s, 1H) | |
| | | | and 1.42-0.80 (m, 25H) ppm” | |
| 11 | 823.09 | 2.09 | ¹H NMR (300 MHz, DMSO) δ 12.30 (m, 2H), 7.97-7.62 (m, | +++ |
| | | | 8H), 7.51 (dd, J= 7.9, 3.8 Hz, 2H), 7.20 (m, 2H), 4.96 (dd, J = | |
| | | | 18.7, 9.2 Hz, 2H), 4.18-3.95 (m, 4H), 3.54 (s, 6H), 3.23 | |
| | | | (m, 2H), 2.46-2.09 (m, 4H), 1.95-1.63 (m, 4H), 1.10 (d, J = | |
| | | | 6.3 Hz, 6H), 0.82 (dt, J = 19.3, 6.9 Hz, 12H). | |
| 12 | 771.51 | 2.23 | | +++ |
| 13 | 797.15 | 2.82 | | +++ |
| 14 | 821.53 | 3.07 | “1H NMR (300 MHz, DMSO) d 7.93 (s, 1H), 7.75 (dd, J = | +++ |
| | | | 8.2, 6.0 Hz, 3H), 7.67-7.49 (m, 4H), 7.27-7.08 (m, 2H), | |
| | | | 5.20-5.08 (m, 1H), 5.01 (t, J = 8.6 Hz, 1H), 4.27-3.97 (m, | |
| | | | 4H), 3.54 (s, 6H), 3.38 (d, J = 9.9 Hz, 1H), 2.65-2.18 (m, H), | |
| | | | 2.01-1.60 (m, 4H), 1.12 (dd, J = 8.1, 6.5 Hz, 6H), 0.80 | |
| | | | (m, 12H).” | |
| 15 | 847.79 | 2.55 | | +++ |
| 16 | 885.72 | 3.26 | | +++ |
| 17 | 823.8 | 2.25 | | +++ |
| 18 | 765.78 | 3.66 | “1H NMR (300 MHz, DMSO) d 7.97 (s, 1H), 7.87-7.72 (m, | ++ |
| | | | 6H), 7.67 (d, J = 3.8 Hz, 1H), 7.64 (d, J = 3.9 Hz, 1H), 7.60 | |
| | | | (br. s, 1H), 5.15-5.04 (m, 2H), 4.83 (m, 2H), 3.77 (m, 3H), | |
| | | | 3.22-2.94 (m, 3H), 2.75-2.27 (m, H), 1.75 (m, 3H), 1.39 (s, | |
| | | | 6H), 1.22-0.84 (m, 18H).” | |
| 19 | 797.76 | 2.14 | | +++ |
| 20 | 823.73 | 2.06 | | +++ |
| 21 | 853.5 | 3.13 | | +++ |
| 22 | 829.14 | 2.95 | | +++ |
| 23 | 879.66 | 2.23 | | ++ |
| 24 | 879.81 | 3.1 | | +++ |
| 25 | 829.73 | 2.14 | | +++ |
| 26 | 885.71 | 2.07 | | +++ |
| 27 | 859.93 | 2.21 | | +++ |
| 28 | 837.46 | 2.07 | | +++ |
| 29 | 885.47 | 2.4 | | +++ |
| 30 | 885.63 | 3.23 | “1H NMR (300 MHz, DMSO) d 7.78-7.68 (m, 4H), 7.65 (s, | +++ |
| | | | 1H), 7.56 (d, J = 3.8 Hz, 1H), 7.40 (s, 1H), 7.36 (d, J = 3.8 | |
| | | | Hz, 1H), 7.26 (2 br s, 2H), 5.11-4.92 (m, 4H), 4.19 (t, J = 8.2 | |
| | | | Hz, 2H), 4.05 (t, J = 7.1 Hz, 2H), 3.53 (s, 6H), 3.40-3.15 (m, | |
| | | | 2H), 2.30 (m, 2H), 2.00-1.66 (m, 4H), 1.20-1.05 (m, 6H), | |
| | | | 0.78 (t, J = 7.0 Hz, 12H).” | |
| 31 | 777.57 | 2.3 | “1H NMR (300 MHz, DMSO) d 7.93 (s, 1H), 7.44 (d, J = 3.9 | +++ |
| | | | Hz, 1H), 7.42 (s, 1H), 7.32 (d, J = 3.8 Hz, 1H), 7.30 (d, J = | |
| | | | 7.7 Hz, 1H), 7.24 (d, J = 8.2 Hz, 1H), 4.96 (dd, J = 10.3, 6.6 | |
| | | | Hz, 4H), 4.28-4.12 (m, 2H), 4.04 (m, 2H), 3.53 (s, 6H), 3.26 | |
| | | | (m, 2H), 2.44-2.17 (m, 4H), 1.96-1.60 (m, 4H), 1.10 (d, J = | |
| | | | 6.3 Hz, 6H), 0.89-0.63 (m, 12H).” | |
| 32 | 873.34 | 2.99 | | +++ |

TABLE 2B

| | M + 1 | | EC50 |
|---|---|---|---|
| # | (obs) | 1-H -NMR | 1b |
| 1a | 857.42 | H NMR (400.0 MHz, Acetone) d 11.96 (s, 1H), 11.61 (s, 1H), | +++ |
| | | 7.78-7.70 (m, 8H), 7.36 (s, 1H), 6.31-6.24 (m, 2H), 5.25- | |
| | | 5.21 (m, 1H), 5.11-5.07 (m, 1H), 4.30-4.20 (m, 4H), 4.05 | |
| | | (q, J = 7.1 Hz, 1H), 3.60 (s, 6H), 3.25 (s, 2 H), 2.10-2.00 (m, | |
| | | 7H), 1.22-1.12 (m, 6H) and 0.93-0.82 (m, 12H) ppm [1] | |
| 2a | 891.36 | H NMR (400.0 MHz, Acetone) d 12.18 (s, 1H), 7.82-7.68 | +++ |
| | | (m, 8H), 6.33 (t, J = 7.9 Hz, 1 H), 5.25-5.21 (m, 1H), 5.13- | |
| | | 5.09 (m, 1H), 4.32-4.22 (m, 2H), 4.05 (q, J = 7.1 Hz, 2H), | |
| | | 3.41-3.27 (m, 2H), 2.58-2.32 (m, 4H), 2.05 (m, 12H), 1.22- | |
| | | 1.12 (m, 6H) and 0.92-0.76 (m, 12H) ppm [1] | |
| 3a | 949.82 | H NMR (400.0 MHz, Acetone) d 11.68 (s, 1 H), 11.49 (s, | +++ |
| | | 1H), 7.93-7.70 (m, 8H), 7.40 (s, 1 H), 6.21 (s, 2H), 5.23 (m, | |
| | | 1H), 5.11 (m, 1H), 4.37-4.21 (m, 4H), 4.08-3.99 (m, 1H), | |
| | | 3.60 (s, 6H), 3.29-3.22 (m, 2H), 2.05-1.97 (m, 7H), 1.22- | |
| | | 1.14 (m, 6H) and 0.92-0.74 (m, 12H) ppm [1] | |
| 4a | 727.51 | | +++ |
| 5a | 837.73 | | +++ |
| 6a | 841.68 | | +++ |
| 7a | 841.6 | | +++ |
| 8a | 837.68 | | +++ |
| 9a | 841.52 | | +++ |
| 10a | | | +++ |
| 12a | | | +++ |
| 13a | 837.63 | | +++ |

uM: +++ <= 0.005 < ++ <= 5.0 < +

Table 3 shows comparative data for exemplary compounds of formula (I), some of which have a substituent at the 4-position of the pyrrolidine ring (i.e., compounds of the invention where R₄ and R_{4′} are methyl). Data shows EC₅₀ values against the sub-genomic replicon 1a and 1b cell lines. According to an aspect of the invention, the compounds of the invention are selected from Table 3 or a pharmaceutically acceptable salt thereof.

TABLE 3

| | | EC₅₀ (μM) |
|---|---|---|
| Entry | Structure | (1a) |
| 1b | 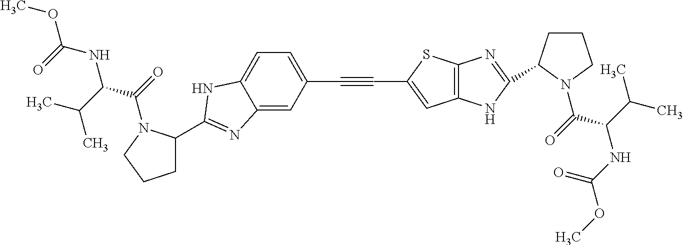 [SMILES: COC(=O)N[C@H](C(=O)N1CCCC1c1nc2cc(C#Cc3cc4[nH]c([C@@H]5CCCN5C(=O)[C@@H](NC(=O)OC)C(C)C)nc4s3)ccc2[nH]1)C(C)C] | 0.8281 |
| 2b | 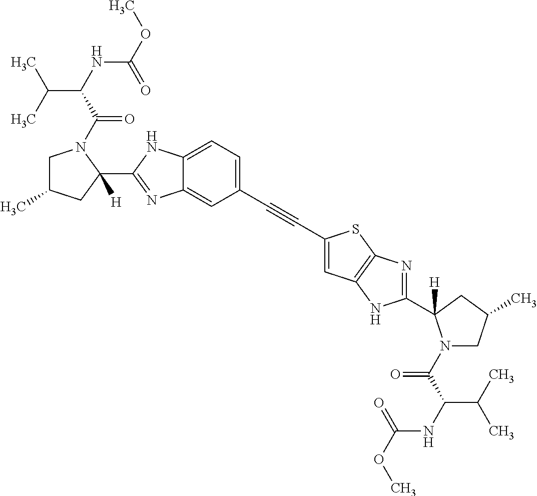 | 0.04034 0.03768993 |
| 3b | 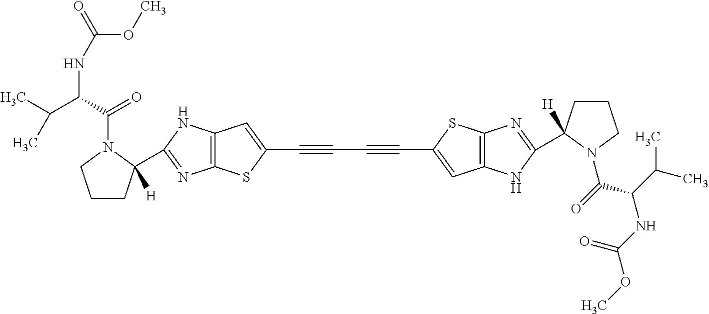 [SMILES: COC(=O)N[C@H](C(=O)N1CCC[C@H]1c1nc2sc(C#CC#Cc3cc4[nH]c([C@@H]5CCCN5C(=O)[C@@H](NC(=O)OC)C(C)C)nc4s3)cc2[nH]1)C(C)C] | 0.2819 |
| 4b | 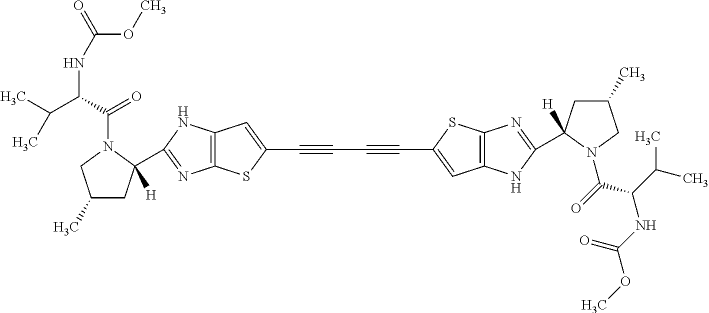 [SMILES: COC(=O)N[C@H](C(=O)N1C[C@@H](C)C[C@H]1c1nc2sc(C#CC#Cc3cc4[nH]c([C@@H]5C[C@H](C)CN5C(=O)[C@@H](NC(=O)OC)C(C)C)nc4s3)cc2[nH]1)C(C)C] | 0.1844 |

Table 4 shows comparative data for exemplary compounds of formula (I) that have a substituent at the 4-position of the pyrrolidine ring (i.e., compounds of the invention where R₄ and R_{4′} are methyl). Data shows IC₅₀ and IC₉₉ values against the sub-genomic replicon 1a and 1b cell lines. According to an aspect of the invention, the compounds of the invention are selected from Table 4 or a pharmaceutically acceptable salt thereof.

TABLE 4

| | | IC₅₀ | IC₉₉ | IC₅₀ | IC₉₉ |
|---|---|---|---|---|---|
| | | (nM) | (nM) | (nM) | (nM) |
| Entry | Structure | (1a) | (1a) | (1b) | (1b) |
| 1c | 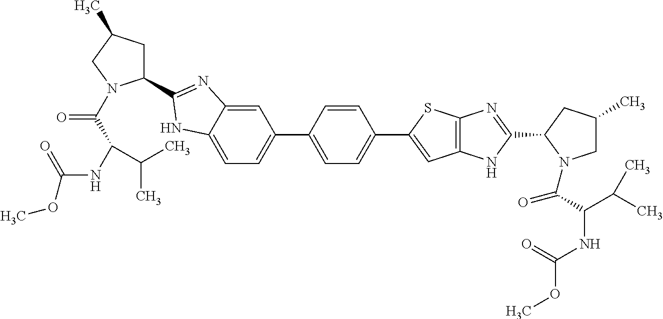 | 1.651 | 11.155 | 0.011 | 0.207 |
| 2c | 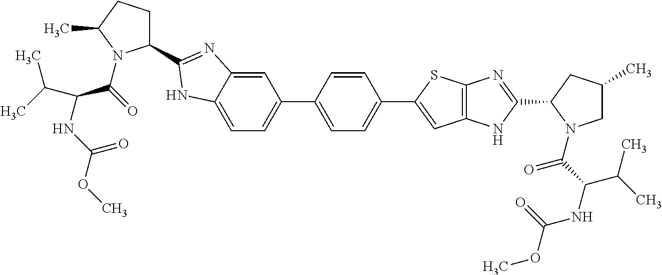 [SMILES: COC(=O)N[C@H](C(=O)N1C[C@@H](C)C[C@H]1c1nc2sc(-c3ccc(-c4ccc5[nH]c([C@@H]6CC[C@H](C)N6C(=O)[C@@H](NC(=O)OC)C(C)C)nc5c4)cc3)cc2[nH]1)C(C)C] | 0.619 | 10.038 | 0.005 | 0.303 |

1. A compound of formula (I): 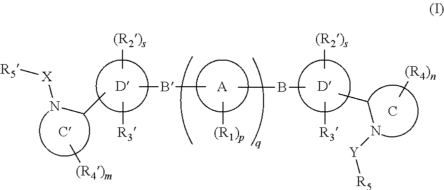
or a pharmaceutically acceptable salt thereof, wherein
each A is independently C₆₋₁₄ aryl, 4-12 membered heterocycle, C₃₋₁₀ cycloalkyl, or 5-12 membered heteroaryl;
B and B′ are each independently absent, C₁₋₆ alkyl, C₂₋₆ alkenyl, or C₂₋₆ alkynyl;
C and C′ are each independently a 4-7 membered heterocycle;
D is a 5,5 membered heterocyclic ring comprising at least one nitrogen atom in the five membered ring adjacent to ring C;
D′ is a 5, 5,6, or a 5,5 membered heterocyclic ring comprising at least one nitrogen atom in the five membered ring adjacent to ring C;
R₁ is halogen, —ORₐ, —NRₐR_{b}, —C(═O)ORₐ, C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, —P(═O)ORₐOR_{b}, C₁₋₆ alkyl which is unsubstituted or substituted one or more times by R¹⁰, C₂₋₆ alkenyl which is unsubstituted or substituted one or more times by R¹⁰, C₂₋₆ alkynyl which is unsubstituted or substituted one or more times by R¹⁰, or any two occurrences of R₁ can be taken together with the atoms to which they are attached to form a 5-7 cycloalkyl which is unsubstituted or substituted one or more times by R¹¹ or a 5-7 membered heterocycle which is unsubstituted or substituted one or more times by R₁₂;
Rₐ—R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl;
Each R₂ and R_{2′} is independently halogen, C₁₋₁₀ alkyl, C₁₋₆ halogenated alkyl, —(CH₂)₁₋₆OH, —ORₐ, —C(═O)ORₐ, —NRₐR_{b}, —NR_{b}C(═O)Rₐ, —C(O)NRₐR_{b}, —S(O)₀₋₃Rₐ, C₆₋₁₂ aryl, 5-12 membered heterocycle, or 5-12 membered heteroaryl;
R₃ and R₃′ are each independently H, C₁₋₆alkyl, —(CH₂)₁₋₆OH, C₂₋₆alkenyl, or C₂₋₆ alkynyl;
R₄ and R₄′ are each independently halogen, —NRₐR_{b}, —C(O)NRₐR_{b}, —(CH₂)₁₋₆OH, C₁₋₆ alkyl, C₁₋₆ halogenated alkyl, hydroxyl, C₆₋₁₄ aryl, or C₁₋₆ alkoxy; wherein two occurrence of R₄ can be taken together with the atoms to which they are attached to form a C₁₋₆ alkenyl which is unsubstituted or substituted one or more times by R¹⁰, a 3-7 cycloalkyl which is unsubstituted or substituted one or more times by R¹¹ or a 4-7 membered heterocycle which is unsubstituted or substituted one or more times by R¹²; wherein two occurrence of R₄′ can be taken together with the atoms to which they are attached to form a C₁₋₆ alkenyl which is unsubstituted or substituted one or more times by R¹⁰, a 3-7 cycloalkyl which is unsubstituted or substituted one or more times by R¹¹ or a 4-7 membered heterocycle which is unsubstituted or substituted one or more times by R¹²;
X and Y are each independently 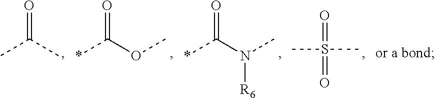
wherein the asterisk (*) indicates the point of attachment to the nitrogen of ring C or C′;
R₅ and R₅′ are each independently H, C₁₋₁₈ alkyl which is unsubstituted or substituted one or more times by R¹⁰, C₂₋₁₂ alkenyl which is unsubstituted or substituted one or more times by R¹⁰, C₂₋₁₂ alkynyl which is unsubstituted or substituted one or more times by R¹⁰, C₆₋₁₄ aryl which is unsubstituted or substituted one or more times by R¹¹, C₇₋₁₆ aralkyl which is unsubstituted or substituted one or more times by R¹¹, 5-12 membered heteroaryl which is unsubstituted or substituted one or more times by R¹¹, 6-18 membered heteroaralkyl which is unsubstituted or substituted one or more times by R¹¹, 3-12 membered heterocycle which is unsubstituted or substituted one or more times by R¹², or 4-18 membered heterocycle-alkyl which is unsubstituted or substituted one or more times by R¹²;
R₆ is H, C₁₋₆ alkyl, or halogenated C₁₋₆ alkyl;
m, and n, are each independently 0, 1, 2, 3 or 4;
p is 0, 1, 2, 3 or 4;
q is 0, 1 or 2;
s is 0, 1, 2, 3 or 4;
R¹⁰ is halogen, —ORₐ, Oxo, NRₐR_{b}, ═NO—Rₑ, —C(═O)ORₐ, C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b};
R¹¹ is halogen, —ORₐ, NRₐR_{b}, —C(═O)ORₐ, C(O)NRₐR_{b}, —C(═O)OH, —C(═O)Rₐ, —C(═NOR_{c})Rₐ, —C(═NR_{c′})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl; and
R¹² is halogen, —ORₐ, Oxo, NRₐR_{b}, ═NO—R_{c}, —C(═O)ORₐ, C(O)NRₐR_{b}, —C(═O)OH, —C(═O)Rₐ, —C(═NOR_{c})Rₐ, —C(═NR_{c})NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, —NR_{b}SO₂NRₐR_{b}, or —P(═O)ORₐOR_{b}, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

2. The compound according to claim 1, wherein said compound is of formula (IA): 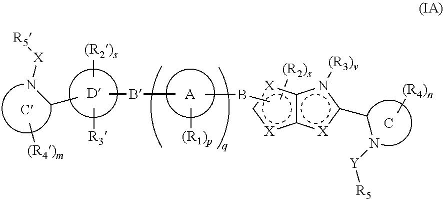
wherein:
D′ is selected from the group consisting of: 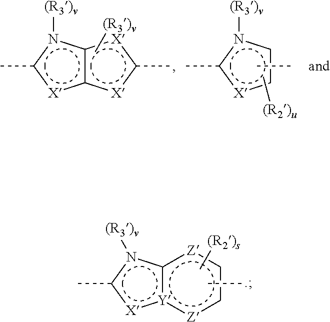
each X and X′ is independently —N—, —S—, or —CH—;
each Z′ is independently —N— or —CH—;
u is 0 or 1; and
each v is independently 0 or 1.

3. The compound according to claim 1, wherein said compound is of formula (II): 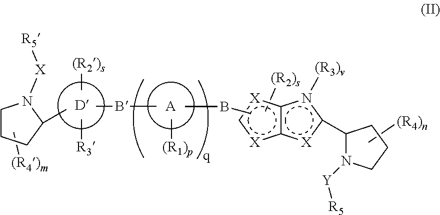
or a pharmaceutically acceptable salt thereof.

4. The compound according to claim 1, wherein said compound is of formula (IIIA): 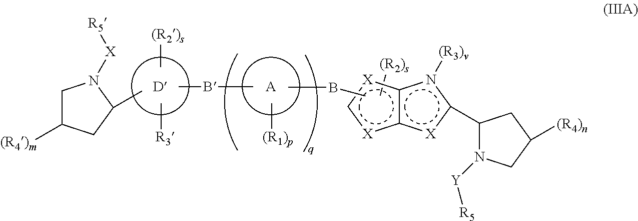
or a pharmaceutically acceptable salt thereof wherein
m and n combined are 1, 2, 3, or 4.

5. The compound according to claim 1, wherein said compound is of formula (IIIB): 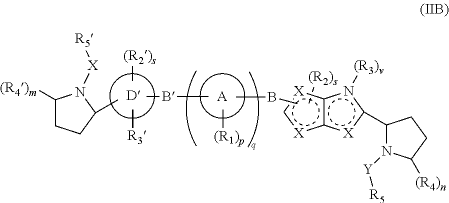
or a pharmaceutically acceptable salt thereof wherein
m and n combined are 1, 2, 3, or 4.

6. The compound according to any one of claims 1 to 5, wherein
each A is independently cyclopropyl, cyclohexyl, pyrrolidinyl, pyrazolidinyl, imidazolidinyl, piperazinyl, piperadinyl, phenyl, naphthalenyl, thienyl, furanyl, pyrrolyl, pyrazolyl, imidazolyl, thiazolyl, thiadiazolyl, oxazolyl, oxadiazolyl, pyridyl, pyrimidyl, pyrazinyl, pyridazinyl, indolyl, indazolyl, benzimidazolyl, benzoxazolyl, benzodioxolyl, benzothiazolyl, benzothiadiazolyl, dihydrobenzodioxine, thienofuranyl, thienothienyl, thienopyrrolyl, quinolinyl, quinoxalinyl, quinazolinyl, cinnolinyl, or triazolyl; and wherein each A is independently substituted with (R₁)ₚ.

7.-12. (canceled)

13. The compound according to claim 1, wherein B and B′ are independently absent, C₁₋₆ alkyl or C₂₋₆ alkynyl.

14.-16. (canceled)

17. The compound according to claim 1, wherein 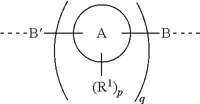
is selected from the group consisting of:

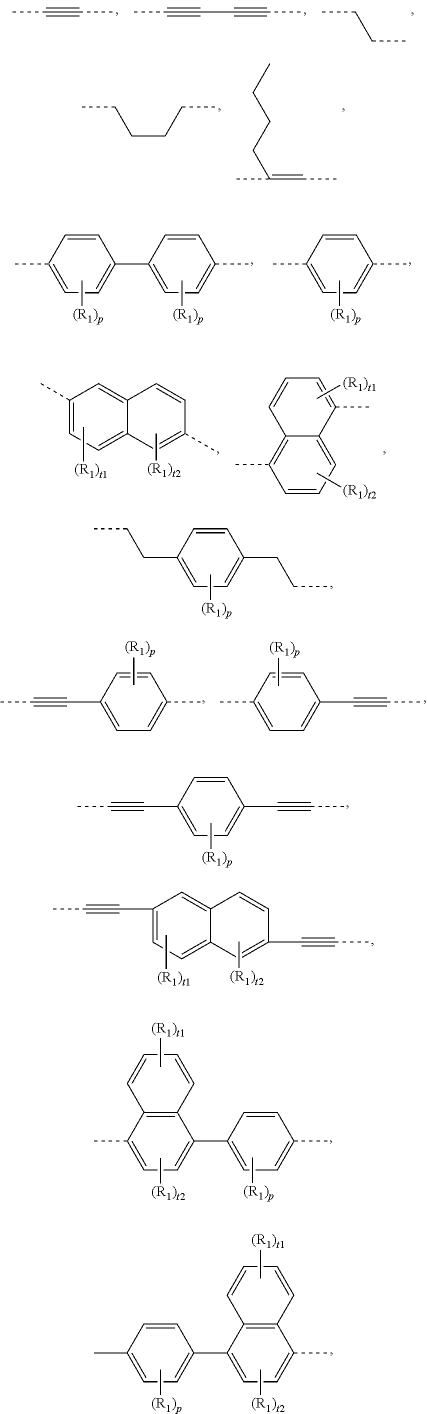

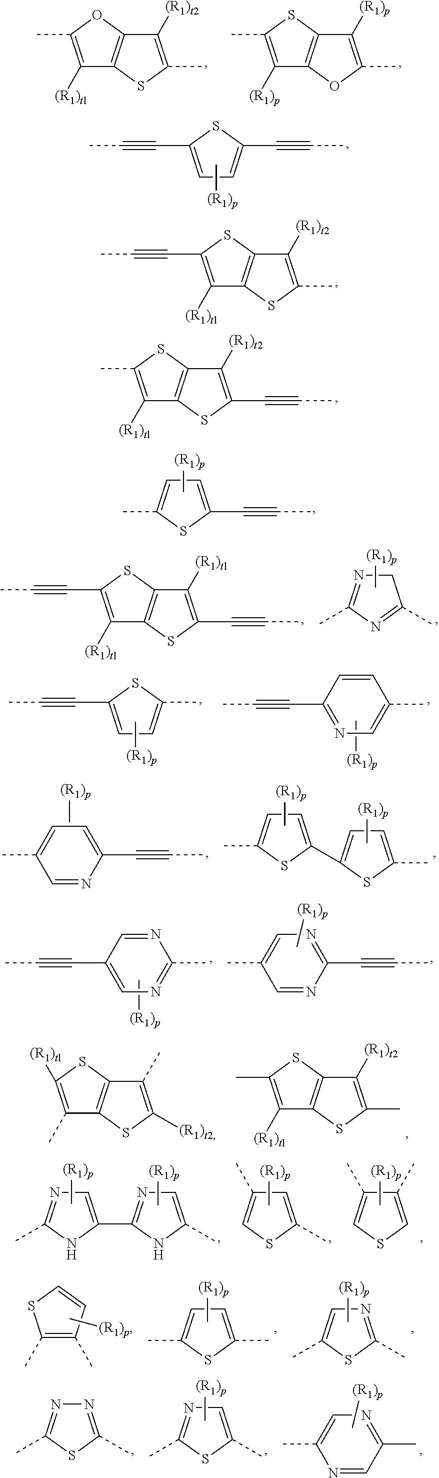

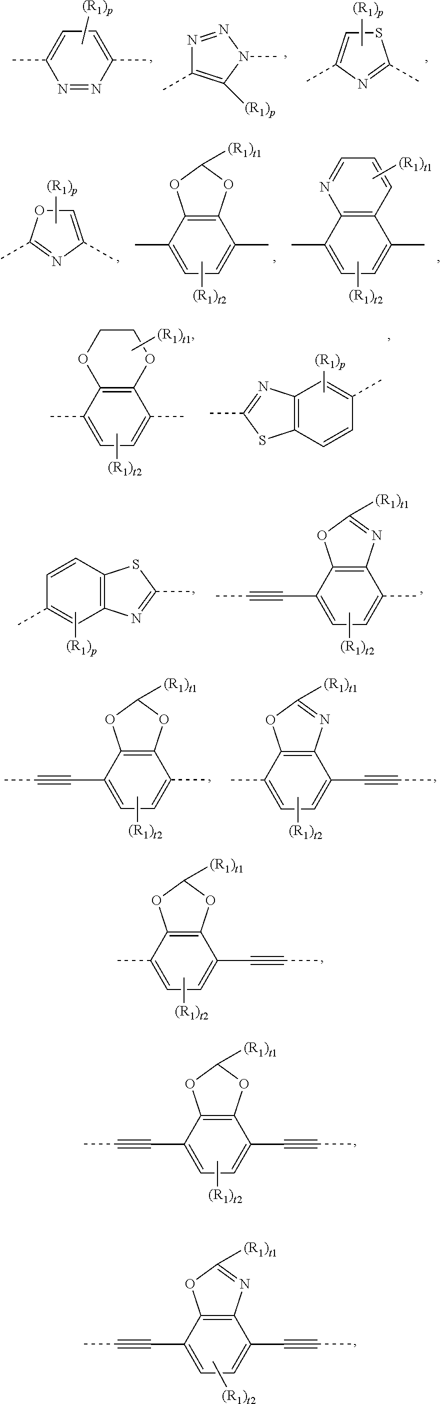

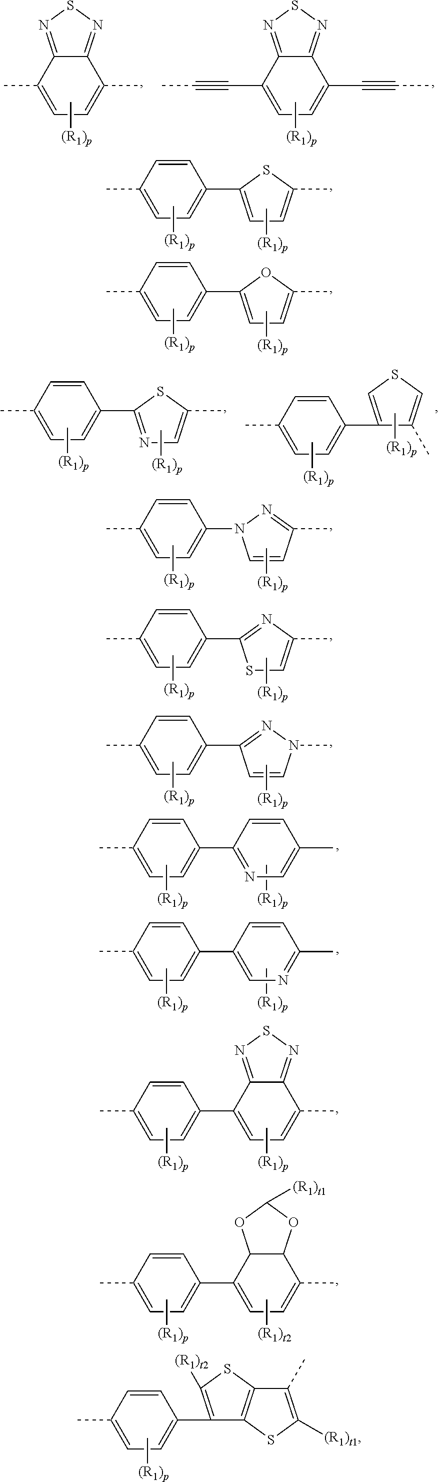

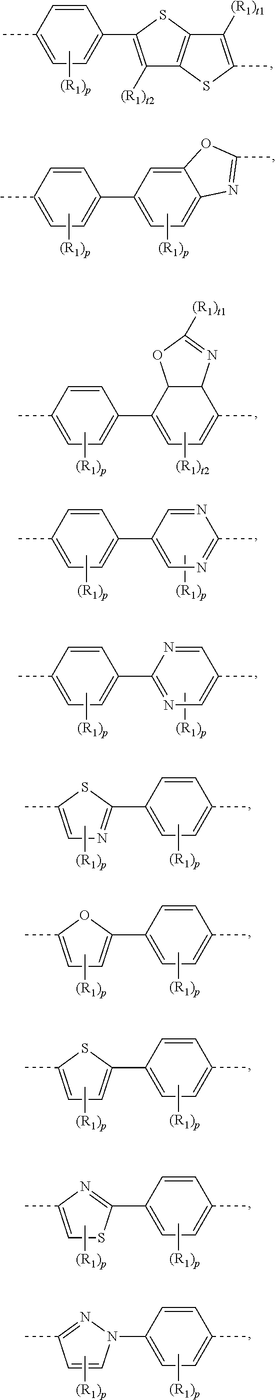

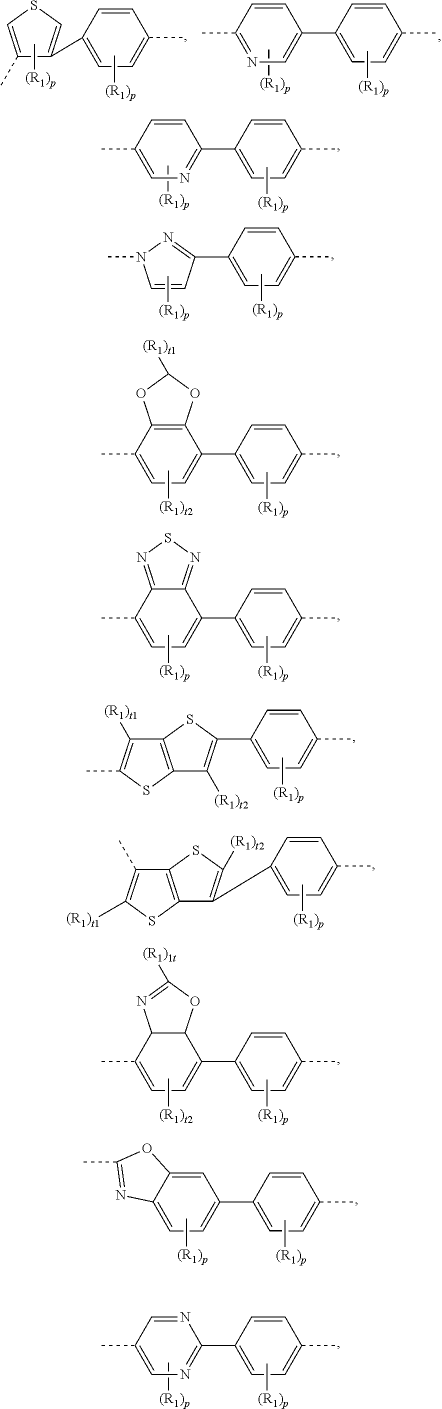

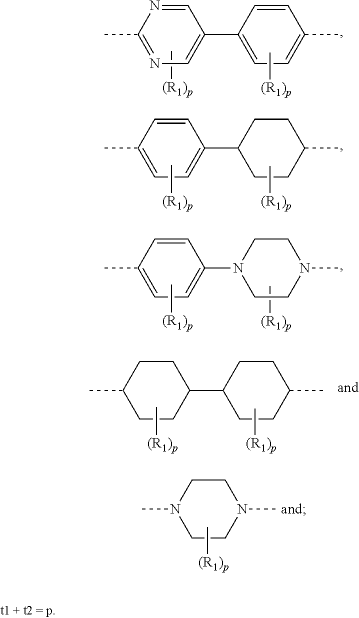

18.-23. (canceled)

24. The compound according to claim 1, wherein R₁ is halogen, C₁₋₄ alkyl which is unsubstituted or substituted one or more times by R¹⁰, —C(═O)ORₐ, —C(O)NRₐR_{b}, hydroxyl, cyano, or C₁₋₃ alkoxy.

25. The compound according to claim 24, wherein R₁ is chloro, fluoro, bromo, methyl, ethyl, propyl, butyl, —CH₂OH, difluoromethyl, trifluoromethyl, —C(═O)ORₐ, hydroxyl, cyano, or methoxy.

26. The compound according to claim 1, wherein R₂′ is fluoro, methyl, trifluoromethyl, iodo, CH₂OH, or NHC(O)CH₃.

27. The compound according to claim 26, wherein s is 0.

28. The compound according to claim 1, wherein each R₂ is independently fluoro or methyl.

29. The compound according to claim 28, wherein s is 0.

30. The compound according to claim 1, wherein R₃ and R₃′ are H or methyl.

31. The compound according to claim 1, wherein R₄ and R₄′ are each independently halogen, methyl, ethyl, isopropyl, di-fluoromethyl, di-fluoroethyl, trifluoromethyl, tri-fluoroethyl, —CH₂OH, —NRₐN_{b}, t-butoxy-, or hydroxyl; or two R₄ groups together with the atoms to which they are attached form fused cyclopropyl, spiro cyclopropyl or 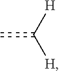
two R₄′ groups together with the atoms to which they are attached form fused cyclopropyl, spiro cyclopropyl or

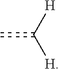

32.-33. (canceled)

34. The compound according to claim 1, wherein m and n are independently 1 or 2.

35. (canceled)

36. The compound according to claim 1, wherein X and Y are

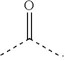

37. The compound according to claim 1, wherein R₅ and R₅′ are each independently, C₁₋₈ alkyl which is unsubstituted or substituted one or more times by R¹⁰, C₂₋₈ alkenyl which is unsubstituted or substituted one or more times by R¹⁰, C₂₋₈ alkynyl which is unsubstituted or substituted one or more times by R¹⁰, phenyl which is unsubstituted or substituted one or more times by R¹¹, C₇₋₈ aralkyl which is unsubstituted or substituted one or more times by R¹¹, 5-6 membered heteroaryl which is unsubstituted or substituted one or more times by R¹¹, 6-8 membered heteroaralkyl which is unsubstituted or substituted one or more times by R¹¹, 3-6 membered heterocycle which is unsubstituted or substituted one or more times by R¹², or 4-8 membered heterocycle-alkyl which is unsubstituted or substituted one or more times by R¹².

38.-42. (canceled)

43. The compound according to claim 1, wherein R¹⁰ is halogen, —ORₐ, oxo, —NRₐR_{b}, ═NO—R_{c}, —C(═O)ORₐ, —C(O)NRₐR_{b}, —C(═O)OH, —C(═O)Rₐ, —C(═NOR_{c})Rₐ, —C(═NR_{c′})NRₐR_{b}, NR_{d}C(═O)NRₐR_{b}, —NR_{b}C(═O)Rₐ, NR_{d}C(═NR_{c})NRₐR_{b}, —NR_{b}C(═O)ORₐ, —OC(═O)NRₐR_{b}, —OC(═O)Rₐ, —OC(═O)ORₐ, hydroxyl, nitro, azido, cyano, —S(O)₀₋₃Rₐ, —SO₂NRₐR_{b}, —NR_{b}SO₂Rₐ, or —NR_{b}SO₂NRₐR_{b}, wherein Rₐ—R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl.

44.-45. (canceled)

46. The compound according to claim 1, wherein Rₐ—R_{d} are each independently H, C₁₋₆ alkyl, C₂₋₆ alkenyl, C₂₋₆ alkynyl, phenyl, C₇₋₈ aralkyl, 5-6 membered heteroaryl, 6-8 membered heteroaralkyl, 5-6 membered heterocycle, or 6-8 membered heterocycle-alkyl.

47.-48. (canceled)

49. The compound according to claim 1, wherein said compound is of formula (IIIC): 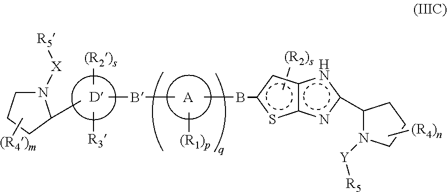
wherein D′ is selected from the group consisting of:

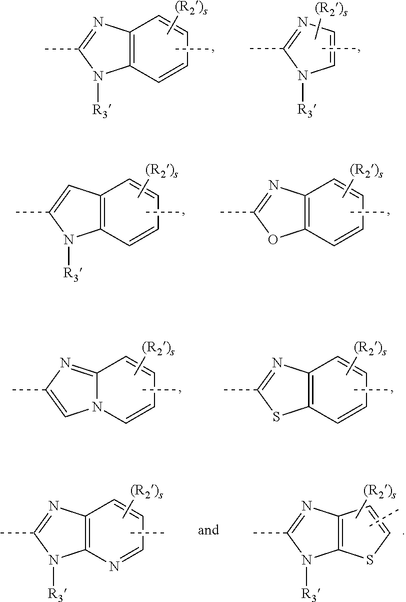

50. The compound according to claim 1, wherein said compound is of formula (IV): 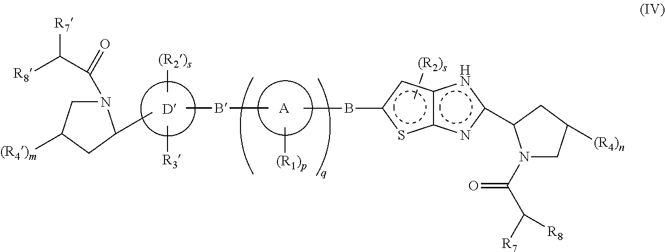
or a pharmaceutically acceptable salt thereof wherein
R₇ and R₇′ are each independently C₁₋₈ alkyl which is unsubstituted or substituted one or more times by R¹⁰, C₂₋₈ alkenyl which is unsubstituted or substituted one or more times by R¹⁰, C₂₋₈ alkynyl which is unsubstituted or substituted one or more times by R¹⁰, phenyl which is unsubstituted or substituted one or more times by R¹¹, benzyl which is unsubstituted or substituted one or more times by R¹¹, 5-6 membered heteroaryl which is unsubstituted or substituted one or more times by R¹¹, 6-7 membered heteroaralkyl which is unsubstituted or substituted one or more times by R¹¹, 3-6 membered heterocycle which is unsubstituted or substituted one or more times by R¹², or 4-7 membered heterocycle-alkyl which is unsubstituted or substituted one or more times by R¹²;
R₈ and R₈′ are each independently —NRₐR_{b}, —NR_{d}C(═O)NRₐR_{b}, —NR_{b}C(═O)Rₐ, —NR_{d}C(═NRₐ)NRₐR_{b}, —NR_{b}C(═O)ORₐ, —NR_{b}SO₂Rₐ, or —NR_{b}SO₂NRₐR_{b}, wherein Rₐ—R_{d} are each independently H, C₁₋₁₂alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl; and
m and n combined are 0, 1, 2, 3 or 4.

51.-56. (canceled)

57. The compound according to claim 1, wherein said compound is of formula (V): 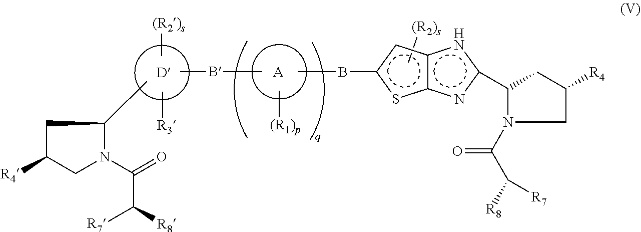
or a pharmaceutically acceptable salt thereof.

58. The compound according to claim 2, wherein said compound is of formula (IV): 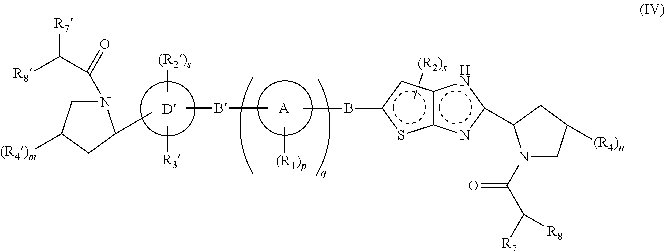
or a pharmaceutically acceptable salt thereof, wherein 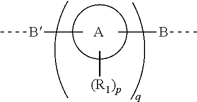
is selected from the group consisting of: 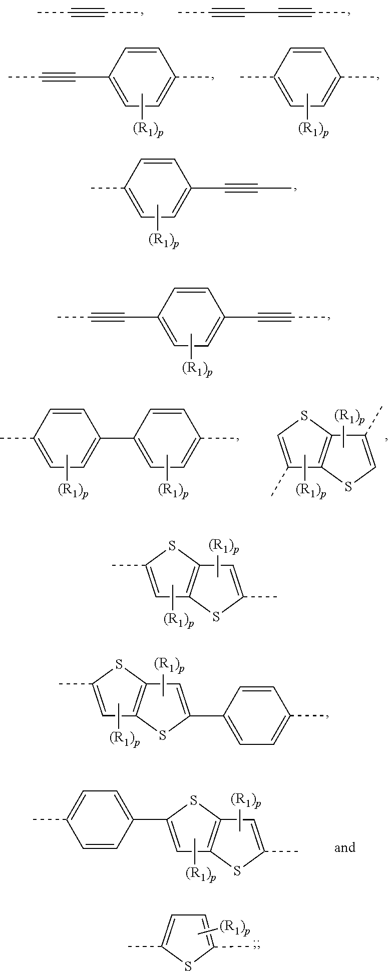
R₇ and R₇′ are each independently C₁₋₈ alkyl which is unsubstituted or substituted one or more times by R¹⁰, C₂₋₈ alkenyl which is unsubstituted or substituted one or more times by R¹⁰, C₂₋₈ alkynyl which is unsubstituted or substituted one or more times by R¹⁰, phenyl which is unsubstituted or substituted one or more times by R¹¹, benzyl which is unsubstituted or substituted one or more times by R¹¹, 5-6 membered heteroaryl which is unsubstituted or substituted one or more times by R¹¹, 6-7 membered heteroaralkyl which is unsubstituted or substituted one or more times by R¹¹, 3-6 membered heterocycle which is unsubstituted or substituted one or more times by R¹², or 4-7 membered heterocycle-alkyl which is unsubstituted or substituted one or more times by R¹²;
R₈ and R₈′ are each independently —NRₐR_{b}, —NR_{d}C(═O)NRₐR_{b}, —NR_{b}C(═O)Rₐ, —NR_{d}C(═NRₐ)NRₐR_{b}, —NR_{b}C(═O)ORₐ, —NR_{b}SO₂Rₐ, or —NR_{b}SO₂NRₐR_{b}, wherein Rₐ—R_{d} are each independently H, C₁₋₁₂ alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl; and
m and n combined are 0, 1, 2, 3 or 4.

59. The compound of claim 58, wherein said compound is of formula (V): 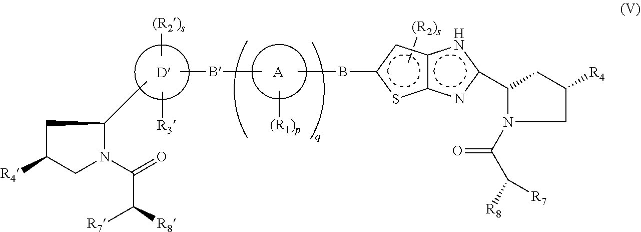
or a pharmaceutically acceptable salt thereof.

60.-67. (canceled)

68. The compound according to claim 2, wherein said compound is of formula (VI): 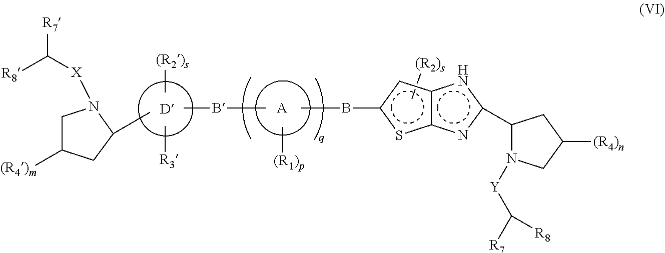
or a pharmaceutically acceptable salt thereof; and wherein R₇ and R₇′ are each independently C₁₋₈ alkyl which is unsubstituted or substituted one or more times by R¹⁰, C₂₋₈ alkenyl which is unsubstituted or substituted one or more times by R¹⁰, C₂₋₈ alkynyl which is unsubstituted or substituted one or more times by R¹⁰, phenyl which is unsubstituted or substituted one or more times by R¹¹, benzyl which is unsubstituted or substituted one or more times by R¹¹, 5-6 membered heteroaryl which is unsubstituted or substituted one or more times by R¹¹, 6-7 membered heteroaralkyl which is unsubstituted or substituted one or more times by R¹¹, 3-6 membered heterocycle which is unsubstituted or substituted one or more times by R¹², or 4-7 membered heterocycle-alkyl which is unsubstituted or substituted one or more times by R¹²;
R₈ and R₈′ are each independently —NRₐR_{b}, —NR_{d}C(═O)NRₐR_{b}, —NR_{b}C(═O)Rₐ, —NR_{d}C(═NR_{c})NRₐR_{b}, —NR_{b}C(═O)ORₐ, —NR_{b}SO₂Rₐ, or —NR_{b}SO₂NRₐR_{b}, wherein Rₐ—R_{d} are each independently H, C₁₋₁₂alkyl, C₂₋₁₂ alkenyl, C₂₋₁₂ alkynyl, C₆₋₁₂ aryl, C₇₋₁₆ aralkyl, 5-12 membered heteroaryl, 6-18 membered heteroaralkyl, 3-12 membered heterocycle, or 4-18 membered heterocycle-alkyl; and
m and n combined are 0, 1, 2, 3 or 4.

69. The compound according to claim 68, wherein said compound is of formula (VII): 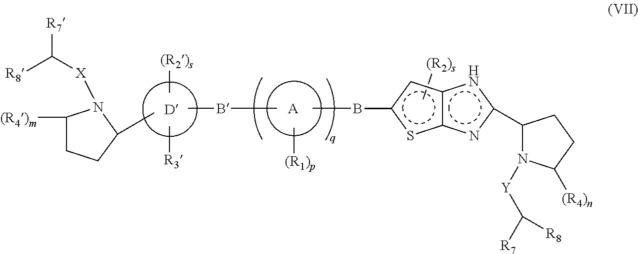
or a pharmaceutically acceptable salt thereof.

70.-92. (canceled)

93. The compound according to claim 58, wherein said compound is of formula (VIII): 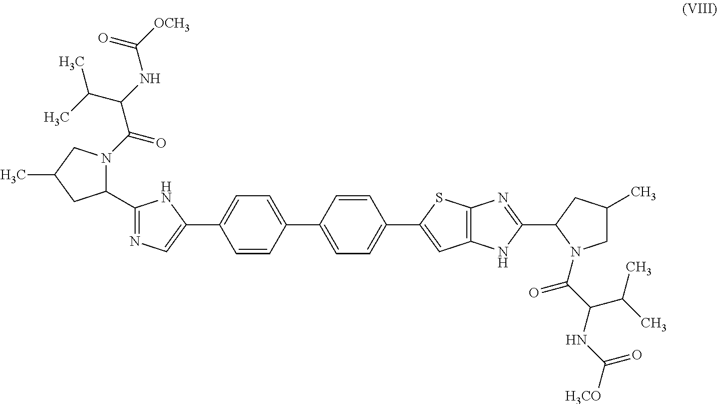 [SMILES: COC(=O)NC(C(=O)N1CC(C)CC1c1ncc(-c2ccc(-c3ccc(-c4cc5[nH]c(C6CC(C)CN6C(=O)C(NC(=O)OC)C(C)C)nc5s4)cc3)cc2)[nH]1)C(C)C]
or a pharmaceutically acceptable salt thereof.

94. The compound according to claim 58, wherein said compound is of formula (IX): 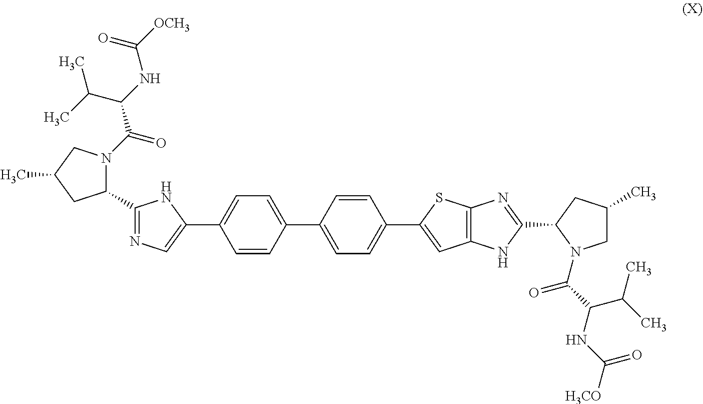
or a pharmaceutically acceptable salt thereof.

95. The compound selected from Tables 1A, 1B, 3, or 4 or a pharmaceutically acceptable salt thereof.

96. (canceled)

97. A pharmaceutical composition comprising at least one compound according to claim 1 and at least one pharmaceutically acceptable carrier or excipient.

98. A method of treating or preventing infection by a HCV virus, comprising contacting a biological sample or administering to a patient in need thereof a compound of claim 1 in an amount effective to treat or prevent the infection.

99.-100. (canceled)
